(19) United States
(12) Patent Application Publication
(10) Pub. No.: US 2002/0049190 A1
(43) Pub. Date: Apr. 25, 2002

(54) Pharmaceutical compositions comprising metal complexes

(76) Inventors: Gary J. Bridger, Bellingham, WA (US); Beth R. Cameron, Langley (CA); Simon P. Fricker, Langley (CA); Michael J. Abrams, Custer, WA (US); Renato Skerlj, Blaine, WA (US); Ian Baird, Vancouver (CA)

Correspondence Address: Thomas D Mays Ph D JD, Morrison & Foerster LLP, 2000 Pennsylvania Avenue NW, Suite 5500, Washington, DC, 20006-1888, US

(21) Appl. No.: 09/527,450

(22) Filed: Mar. 17, 2000

Related U.S. Application Data
(60) Provisional application No. 60/125,166, filed on Mar. 19, 1999.

(51) Int. Cl. C07F 15/00; A61K 31/555; A61K 31/295; A61P 35/00

(52) U.S. Cl. 514/188; 514/492; 544/64; 546/2; 546/10; 540/145; 556/136; 556/137; 548/403; 544/181; 546/6; 540/465; 548/101

(57) ABSTRACT

A compound of the formula

[Mₐ(X_{b}L)_{c}Y_{d}Zₑ]^{nt±} Formula I

wherein:

M is a metal ion or a mixture of metal ions;

X is a cation or a mixture of cations;

L is a ligand, or mixture of ligands each containing at least two different donor atoms selected from the elements of Group IV, Group V or Group VI of the Periodic Table;

Y is a ligand or a mixture of the same or different ligands each containing at least one donor atom or more than one donor atom selected from the elements of Group IV, Group V or Group VI of the Periodic Table; and

Z is a halide or pseudohalide ion or a mixture of halide ions and pseudohalide ions; and

wherein: a=1-3; b=0-12; c=0-18; d=0-18; e=0-18; and n=0-10; provided that at least one of c, d and e is 1 or more;

wherein c is 0: b is also 0;

wherein a is 1: c, d and e are not greater than 9; and

wherein a is 2: c, d and e are not greater than 12.

### TECHNICAL FIELD

This invention relates to new pharmaceutical compositions and to pharmaceutical compositions having activity against diseases caused by, or related to, overproduction of localised high concentrations of reaction oxygen species, including nitric oxide, in the body.

### BACKGROUND

Nitric oxide (NO) plays a varied and vital role in the body of a human or other mammals. For example, NO plays a vital role in the control of blood pressure: it acts as a neurotransmitter; it plays a role in inhibition of platelet aggregation (important in thrombosis or blockages of the blood vessels) and in cytostasis (important in fighting of tumours). Overproduction of NO however, has been implicated in a number of disease states, including vascular/pressor diseases such as septic shock, post-ischaemic cerebral damage, migraine and dialysis induced renal hypotension: immunopathologic diseases such as hepatic damage in inflammation and sepsis allograft rejection, graft versus host diseases, diabetes and wound healing: neurodegenerative diseases such as cerebral ischaemia, trauma, chronic epilepsy, Alzheimer's disease, Huntington's disease, and AIDS dementia complex; and side effects of treatment such as restenosis following angioplastic treatment and secondary hypotension following cytokine therapy.

Pharmacological modulation of nitric oxide or other reactive oxygen species in any of these disease states should prove extremely beneficial.

One above-mentioned disease relating to overproduction of NO is septic shock. This is precipitated by local septicaemnia or endotoxaemia, (high local levels of bacterial endotoxins). The result is activation of macrophages, lymphocytes, endothelial cells and other cell types capable of producing NO further mediated by cytokine production by these cells. The activated macrophages produce excess NO which causes vasodilation of the blood vessels, and results in local vascular damage and vascular collapse. This destruction of vascular integrity may be so great that it leads to the collapse of haemodynanic homeostasis, the end result being death.

Current ideas for pharmacological modulation of nitric oxide in such diseases are based on dealing with the mediators of septic shock such as cytokines, endotoxins and platelet activating factor (PAF). The approaches include use of antibodies to cytokines such as tumour necrosis factor (TNF) receptor antagonists such as interleukin I (IL-1) antibodies to lipopolysaccharide (the endotoxins produced by Gram negative bacteria) and PAF antagonists. All such approaches while challenging a factor mediating septic shock do not attempt to deal with the aetiology or cause of the disease. Recent advances in understanding of NO have lead to the proposal that inhibitors of the NO synthase enzyme such as N^{G}-monomethy-L-arginine (L-NMMA) may be useful in the treatment of septic shock and other NO overproduction related to diseases since they inhibit NO production. While these inhibitors have shown some utility in animal models and preliminary clinical studies they have the disadvantage of undesirably inhibiting total NO synthesis in the body.

An aim of the present invention is to provide new compositions which are able to modulate levels of NO and other reactive oxygen species in the body. Examples of other reactive species include superoxide, hydroxyl radical, peroxide, peroxynitrite, and other oxides of nitrogen including protein adducts. The compositions of metal complexes described herein are able to carry out the important role of reducing levels of these harmful species by scavenging.

### SUMMARY OF THE INVENTION

Some metal complexes are known in pharmaceutical compositions for the treatment of diseases of the body of a human or other mammal. For example certain complexes of platinum and ruthenium have been used or indicated in the treatment of cancer. Metal complexes have not however been previously indicated in the treatment of disease relating to the overproduction of reactive oxygen species (including the overproduction of NO).

This invention provides for the use of a neutral anionic or cationic metal complex having at least one site for coordination with NO of Formula I

[Mₐ(XbL)_{c}Y_{d}Zₑ]^{nt±}Formula I

in the manufacture of a medicament for the attenuation of NO levels and other reactive oxygen species when implicated in disease.

where:

M is a metal ion or a mixture of metal ions:

X is a cation or a mixture of cations:

L is a ligand, or mixture of ligands each containing at least two different donor atoms selected from the elements of Group IV, Group V or Group VI of the Periodic Table;

Y is a ligand or a mixture of the same or different ligands each containing at least one donor atom or more than one donor atom which donor atom is selected from the elements of Group IV, Group V or Group VI of the Periodic Table:

And

Z is a halide or pseudohalide ion or a mixture of halide ions and pseudohalide ions:

a=1-3; b=0-12; c=0-18; d=0-18; e=0-18; and n=O-10; provided that at least one of c, d and e is 1 or more.

And where c is 0: b is also 0;

And where a is 1: c, d and e are not greater than 9;

And where a is 2: c, d and e are not greater than 12.

By “complex” in this specification is meant a neutral complex or anionic or cationic species.

The term “Group” which is used herein is to be understood as a vertical column of the periodic table in which elements of each Group have similar physical and chemical properties. The definition of the Periodic Table is that credited to Mendeleev; Chamber Dictionary of Science and Technology, 1974 Published by W & R Chambers Ltd. The nomenclature of the compounds as disclosed herein are based upon common usage. The names of the compounds according to nomenclature of the American Chemical Abstracts Service (American Chemical Society) are also provided in Table 5.

This invention may also be stated as providing a method of attenuation of reactive oxygen species when implicated in diseases of the human body or the bodies of other mammals. Thus the invention comprises administering a pharmaceutical composition containing a neutral, anionic or cationic metal complex of Formula I.

This invention may also provide for the use of a neutral anionic or cationic metal complex of formula I in the manufacture of a medicament for the treatment of diseases in which reactive oxygen species are overproduced.

This invention may also be stated as providing a method of attenuation of nitric oxide when implicated in diseases of the human body or bodies of other mammals. Thus the invention comprises administering a pharmaceutical composition containing a neutral, anionic or cationic metal complex of Formula I.

This invention may also be stated as providing a method of treatment of diseases of a body of a human or other mammals resultant of overproduction of NO in the body comprising administering a pharmaceutical composition containing a neutral anionic or cationic metal complex of formula I.

Where the formula I represents an anionic species a cation will also be present. Where formula I represent a cationic species an anion will also be present. The metal complexes may be hydrated.

Preferably M is a first, second or third row transition metal ion. For example M may be an Rh, Ru, Os, Mn, Co, Cr or Re ion, and is preferably an Rh, Ru or Os ion.

Suitably M is in an oxidation state III. We have found surprisingly that when the metal ion for example ruthenium is in oxidation state III, the rate at which it binds with NO is significantly faster than when it is in oxidation state II.

X may be any cation, such as mono-, di- or tri-valent cation. Suitable cations may be H⁺, K⁺, Na⁺, NH₄⁺ or Ca²⁺. Conveniently X may be H⁺, K⁺, or Na⁺.

Preferably L is a polyaminocarboxylate ligand described herein by the general formulae A and B:

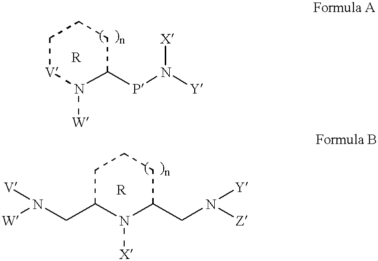

Where:

V′, W′, X′, Y′ and Z′ are independently selected selected from H, phenyl, heteroaryl, C₁₋₆alkyl, C₁₋₆alkylhydroxy, C₁₋₆alkylthiol, C₁₋₆alkylaryl, C₁₋₆alkylheteroaryl, C₁₋₆alkylheterocyclyl and derivatives thereof. Preferred alkylheterocyclic groups are pyridinylmethylene, pyrazinylmethylene, pyrimidinylmethylene. The aromatic and heteroaromatic groups may be suitably substituted in single or multiple positions with halide, C₁₋₆alkyl, C₁₋₆alkoxy, C₁₋₆alkoxyaryl or benzyloxy, hydroxy, C₁₋₆hydroxyalkyl, thiol, carboxylic acid, carboxyalkylC₁₋₆, carboxamide, carboxamidoalkylC₁₋₆, anilide.

P′=CH₂, (CH₂)₂, CHOHCH₂, CH(OC₁₋₆alkyl)CH₂

V′, W′, X′, Y′ and Z′ may also be methylenecarboxylic acid, methylenecarboxyC₁₋₆alkyl, methylenecarboxamideC₁₋₆alkyl or heterocyclyl, methylenecarboxanilide, methylenecarboxamido derivatives of an aminoacid or peptide, methylenehydroxamic acid, methylene phosphonic acid, C₁₋₆alkylthiol.

In the above formulae, the ligands may be optionally fused with a heterocyclic ring R (n=0 or 1). Prefered heterocyclic groups are pyridine, pyrimidine, pyrazine, imidazole, thiazole, oxazole.

More preferably L is a ligand such as ethylenediamine-N,N′-diacetic acid (edda), ethylenediaminetetraacetic acid (edta), nitrilotriacetic acid (nta), dipicolinic acid (dipic), picolinic acid (pic), diethylenetri-aminepentaacetic acid (dtpa), thiobis(ethylenenitrilo)tetraacetic acid (tedta), dithioethanebis(ethylenenitrilo)tetraacetic acid (dtedta), and N-(2-hydroxethyl) ethylenediamine-triacetic acid (hedtra).

Preferably Y is a ligand containing nitrogen, oxygen, sulphur, carbon or phosphorus donor groups. Suitable nitrogen donor groups may be for example ammine, amine, nitrile and nitride or derivations thereof. Suitable oxygen donor groups may be for example carboxylic acid, ester or derivations thereof, water, oxide, sulphoxide, hydroxide, acetate, lactate, propionate, oxalate and maltolate. Suitable sulphur donor groups may be for example sulphoxide, dialkysulphide, dithiocarbamate or dithiophosphate. Suitable carbon donor groups may be for example carbon monoxide or isocyanide. Suitable phosphorus donor groups may be for example trialkylphosphine.

Z may be any halide and is preferably chloride, bromide or iodide. Most conveniently, Z is chloride.

Examples of metal complexes for use according to the present invention include optionally hydrated ruthenium complexes of Formula II

[Ru(H₀₋₆L^{II})₁₋₃Y₀₋₂Cl₀₋₄]^{(0-4)±} Formula II

where L^{II} is a polyaminocarboxylate ligand as already described herein by the general formulae A and B, more preferably a polydentate aminocarboxylate ligand such as, for example edta, nta, dipic, pic, edda, tropolone, dtpa, hedtra, tedta or dtedta or diamide of edta or dtpa (or an amide or ester derivative thereof) or a mixture of any of these and Y is as defined above and may for example be selected from: acetylacetone (acac) a β-diketonate; water; dimethylsulphoxide (dmso); carboxylate; bidentate carboxylate; catechol; kojiic acid; maltol; hydroxide; tropolone; malonic acid; oxalic acid; 2.3-dihydroxynaphthalene; squaric acid; acetate; a sulphate and a glycolate.

The skilled artisan will be able to substitute other known ligands at Y and which will fall within the scope of the inventions.

Preparative methods of tedta, dtedta and diamide of edta and dtpa are described in the following references respectively:

P Tse & J E Powell, Inorg Chem, (1985), 24, 2727

G Schwartzenbach, H Senner, G Anderegg, Helv Chim Acta 1957, 40, 1886

M S Konings, W C Dow, D B Love, K N Raymond, S C Quay and S M Rocklage, Inorg Chem (1990), 29, 1488-1491

P N Turowski, S J Rodgers, R C Scarrow and K N Raymond, Inorg Chem (1988), 27,474-481.

Where the complex of Formula II is an anion, a cation will be required. For example the complexes of Formula II are present in

K[Ru(Hedta)Cl]2H₂O

[Ru(H₂edta)(acac)]

K[Ru(hedtra)Cl]H₂O

K[Ru(dipic)₂]H₂O

(H₂pic)[RuCl₂(pic)₂](Hpic)H₂O

K[Ru(H₂edta)Cl₂]H₂O

K[Ru(Hnta)₂]½H₂O

K[Ru(H₂dtpa)Cl]H₂O

[Ru(Hhedtra)acac]H₂O

[Ru(Hhedtra)trop]

[Ru(H₃dtpa)Cl]

Complexes of formula II have not to the best of our knowledge been previously indicated in any pharmaceutical composition. Therefore the present invention also provides a pharmaceutical composition containing an optionally hydrated ruthenium complex of Formula II.

Further examples of metal complexes for use according to the present invention include optionally hydrated complexes of Formula III

[M₁₋₃Y₁₋₁₈Cl₀₋₁₈]^{(0-6)±} Formula III

where Y is a sulphur donor ligand. For example, such complex is present in

[Ru(mtc)₃] (mtc=4-morpolinecarbodithoic acid)

Ru(S₂CNCH₂CH₂NMeCH₂CH₂)₃½H₂O

Complexes of Formula III in which Y is a sulphur donor ligand have not to the best of our knowledge been previously indicated in any pharmaceutical composition. Therefore, the present invention also provides a pharmaceutical composition containing an optionally hydrated complex of Formula III when Y is a sulphur donor ligand.

Yet further examples of metal complexes for use according to the present invention include optionally hydrated complexes of ruthenium of Formula III

[M^{III}₁₋₃Y^{III}₁₋₁₈Cl₀₋₁₈]^{(0-6)±} Formula III

where M^{III} is ruthenium and Y^{III} is an oxygen-donor ligand such as acetate, lactate, water, oxide, propionate (COEt), oxalate (ox), or maltolate (maltol) or a combination of these. For example complexes of Formula III are present in

[Ru₃O(OAc)₆](OAc)

[Ru₃O(lac)₆](lac)

[Ru₂(OAc)₄]NO₃

[Ru₂(OCOEt)₄]NO₃

K₃[Ru(ox)₃]

[Ru₂(OAc)₄]Cl

[Ru(maltol)₃]

Some complexes of Formula III have not to the best of our knowledge been previously indicated in any pharmaceutical composition. Therefore the present invention also provides a pharmaceutical composition containing an optionally hydrated complex of ruthenium of Formula III wherein M^{III} is ruthenium and Y^{III} is an oxygen-donor ligand selected from the group acetate, lactate, oxide, propionate and maltolate.

Further examples of metal complexes for use according to the present invention include optionally hydrated complexes of ruthenium of Formula IV

[RuY^{IV}₉Cl₁₋₉]^{(0-4)±} Formula IV

where Y^{IV} is a nitrogen-donor ligand such as: ammine; ethylenediamine (en); pyridine (py); 1,10-phenanthroline (phen): 2,2-bipyridine (bipy) or 1,4,8,11-tetraazacyclotetradecane (cyclam); 1,4,7-triazacyclononane; 1,4,7-triazacyclononane tris acetic acid; 2,3,7,8,12,13,17,18-octaethylporphyrin (oep); or a combination of these. For example complexes of Formula IV are present in

[Ru(H₃N)₅Cl]Cl₂

[Ru(en)₃]I₃

trans-[RuCl₂(py)₄]

K[Ru(phen)Cl₄]

[Ru(cyclam)Cl₂]Cl

K[Ru(bipy)Cl₄]

[Ru(NH₃)₆]Cl₃

[Ru(NH₃)₄Cl₂]Cl

Ru(oep)Ph

Some complexes of Formula IV have not to the best of our knowledge been previously indicated in any pharmaceutical composition. Therefore the present invention also provides a pharmaceutical composition containing an optionally hydrated complex of ruthenium of Formula IV wherein Y^{IV} is a nitrogen-donor ligand selected from the group en, py, phen, bipy, cyclam and oep. Derivations of these ligands can be prepared by a skilled artisan and which will fall within the scope of the inventions.

Still further examples of metal complexes for use according to the present invention invlude optionally hydrated complexes of ruthenium or osmium of general Formula V

[M₁₋₃Y^{V}₁₋₁₈Cl₀₋₁₈]^{(0-6)±} Formula V

where Y^{V} is a combination of donor ligands such as are described hereinabove, for example ammine, dmso, oxalate, bipy, acac and methyl cyanide. Complexes of Formula V are present in for example

[Ru(NH₃)(dmso)₂Cl₃]

cis-[Ru(dmso)₄Cl₂]

cis-[Ru(NH₃)(dmso)₃Cl₂]

[Ru(dmso)₃Cl₃]

[Os(ox)(bipy)₂]H₂O

[Ru(acac)₂(MeCN)₂]CF₃SO₃

The complex ions of the latter two compounds above have not to the best of our knowledge been previously indicated in any pharmaceutical composition. Therefore the present invention also provides a pharmaceutical composition containing an optionally hydrated complex of formula [Os(ox)(bipy)₂]; and further a pharmaceutical composition containing an optionally hydrated complex of formula [Ru(acac)₂(MeCN)₂]⁺.

In use the complexes of the present invention may be included as an active component in a pharmaceutical composition containing an optionally hydrated complex of any of Formulae I-V, in admixture with a pharmaceutically acceptable carrier or diluent. Said pharmaceutical composition may be formulated according to well known principles, and may be in the form of a solution or suspension for parenteral administration in single or repeat doses or be in capsule, tablet, dragee, or other solid composition or as a solution or suspension for oral administration, or formulated into pessaries or suppositories, or sustained release forms of any of the above. The solution or suspension may be administered by a single or repeat bolus injection or continuous infusion, or any other desired schedule. Suitable diluents, carriers, excipients and other components are known. Said pharmaceutical composition may contain dosages determined in accordance with conventional pharmacological methods, suitable to provide active complexes in the dosage range in humans of 1 mg to 10 g per day and dosages in other mammals as determined by routine clinical veterinary practice. Actual required dosage is largely dependent on where in the body there is the excess concentration of NO or other reactive oxygen species and for how long overproduction continues or attenuation of the levels of NO or reactive oxygen species, where such reactive oxygen species is implicated in disease, is required.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 illustrates pressure changes induced by the compounds of the present invention, which reflect a reduction in available nitric oxide compared with control levels.

FIG. 2 shows the available nitric oxide concentration (micromoles/liter) following reaction of nitric oxide with compounds of the present invention as compared with control levels.

FIG. 3 demonstrates the inhibition of tumour growth by AMD6245 and AMD6221.

FIG. 4A-4G provides chemical structural formulas for the AMD-numbered compounds disclosed.

FIG. 5A-5C provides chemical structural formulas for the AMD-numbered compounds disclosed.

### DETAILED DESCRIPTION OF THE INVENTION

Introduction and General Description of the Invention

This invention is directed to metal complexes which are useful in binding nitric oxide with sufficiently high affinity as to make such complexes useful as pharmaceutical compositions for the treatment of diseases in mammals, preferably in the human body.

Some metal complexes are known in pharmaceutical compositions for the treatment of diseases in mammals, preferably in diseases of the human body. For example certain complexes of platinum and ruthenium have been used or indicated in the treatment of cancer. Metal complexes have not however been previously indicated in the treatment of disease relating to the overproduction of reactive oxygen species (including the overproduction of NO). This invention provides for the use of a neutral anionic or cationic metal complex having at least one site for coordination with NO of Formula I

[Mₐ(XbL)_{c}Y_{d}Zₑ]^{nt±} Formula I

in the manufacture of a medicament for the attenuation of NO levels and other reactive oxygen species when implicated in disease.

where:

M is a metal ion or a mixture of metal ions:

X is a cation or a mixture of cations:

L is a ligand, or mixture of ligands each containing at least two different donor atoms selected from the elements of Group IV, Group V or Group VI of the Periodic Table;

Y is a ligand or a mixture of the same or different ligands each containing at least one donor atom or more than one donor atom which donor atom is selected from the elements of Group IV, Group V or Group VI of the Periodic Table:

And

Z is a halide or pseudohalide ion or a mixture of halide ions and pseudohalide ions:

a=1-3; b=0-12; c=0-18; d=0-18; e=0-18; and n=O-10; provided that at least one of c, d and e is 1 or more.

And where c is 0: b is also 0;

And where a is 1: c, d and e are not greater than 9;

And where a is 2: c, d and e are not greater than 12.

By “complex” in this specification is meant a neutral complex or anionic or cationic species.

The term “Group” which is used herein is to be understood as a vertical column of the periodic table in which elements of each Group have similar physical and chemical properties. The definition of the Periodic Table is that credited to Mendeleev; Chamber Dictionary of Science and Technology, 1974 Published by W & R Chambers Ltd. The nomenclature of the compounds as disclosed herein are based upon common usage. The names of the compounds according to nomenclature of the American Chemical Abstracts Service (American Chemical Society) are also provided in Table 5.

This invention may also be stated as providing a method of attenuation of reactive oxygen species when implicated in diseases in mammals, preferably in diseases of the human body. Thus the invention comprises administering a pharmaceutical composition containing a neutral, anionic or cationic metal complex of Formula I.

This invention may also provide for the use of a neutral anionic or cationic metal complex of formula I in the manufacture of a medicament for the treatment of diseases in mammals, preferably in diseases of the human body in which reactive oxygen species are overproduced.

This invention may also be stated as providing a method of attenuation of nitric oxide when implicated in diseases in mammals, preferably in diseases of the human body. Thus the invention comprises administering a pharmaceutical composition containing a neutral, anionic or cationic metal complex of Formula I.

This invention may also be stated as providing a method of treatment of diseases of the human body resultant of overproduction of NO in the human body comprising administering a pharmaceutical composition containing a neutral anionic or cationic metal complex of formula I.

Where the formula I represents an anionic species a cation will also be present. Where formula I represent a cationic species an an ion will also be present. The metal complexes may be hydrated.

Preferably M is a first, second or third row transition metal ion. For example M may be an Rh, Ru, Os, Mn, Co, Cr or Re ion, and is preferably an Rh, Ru or Os ion.

Suitably M is in an oxidation state III. We have found surprisingly that when the metal ion for example ruthenium is in oxidation state III, the rate at which it binds with NO is significantly faster than when it is in oxidation state II.

X may be any cation, such as mono-, di- or tri-valent cation. Suitable cations may be H⁺, K⁺, Na⁺, NH₄⁺ or Ca²⁺. Conveniently X may be H⁺, K⁺, or Na⁺.

Preferably L is a polyaminocarboxylate ligand described herein by the general formulae A and B:

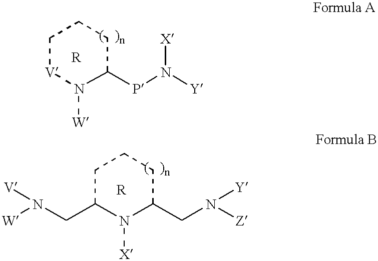

Where:

V′, W′, X′, Y′ and Z′ are independently selected from H, phenyl, heteroaryl, C₁₋₆alkyl, C₁₋₆alkylhydroxy, C₁₋₆alkylthiol, C₁₋₆alkylaryl, C, alkylheteroaryl, C₁₋₆alkylheterocyclyl and derivatives thereof. Preferred alkylheterocyclic groups are pyridinylmethylene, pyrazinylmethylene, pyrimidinylmethylene. The aromatic and heteroaromatic groups may be suitably substituted in single or multiple positions with halide, C₁₋₆alkyl, C₁₋₆alkoxy, C₁₋₆alkoxyaryl or benzyloxy, hydroxy, C₁₋₆hydroxyalkyl, thiol, carboxylic acid, carboxyalkylC₁₋₆, carboxamide, carboxamidoalkylC₁₋₆, anilide.

P′=CH₂, (CH₂)₂, CHOHCH₂,CH(OC₁₋₆alkyl)CH₂

V′, W′, X′, Y′ and Z′ may also be methylenecarboxylic acid, methylenecarboxyC₁₋₆alkyl, methylenecarboxamideC₁₋₆alkyl or heterocyclyl, methylenecarboxanilide, methylenecarboxamido derivatives of an aminoacid or peptide, methylenehydroxamic acid, methylene phosphonic acid, C₁₋₆alkylthiol.

In the above formulae, the ligands may be optionally fused with a heterocyclic ring R (n=0 or 1). Prefered heterocyclic groups are pyridine, pyrimidine, pyrazine, imidazole, thiazole, oxazole.

More preferably L is a ligand such as ethylenediamine-N,N′-diacetic acid (edda), ethylenediaminetetraacetic acid (edta), nitrilotriacetic acid (nta), dipicolinic acid (dipic), picolinic acid (pic), diethylenetri-aminepentaacetic acid (dtpa), thiobis(ethylenenitrilo)tetraacetic acid (tedta), dithioethanebis(ethylenenitrilo)tetraacetic acid (dtedta), and N-(2-hydroxethyl) ethylenediamine-triacetic acid (hedtra).

Preferably Y is a ligand containing nitrogen, oxygen, sulphur, carbon or phosphorus donor groups. Suitable nitrogen donor groups may be for example ammine, amine, nitrile and nitride or derivations thereof. Suitable oxygen donor groups may be for example carboxylic acid, ester or derivations thereof, water, oxide, sulphoxide, hydroxide, acetate, lactate, propionate, oxalate and maltolate. Suitable sulphur donor groups may be for example sulphoxide, dialkysulphide, dithiocarbamate or dithiophosphate. Suitable carbon donor groups may be for example carbon monoxide or isocyanide. Suitable phosphorus donor groups may be for example trialkylphosphine.

Z may be any halide and is preferably chloride, bromide or iodide. Most conveniently, Z is chloride.

Examples of metal complexes for use according to the present invention include optionally hydrated ruthenium complexes of Formula II

[Ru(H₀₋₆L^{II})₁₋₃Y₀₋₂Cl₀₋₄]^{(0-4)±} Formula II

where L^{II} is a

Preferably L is a polyaminocarboxylate ligand as already described herein by the general formulae A and B. More preferably, L is a polydentate aminocarboxylate ligand, for example edta, nta, dipic, pic, edda, tropolone, dtpa, hedtra, tedta or dtedta or diamide of edta or dtpa (or an amide or ester derivative thereof) or a mixture of any of these and Y is as defined above and may for example be selected from: acetylacetone (acac) a β-diketonate; water; dimethylsulphoxide (dmso); carboxylate; bidentate carboxylate; catechol; kojiic acid; maltol; hydroxide; tropolone; malonic acid; oxalic acid; 2.3-dihydroxynaphthalene; squaric acid; acetate; a sulphate and a glycolate. The skilled artisan will be able to substitute other known ligands at Y and which will fall within the scope of the inventions.

Preparative methods of tedta, dtedta and diamide of edta and dtpa are described in the following references respectively:

P Tse & J E Powell, Inorg Chem, (1985), 24,2727

G Schwartzenbach, H Senner, G Anderegg, Helv Chim Acta 1957, 40, 1886

M S Konings, W C Dow, D B Love, K N Raymond, S C Quay and S M Rocklage, Inorg Chem (1990), 29, 1488-1491

P N Turowski, S J Rodgers, R C Scarrow and K N Raymond, Inorg Chem (1988), 27, 474-481.

Where the complex of Formula II is an anion, a cation will be required. For example the complexes of Formula II are present in

K[Ru(Hedta)Cl]2H₂O

[Ru(H₂edta)(acac)]

K[Ru(hedtra)Cl]H₂O

K[Ru(dipic)₂]H₂O

(H₂pic)[RuCl₂(pic)₂](Hpic)H₂O

K[Ru(H₂edta)Cl₂]H₂O

K[Ru(Hnta)₂]½H₂O

K[Ru(H₂dtpa)Cl]H₂O

[Ru(Hhedtra)acac]H₂O

[Ru(Hhedtra)trop]

[Ru(H₃dtpa)Cl]

Complexes of formula II have not to the best of our knowledge been previously indicated in any pharmaceutical composition. Therefore the present invention also provides a pharmaceutical composition containing an optionally hydrated ruthenium complex of Formula II.

Further examples of metal complexes for use according to the present invention include optionally hydrated complexes of Formula III

[M₁₋₃Y₁₋₁₈Cl₀₋₁₈]^{(0-6)±} Formula III

where Y is a sulphur donor ligand. For example, such complex is present in

[Ru(mtc)₃] (mtc=4-morpolinecarbodithoic acid)

Ru(S₂CNCH₂CH₂NMeCH₂CH₂)₃½H₂O

Complexes of Formula III in which Y is a sulphur donor ligand have not to the best of our knowledge been previously indicated in any pharmaceutical composition. Therefore, the present invention also provides a pharmaceutical composition containing an optionally hydrated complex of Formula III when Y is a sulphur donor ligand.

Yet further examples of metal complexes for use according to the present invention include optionally hydrated complexes of ruthenium of Formula III

[M^{III}₁₋₃Y^{III}₁₋₁₈Cl₀₋₁₈]^{(0-6)±} Formula III

where M^{III} is ruthenium and Y^{III} is an oxygen-donor ligand such as acetate, lactate, water, oxide, propionate (COEt), oxalate (ox), or maltolate (maltol) or a combination of these. For example complexes of Formula III are present in

[Ru₃O(OAc)₆](OAc)

[Ru₃O(lac)₆](lac)

[Ru₂(OAc)₄]NO₃

[Ru₂(OCOEt)₄]NO₃

K₃[Ru(ox)₃]

[Ru₂(OAc)₄]Cl

[Ru(maltol)₃]

Some complexes of Formula III have not to the best of our knowledge been previously indicated in any pharmaceutical composition. Therefore the present invention also provides a pharmaceutical composition containing an optionally hydrated complex of ruthenium of Formula III wherein M^{III} is ruthenium and Y^{III} is an oxygen-donor ligand selected from the group acetate, lactate, oxide, propionate and maltolate.

Further examples of metal complexes for use according to the present invention include optionally hydrated complexes of ruthenium of Formula IV

[RuY^{IV}₁₋₉Cl₁₋₉]⁽0-4)± Formula IV

where Y^{IV} is a nitrogen-donor ligand such as: ammine; ethylenediamine (en); pyridine (py); 1,10-phenanthroline (phen): 2,2-bipyridine (bipy) or 1,4,8,11-tetraazacyclotetradecane (cyclam); 2,3,7,8,12,13,17,18-octaethylporphyrin (oep); or a combination of these. For example complexes of Formula IV are present in

[Ru(HN₃)₅Cl]Cl₂

[Ru(en)₃]I₃

trans-[RuCl₂(py)₄]

K[Ru(phen)Cl₄]

[Ru(cyclam)Cl₂]Cl

K[Ru(bipy)Cl₄]

[Ru(NH₃)₆]Cl₃

[Ru(NH₃)₄Cl₂]Cl

Ru(oep)Ph

Some complexes of Formula IV have not to the best of our knowledge been previously indicated in any pharmaceutical composition. Therefore the present invention also provides a pharmaceutical composition containing an optionally hydrated complex of ruthenium of Formula IV wherein Y^{IV} is a nitrogen-donor ligand selected from the group en, py, phen, bipy, cyclam and oep. Derivations of these ligands can be prepared by a skilled artisan and which will fall within the scope of the inventions.

Still further examples of metal complexes for use according to the present invention include optionally hydrated complexes of ruthenium or osmium of general Formula V

[M₁₋₃Y^{V}₁₋₁₈Cl₀₋₁₈]^{(0-6)±} Formula V

where Y^{V} is a combination of donor ligands such as are described hereinabove, for example ammine, dmso, oxalate, bipy, acac and methyl cyanide. Complexes of Formula V are present in for example

[Ru(NH₃)(dmso)₂Cl₃]

cis-[Ru(dmso)₄Cl₂]

cis-[Ru(NH₃)(dmso)₃Cl₂]

[Ru(dmso)₃Cl₃]

[Os(ox)(bipy)₂]H₂O

[Ru(acac)₂(MeCN)₂]CF₃SO₃

The complex ions of the latter two compounds above have not to the best of our knowledge been previously indicated in any pharmaceutical composition. Therefore the present invention also provides a pharmaceutical composition containing an optionally hydrated complex of formula [Os(ox)(bipy)₂]; and further a pharmaceutical composition containing an optionally hydrated complex of formula [Ru(acac)₂(MeCN)₂]⁺.

In use the complexes of the present invention may be included as an active component in a pharmaceutical composition containing an optionally hydrated complex of any of Formulae I-V, in admixture with a pharmaceutically acceptable carrier or diluent. Said pharmaceutical composition may be formulated according to well known principles, and may be in the form of a solution or suspension for parenteral administration in single or repeat doses or be in capsule, tablet, dragee, or other solid composition or as a solution or suspension for oral administration, or formulated into pessaries or suppositories, or sustained release forms of any of the above. The solution or suspension may be administered by a single or repeat bolus injection or continuous infusion, or any other desired schedule. Suitable diluents, carriers, excipients and other components are known. Said pharmaceutical composition may contain dosages determined in accordance with conventional pharmacological methods, suitable to provide active complexes in the dosage range in humans of 1 mg to 10 g per day. Actual required dosage is largely dependent on where in the body there is the excess concentration of NO or other reactive oxygen species and for how long overproduction continues or attenuation of the levels of NO or reactive oxygen species, where such reactive oxygen species is implicated in disease, is required. It will be understood that the present invention may be used in combination with any other pharmaceutical composition useful for this purpose.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein. Terms as used herein are based upon their art recognized meaning unless otherwise indicated and should be clearly understood by the ordinary skilled artisan.

### EXAMPLES

Having now generally described the invention, the same will be more readily understood through reference to the following examples which are provided by way of illustration, and are not intended to be limiting of the present invention, unless specified.

A number of commercially available compounds, and compounds prepared by routes known in the literature, containing the complexes of the present invention were tested in vitro, in vitro cell culture, and ex vivo in order to determine ability to coordinate with NO. The complexes tested were as follows:

TABLE 1

| Ex- | | |
|---|---|---|
| am- | | |
| ple | Compound | Literature Reference for Preparation |
| 1 | K[Ru(hedta)Cl]2H₂O | AA Diamantis & JV Dubrawski, |
| | | Inorg. Chem. (1981) 20:1142-50 |
| 2 | [Ru(H₂edta)(acac)] | AA Diamantis & JV Dubrawski, |
| | | Inorg. Chem. (1983) 22:1934-36 |
| 3 | K[Ru(hedtra)Cl]H₂O | HG Bajaj & R van Eldik, |
| | | Inorg. Chem. (1982) |
| | | 28:1980-3 |
| 4 | K[Ru(dipic)2]H₂O | NH Williams & JK Yandell, |
| | | Aust. J. Chem. (1983) |
| | | 36(12):2377-2386 |
| 5 | (H₂pic)[RuCCl₂(pic)₂] | JD Gilbert, D Rose & G Wilkinson, |
| | (Hpic)H₂O | J. Chem. Soc. (A) (1970):2765-9 |
| 6 | K[Ru(H₂edta)Cl₂]H₂O | AA Diamantis & JV Dubrawski, |
| | | Inorg. Chem. (1981) 20:1142-50 |
| 7 | K[Ru(hnta)₂]½H₂O | MM Taqui Khan, A Kumar & Z Shirin, |
| | | J. Chem. Research (M), (1986):1001-1009 |
| 8 | K[Ru(H₂dtpa)Cl]H₂O | MM Taqui Khan, A Kumar & Z Shirin, |
| | | J. Chem. Research (M), (1986):1001-1009 |
| 9 | [Ru₃O(lac)₆](lac) | A Spencer & G Wilkinson, J. Chem. |
| | | Soc. Dalton Trans (1972): 1570-77 |
| 10 | [Ru₃O(OAc)₆](OAc) | A Spencer & G Wilkinson, J. Chem. |
| | | Soc. Dalton Trans (1972): 1570-77 |
| 11 | [Ru₂(OAc)₄]NO₃ | M Mukaida, T Nomura & T Ishimori, |
| | | Bull. Chem. Soc. Japan (1972) 45:2143-7 |
| 12 | [Ru₂(OCOEt)₄]NO₃ | A Bino, FA Cotton & TR Felthouse, |
| | | Inorg. Chem. (1979) 18:2599-2604 |
| 13 | K₃[Ru(ox)₃] | CM Che, SS Kwong, CK Poon, TF Lai & |
| | | TCW Mak, Inorg. Chem. (1985) |
| | | 24:1359-63 |
| 14 | [Ru₂(OAc)₄]Cl | RW Mitchell, A Spencer & G Wilkinson, |
| | | J. Chem. Soc. Dalton Trans. |
| | | (1973) 846-54 |
| 15 | [Ru(NH₃)₅Cl]Cl₂ | AD Allen, F Bottomley, RO Harris, |
| | | VP Reinsalu & CV Senoff, |
| | | J. Amer. Chem. Soc. |
| | | (1967) 89:5595-5599 |
| 16 | [Ru(en)₃]I₃ | TJ Meyer & H Taube, Inorg. |
| | | Chem. (1968) 7:2369-2379 |
| 17 | K[RuCl₄(phen)]H₂O | BR James & RS McMillan, Inorg. Nucl. |
| | | Chem. Lett. (1975) 11(12):837-9 |
| 18 | [Ru(cyclam)Cl₂]Cl | PK Chan, DA Isabirye & CK Poon, Inorg. |
| | | Chem. (1975) 14:2579-80 |
| 19 | K[RuCl₄(bipy)] | BR James & RS McMillan, Inorg. Nucl. |
| | | Chem. Lett. (1975) 11(12):837-9 |
| 20 | [RuCl₃(dmso)₂(NH₃)] | Patent: International Publication |
| | | No. WO 91/13553 |
| 21 | [Ru(NH₃)₆]Cl₃ | Matthey Catalogue Sales: |
| | | Cat No [190245] |
| 22 | Cis-[RuCl₂(dmso)₄] | EA Alessio, G Mestroni, G Nardin, |
| | | WM Attia, M Calligaris, G Sava & |
| | | S Zorget, Inorg. Chem. (1988) |
| | | 27:4099-4106 |
| 23 | Cis-[RuCl₂(dmso)₃ | M Henn, E Alessio, G Mestrni, |
| | (NH₃)] | M Calligaris & WM Attia, Inorg. |
| | | Chim. Acta (1991) 187:39-50 |
| 24 | [RuCl₃(dmso)₃] | E Alessio, G Balducci, M Calligaris, |
| | | G Costa, WM Attia & G Mestroni, |
| | | Inorg. Chem. (1991) 30:609-618 |
| 25 | [Ru(mtc)₃] | AR Hendrickson, JM Hope & RL Martin, |
| | | J. Chem. Soc. Dalton Trans. |
| | | (1976) 20:2032-9 |
| 26 | [Ru(maltol)₃] | WP Griffith & SJ Greaves, Polyhedron |
| | | (1988) 7(10):1973-9 |
| 27 | [Ru(acac)₂ | Y Kasahara, T Hoshino, K Shimizu & |
| | (MeCN)₂]CF₃SO₃ | GP Sato, Chem. Lett. (1990) 3:381-4 |
| 28 | K₂[RuCl₅(H₂O)] | Matthey Catalogue Sales: |
| | | Cat No [190094] |
| 29 | [Os(ox)(bipy)₂].H₂O | DA Buckingham, FP Dwyer, |
| | | HA Goodwin & AM Sargeson, Aust. |
| | | J. Chem. (1964) 325-336 GM Bryant, |
| | | JE Fergusson & HKJ Powell, Aust. J. |
| | | Chem. (1971) 24(2):257-73 |
| 30 | [Ru(NH₃)₄Cl₂]Cl | SD Pell, MM Sherban, V Tramintano & |
| | | MJ Clarke, Inorg Synth (1989) 26:65 |
| 31 | [Ru(Hedtra)(dppm)] | MM Taqui Khan, K Venkatasubramanian, |
| | | Z Shirin, MM Bhadbhade, J Chem Soc |
| | | Dalt Trans (1992) 885-890 |
| 32 | Ru(oep)Ph | M Ke, SJ Rettig, BR James & D Dolphin, |
| | | J Chem Soc Chem Commun (1987) 1110 |

A number of new compounds were prepared according to the following protocols. The first four compounds are examples of rutheniuim complexes of formula [Ru(H₀₋₆L^{II})₁₋₃Y₀₋₂Cl₀₋₄]^{(0-4)±} (Formula II), the subsequent two are examples of [M₁₋₃Y₁₋₈Cl₀₋₁₈]^{(0-6)±} (formula III).

Preparation of [Ru(Hhedtra)acac].H₂O

Excess acetylacetone (1 cm³) was added to an aqueous solution (5 cm³) of K[Ru(hedtra)Cl] (0.5 g). The solution color changed to violet. The mixture was warmed for 20 minutes then left to stand at room temperature for 20 minutes. The violet solution was extracted with chloroform (20 cm³). The extraction was repeated twice more. A violet product precipitated from the aqueous fraction. The product was filtered, washed in acetone and dried in vacuo, yield 0.1 g (18%).

Anal. Calc. For C₁₅H₂₅O₁₀N₂Ru: C, 36.43; H, 5.11; N, 5.70. Found: C, 36.16; H, 5.42; N, 5.61%.

Preparation of [Ru(Hhedtra)trop]2H₂O

A three-fold excess of tropolone (0.78 g) dissolved in 50:50 water/absolute ehtnaol (5 cm³) was added to a warm aqueous solution of K[Ru(hedtra)Cl] (10 cm³). The mixture was heated for 1 hour. On cooling, the dark green mixture was extracted with 3×20 cm³ portion sof dichloromethane. On standing, a dark green product precipitated from the aqueous fraction. The product was filtered, washed with water (1 cm³), ether and dried in vacuo, yield 0.4 g (36%).

Anal. Cal. For C₁₇H₂₂N₂O₉Ru.2H₂O: C, 38.13; H, 4.86; N, 5.23. found: C, 38.55; H, 4.67; N, 5.28%.

Preparation of [Ru(H₃dtpa)CI]

K₂[RuCl₅H₂O].xH₂O (1 g) was suspended in HClO₄ (15 cm³, 1 mM) and diethylenetriaminepentaacetic acid (1.05 g) added. The reaction mixture was heated under reflux for 1.5 hours forming a yellow/brown solution. On cooling a yellow product crystallised which was collected by filtration, washed with 90% absolute ethanol/water, diethyl ether and dried in vacuo, yield 0.75 g, 53%.

Anal. calcd. for C₁₄H₂₁N₃O₁₀ClRu: C, 31.85; H, 3.98; N, 7.96; Cl, 6.73. Found: C, 29.77; H, 3.81; N, 7.36; Cl, 6.64.

Preparation of K[RuHHBEDCl]3H₂O

0.41 g of K₂[RuCl₅]xH₂O was dissolved in water (20 ml). To this solution was added 1 equivalent (0.39 g) of N,N′di(2-hydroxy-benzyl)ethylene-diamine N,N-diacetic acid (hbed) dissolved in water (50 ml) with KOH (0.12 g) and MeOH (1 ml). This mixture was heated at reflux for 90 minutes. Upon cooling a dark, insoluble precipitate formed. This material was removed by filtration and the resulting red-violet solution was taken to dryness by rotary evapouration. Trituration with water and washing with acetone yilede 90 mg of a dark solid.

Anal. Calcd. for C₁₈H₂₂N₂O₉RuClK: C, 36.89; H, 3.96; N, 4.78; Cl, 6.04. Found: C, 37.09; H, 4.23; N, 4.92; Cl, 6.28.

Preparation of Ru(S₂CNCH₂CH₂NMeCH₂CH₂)₃½H₂O

Me₄N[S₂CNCH₂CH₂NMeCH₂CH₂] was made by the standard method and crystallised from methanol-ether in 71% yield.

RuCl₃xH₂O, 0.50 g, 2.15 mmol was refluxed in 30 ml of methanol for 10 minutes and cooled. 1.87 g, 7.50 mmol of Me₄N[S₂CNCH₂CH₂NMeCH₂CH₂] was added and the mixture refluxed for 16 hours. After cooling 0.72 g of crude product was filtered off, dissolved in dichloromethane and filtered. The filtrate was loaded into 15 cc of basic alumina and eluted with dichloromethane. Removal of solvent and crystallisation from dichloromethane with ether by vapour-phase diffusion gave 0.51 g, 0.80 mmol, 37% of brown-black crystals, Ru(S₂CNCH₂CH₂NMeCH₂CH₂)₃½H₂O.

Analysis for C₁₈H₃₄N₆O_{0.5}RuS₆: Calc: C, 34.00; H, 5.39; N, 13.22; S, 30.25. Found: C, 34.21; H, 5.47; N, 13.12; S, 30.36.

Preparation of Ru[S₂P(OC₂H₂OC₂H₄OMe)₂]₃

K[S₂P(OC₂H₄OC₂H₄OMe)₂]₃ was made by standard method and crystallised from methanol in 76% yield.

RuCl₃xH₂O, 1.00 g, 4.30 mmol was refluxed in 50 ml of 0.1 N HCl with 1 ml of ethanol for 20 minutes and cooled. To this solution was added 5.28 g (excess) K[S₂P(OC₂H₄OC₂HROMe)₂] and the mixture stirred at 30° C. for 1 hour. The reaction mixture was extracted with dichloromethane and the solvent removed. The residue was extracted with ether-hexane and solvents removed. This residue was crystallised from 25 ml of hot ether by cooling to −20° C. giving 2.98 of red crystals. 2.41 g of the crude product was purified by chromatography on 60 cc of silica gel with 5% ethanol in ether. The first band was collected, reduced to dryness and crystallised from ether by cooling to −20° C. The yield of red crystals, Ru(S₂P[OC₂H₄OC₂H₄OMe]₂)₃, was 2.16 g, 56%.

Analysis for C₃₀H₆₆O₁₈P₃RuS₆: Calc: C, 32.72; H, 6.04; S, 17.47. Found: C, 32.68; H, 6.08; S, 17.16.

In the in vitro tests, which were carried out in an atmosphere of argon, each compound (1×104 moles) was dissolved in double-distilled deionized and deoxygenated water. The resulting solution was placed in a three-necked pear-shaped flask and stirred by a magnetic stirrer at constant speed of 1000 rpm, at a constant temperature in the range 20° C.-24° C. A manometer was attached to the flask, and purified, dried nitric oxide gas (known volume in the range 3-5cm³) was introduced via a septum, using a gas syringe, at atmospheric pressure into the headspace above the reaction solution. The pressure within the flask was recorded periodically over a period of one hour.

A control experiment was carried out according to the above but without any complex present.

The recorded pressures in association with the results of the control experiment were analysed in order to determine the rate of NO uptake as a finction of time for each compound tested.

On completion of each in vitro test, the reaction solution was freeze-dried. An infrared spectrum of the freeze-dried product provided information on metal-NO bond formation.

In the in vitro cell culture tests, murine (RAW264) macrophage cell lines, which can be induced to produce nitric oxide, were seeded, 10⁶ cells/well, onto 24 well culture plates of 2 ml volume per well, in Eagles modified minimal essential medium (MEM) plus 10% fetal bovine serum without phenol red.

The cells were activated to produce nitric oxide, with 10 μg/ml lipopolysaccharide and 100 units/ml interferon γ for 18 hours. Concurrently, test compounds made up in MEM were added at non-cytotoxic concentrations. Control cells as above, which were activated to produce nitric oxide as above, but to which no test compound was added, were used as a measure of the amouint of nitric oxide produced by the cells during the tests. (See S. P. Fricker, E. Slade, N. A. Powell, O. J. Vaughan, G. R. Henderson, B. A. Murrer, I. L. Megson, S. K. Bisland, F. W. Flitney, Ruthenium complexes as nitric oxide scavengers: a potential therapeutic approach to nitric oxide-mediated diseases, Br. J Pharmacol., 1997, 122, 1441-1449.)

Background nitric oxide was assessed by measurement of nitrate and nitrite in cells which were not activated.

Cell viability was confirmed by Trypan blue dye exclusion at the end of the incubation period.

Nitric oxide was determined by measurement of nitrate and nitrite in the cell supernatant. These anions are the stable end-products of reactions of NO in solution. Such reactions may or may not be catalysed in biological systems. The sum of nitrite and nitrate concentrations gives the total NO production. Nitrite was determined using the Griess reaction in which nitrite reacts with 1% sulphanilamide in 5% H₃PO₄/0.1% naphthylethylenediamine dihydrochloride to form a chromophore absorbing at 540 nm. Nitrate was determined by reducing nitrate to nitrite with a bacterial nitrate reductase from Pseudomonas oleovorans and then measuring nitrite with the Griess reaction. In the absence of test compounds nitrite concentration plus nitrate concentration is equal to total nitric oxide production. The effect of test compounds on available nitric oxide (measured as nitrite+nitrate) was determined. The reduction in available nitric oxide compared with the control level may be taken as an indication of the degree of binding of NO by the test compounds.

In the ex vivo tests, segments of rat tail artery (0.8-1.5 cm) were dissected free from normotensive adult Wistar rats. The arteries were internally perfused with Krebs solution (mM: NaCl 118, KCl 4.7, NaHCO₃ 25, NaH₂PO₄ 1.15, CaCl₂2.5, MgCl₂ 1.1, glucose 5.6 and gassed with 95% O₂/5% CO₂ to maintain a pH of 7.4) in a constant flow perfusion apparatus. A differential pressure transducer located upstream of the vessel detected changes in back pressure. The rat tail artery preparation was pre-contracted with 6.5 μM phenylephrine to give a physiologically normal pressure of 100-120 mm Hg. The pre-contracted vessels were then perfused with the test compound. The arteries were perfused with Krebs solution between applications of test compound to wash out the test compound.

Pressure changes in the system served to indicate artery vasoconstriction. The vasoconstriction is a direct result of the removal of endogenous nitric oxide (edrf) from the endothelial cells of the rat tail artery.

Results

The results of the in vitro, in vitro cell culture and ex vivo tests were as follows:

In Vitro Tests

### EXAMPLE 1

### K[Ru(hedta)Cl]2H₂O

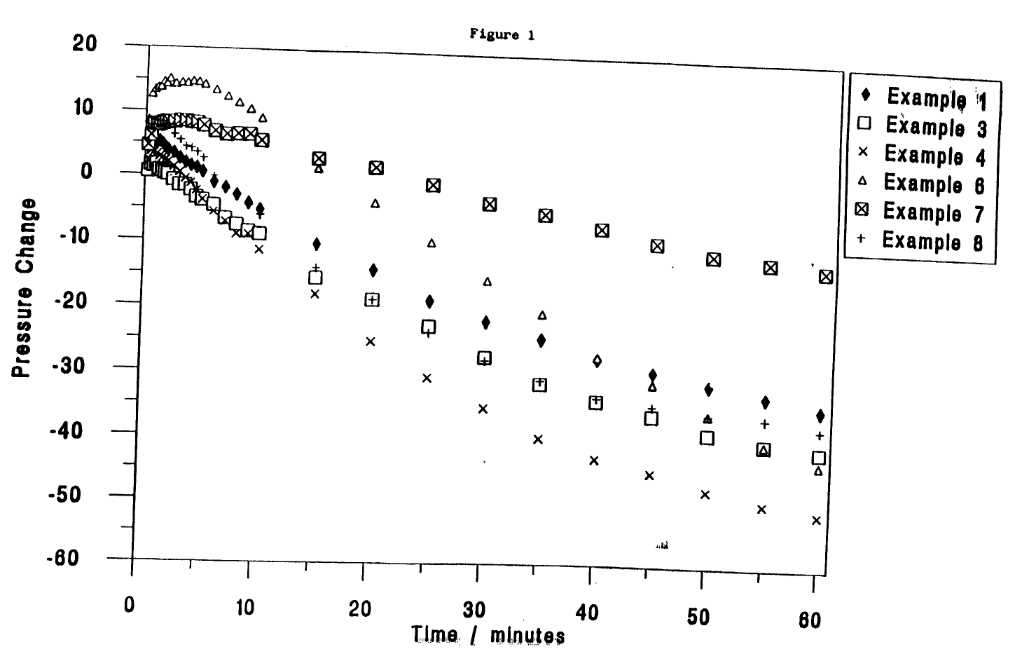

A pressure decrease indicated binding of NO to the metal compound. This is illustrated in FIG. 1.

The IR spectrum showed a peak at 1897 cm⁻¹, indicating the presence of a Ru-NO bond.

### EXAMPLE 2

### [Ru(H₂edta)(acac)]

The IR spectrum showed a peak at 1896 cm⁻¹, indicating the presence of a Ru-NO bond.

### EXAMPLE 3

### K[Ru(hedtra)Cl]H₂O

A pressure decrease indicated binding of NO to the metal compound. This is illustrated in FIG. 1.

The IR spectrum showed a peak at 1889 cm⁻¹, indicating the presence of a Ru-NO bond.

### EXAMPLE 4

K[Ru(dipic)₂H₂O

A pressure decrease indicated binding of NO to the metal compound. This is illustrated in FIG. 1.

The IR spectrum showed a peak at 1915 cm⁻¹, indicating the presence of a Ru-NO bond.

### EXAMPLE 5

### (H₂pic)[RuCl₂(pic)₂](Hpic)H₂O

The IR spectrum showed a peak at 1888 cm⁻¹, indicating the presence of a Ru-NO bond.

### EXAMPLE 6

### K[Ru(H₂edta)Cl₂]H₂O

A pressure decrease indicated binding of NO to the metal compound. This is illustrated in FIG. 1.

The IR spectrum showed a peak at 1896 cm⁻¹, indicating the presence of a Ru-NO bond.

### EXAMPLE 7

### K[Ru(Hnta)₂]½H₂O A pressure decrease indicated binding of NO to the metal compound. This is illustrated in FIG. 1.

The IR spectrum showed a peak at 1889 cm⁻¹, indicating the presence of a Ru-NO bond.

### EXAMPLE 8

### K[Ru(H₂dtpa)Cl]H₂O

A pressure decrease indicated binding of NO to the metal compound. This is illustrated in FIG. 1.

The IR spectrum showed a peak at 1905 cm⁻¹, indicating the presence of a Ru-NO bond.

### EXAMPLE 9

### [Ru₃O(lac)₆](lac)

The IR spectrum showed a peak at 1884 cm⁻¹, indicating the presence of a Ru-NO bond.

### EXAMPLE 10

### [Ru₃O(OAc)₆](OAc)

The IR spectrum showed a peak at 1877 cm⁻¹, indicating the presence of a Ru-NO bond.

### EXAMPLE 11

### [Ru₂(OAc)₄]NO₃

The IR spectrum showed a peak at 1891 cm⁻¹, indicating the presence of a Ru-NO bond.

### EXAMPLE 12

### [Ru(OCOEt)₄]NO₃

The IR spectrum showed a peak at 1891 cm⁻¹, indicating the presence of a Ru-NO bond.

### EXAMPLE 13

### K₃[Ru(ox)₃]

The IR spectrum showed a peak at 1889 cm⁻¹, indicating the presence of a Ru-NO bond.

### EXAMPLE 14

### [Ru₂(OAc)₄]Cl

The IR spectrum showed a peak at 1895 cm⁻¹, indicating the presence of a Ru-NO bond.

### EXAMPLE 15

### [Ru(NH₃)₅Cl]Cl₂

The IR spectrum showed two peaks at 1909 cm⁻¹ and 1928 cm⁻¹, indicating the presence of a Ru-NO bond.

### EXAMPLE 16

### [Ru(en)₃]I₃

The IR spectrum showed a peak at 1906 cm⁻¹, indicating the presence of a Ru-NO bond.

### EXAMPLE 17

### K[RuCl₄(phen)]H₂O

The IR spectrum showed a peak at 1904 cm⁻¹, indicating the presence of a Ru-NO bond.

### EXAMPLE 18

### [Ru(cyclam)Cl₂]Cl

The IR spectrum showed a peak at 1895 cm⁻¹, indicating the presence of a Ru-NO bond.

### EXAMPLE 19

### K[RuCl₄(bipy)]

The IR spectrum showed a peak at 1885 cm⁻¹, indicating the presence of a Ru-NO bond.

### EXAMPLE 20

### [RuCl₃(dmso)₂(NH₃)]

The IR spectrum showed a peak at 1889 cm⁻¹, indicating the presence of a Ru-NO bond.

### EXAMPLE 21

### [Ru(NH₃)₆]Cl₃

The IR spectrum showed a peak at 1910 cm⁻¹, indicating the presence of a Ru-NO bond.

### EXAMPLE 22

### cis-[RuCl₂(dmso)₄]

The IR spectrum showed a peak at 1881 cm⁻¹, indicating the presence of a Ru-NO bond.

### EXAMPLE 23

### cis-[RuCl₂(dmso)₃(NH₃)]

The IR spectrum showed a peak at 1893 cm⁻¹, indicating the presence of a Ru-NO bond.

### EXAMPLE 24

### [RuCl₃(dmso)₃]

The IR spectrum showed a peak at 1880 cm⁻¹, indicating the presence of a Ru-NO bond.

### EXAMPLE 25

### [Ru(mtc)₃]

The IR spectrum showed a peak at 1862 cm⁻¹, indicating the presence of a Ru-NO bond.

### EXAMPLE 26

### [Ru(maltol)₃]

The IR spectrum showed a peak at 1866 cm⁻¹, indicating the presence of a Ru-NO bond.

### EXAMPLE 27

### [Ru(acac)₂(MeCN)₂](CF₃SO₃)

The IR spectrum showed a peak at 1899 cm⁻¹, indicating the presence of a Ru-NO bond.

### EXAMPLE 28

### K₂[RuCl₅(H₂O)]

The IR spectrum showed a peak at 1903 cm⁻¹, indicating the presence of a Ru-NO bond.

### EXAMPLE 29

### [Os(ox)(bipy)₂]H₂O

The IR spectrum showed a peak at 1894 cm⁻¹, indicating the presence of a Ru-NO bond.

In Vitro Cell Culture Tests

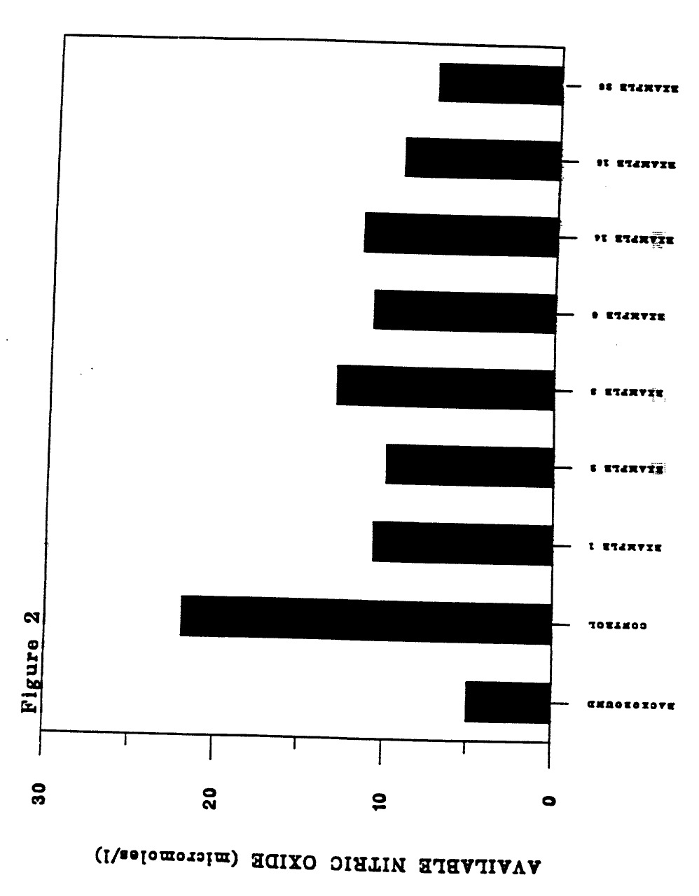

Results are shown in Table 2 and FIG. 2 for the in vitro cell culture tests using the compounds of Examples: 1-3, 6 14, 15 and 26, as follows.

### EXAMPLE 1

### K[Ru(Hedta)Cl]2H₂O

Available nitric oxide was reduced in a dose-dependent fashion with a maximum reduction of 75% at a concentration of 100 μM.

### EXAMPLE 2

### [Ru(H₂edta)(acac)]

Available nitric oxide was reduced by 82% at 100 μM test compound.

### EXAMPLE 3

### K[Ru(Hedtra)Cl]H₂O

Available nitric oxide was reduced by 42% at 100 μM.

### EXAMPLE 6

### K[Ru(H₂edta)Cl₂]H₂O

Available nitric oxide was reduced by 77% at 100 μM test compound.

### EXAMPLE 14

### [Ru₂(OAc)₄]Cl

Available nitric oxide was reduced by 47% at 100 μM.

### EXAMPLE 15

### [Ru(NH₃)₅Cl]Cl₂

Available nitric oxide was reduced by 86% at 100 μM test compound.

### EXAMPLE 26

### [Ru(maltol)₃]

Available nitric oxide was reduced by 71% at 100 μM.

TABLE 2

| | | % Decrease of Available |
|---|---|---|
| | | Nitric Oxide |
| Example 1 | 25 μM | 12 |
| | 50 μM | 23 |
| | 100 μM | 75 |
| Example 2 | 100 μM | 82 |
| Example 3 | 100 μM | 42 |
| Example 6 | 100 μM | 77 |
| Example 14 | 100 μM | 47 |
| Example 15 | 100 μM | 86 |
| Example 26 | 100 μM | 71 |

Ex Vivo Tests

Results are shown in Table 3 for the ex vivo tests using the compounds of Examples: 2, 3, 14, 15 and 26, as follows.

### EXAMPLE 2

Application of test compound resulted in a dose-dependent vasoconstriction at 10 μM and 100 μM. This effect was reversible by washout with Krebs solution.

### EXAMPLE 3

Application of test compound resulted in a dose-dependent vasoconstriction at 10 μM and 100 μM. This effect was reversible by washout with Krebs solution.

### EXAMPLE 14

Application of test compound resulted in a dose-dependent vasoconstriction at 10 μM and 100 μM. This effect was reversible by washout with Krebs solution.

### EXAMPLE 15

Application of test compound resulted in a dose-dependent vasoconstriction at 10 μM and 100 μM. This effect was reversible by washout with Krebs solution.

### EXAMPLE 26

Application of test compound resulted in a dose-dependent vasoconstriction at 10 μM and 100 μM and 1000 μM. This effect was reversible by washout with Krebs solution.

TABLE 3

| | | % Vasoconstriction |
|---|---|---|
| Example 2 | 10 μM | 20 |
| | 100 μM | 69 |
| Example 3 | 10 μM | 17 |
| | 100 μM | 59 |
| Example 14 | 10 μM | 11 |
| | 100 μM | 40 |
| Example 15 | 10 μM | 77 |
| | 100 μM | 86 |
| Example 26 | 10 μM | 10 |
| | 100 μM | 18 |
| | 1000 μM | 25 |

Experimental

### EXAMPLE 33

### AMD7040: Synthesis of the Ru(III) complex of N,N′-[2,6-pyridylbis(methylene)]bis-iminodiacetic acid (pbbida)

N,N′-[2,6-pyridylbis(methylene)]bis-iminodiacetic acid (Na₃Hpbbida)

An aqueous solution of sodium hydroxide (30 mL, 0.01 M), 2,6-dibromomethylpyridine.HBr (1.0 g, 2.9 mmol), iminodiacetic acid dimethyl ester (0.934 g, 5.8 mmol), and cetyltrimethylammonium bromide (0.21 g, 0.58 mmol) was stirred at room temperature for 3 days. A white precipitate formed which was removed by filtration and the filtrate was evaporated to give a white solid. This solid was purified by re-crystallisation from water and ethanol to give the desired compound as the tri-sodium salt (0.9 g, 71%). ¹H NMR (D₂O) δ 3.27 (s, 8H), 3.93 (s, 4H), 7.30 (d, 2H, J=7.5 Hz), 7.80 (t, 1H, J=7.8 Hz).

Preparation of [Ru(H₂pbbida)Cl].2.5H₂O.

[Dihydrogen chloro[[2,6-(pyridinyl-κN)methyl]bis[N-(carboxymethyl)glycinato-κN,κO]] ruthenium (III)]

Na₃Hpbbida (0.78 g, 1.8 mmol) was dissolved in HCl (20 mL, 1 mM) and the pH was adjusted to pH 4 with 1N HCl. K₂[RuCl₅(OH₂)] (0.67 g, 1.8 mmol) dissolved in a minimum amount of aqueous HCl (1 mM) was added to the ligand solution and the resulting mixture was heated to reflux for 1.5 hours. A yellow precipitate formed throughout the course of the reaction. The reaction mixture was cooled in an ice bath and the yellow solid was collected via filtration, washed with ice cold water, ethanol and diethyl ether and then dried in vacuo at 70° C. for 2 hours (0.55 g, 56%). IR (CSI) ν(cm⁻¹) 1734(CO₂₋) 1649(CO₂₋) coordinated).

Anal. Calcd. for C₁₅H₁₇ClN₃O₈Ru.2.5H₂O: C, 32.82; H 4.04; N, 7.66; Cl, 6.47. Found: C, 32.82; H, 3.95; N 7.66; Cl, 6.47.

### EXAMPLE 34

### AMD7043: Synthesis of the Ru(III) complex of N,N′-bis[2-pyridyl(methylene)]ethylenediamine-N,N′-diacetic acid (H₂bped)

The ligand, H₂bped, was prepared according to literature procedures: See P. Caravan, S. J. Rettig, C. Orvig. Inorg. Chem. 1997, 36, 1306.

Preparation of [Ru(H₂bped)Cl₂]Cl

[Dihydrogen dichloro[[N,N′-1,2-ethanediyl]bis[(2-pyridinyl-κN)methylglycinato-κN]ruthenium (III) chloride]

H₂bped.2HCl (1.0 g, 2.5 mmol) was dissolved in HCl (25 mL, 1 mM) and the pH was adjusted to pH 4 with 1N NaOH. A solution of K₂[RuCl₅(OH₂)] in HCl (minimum volume, 1 mM) was added to the ligand solution and the reaction mixture was heated to reflux for 1.5 hours. The dark green solution was reduced to approximately one half the original volume and on slow evaporation a yellow-orange solid precipitated from the reaction mixture. This was collected by filtration and re-crystallised from H₂O/EtOH to yield an orange micro-crystalline solid (0.37 g, 26%).

IR (CSI) ν(cm⁻¹)1726(CO₂₋). Anal. Calcd. for C₁₈H₂₂Cl₃N₄O₄Ru: C, 38.21; H, 3.92; N, 9.90; Cl, 18.80. Found: C, 38.21; H, 3.96; N 9.90; Cl, 18.79.

### EXAMPLE 35

### AMD7056: Synthesis of the Ru(III) complex of N-[2-(2-pyridylcarboxamido)ethyl]iminodiacetic (pceida).

To a stirred solution of N-BOCethylenediamine (0.462 g) in dioxane (10 mL) was added picolinic acid hydroxysuccinimdyl ester (0.635 g) and the mixture was allowed to stir overnight. The reaction mixture was filtered and the filtrate was diluted with dichloromethane and washed with saturated aqueous sodium carbonate and then brine. The organic layer was dried (Na₂SO₄) and then evaporated to give a white solid (0.691 g, 90%). This was used without further purification.

The solid from above (0.691 g) was dissolved in pre-cooled (0° C.) trifluoroacetic acid (5 mL). The mixture was stirred for 2 hours at 0° C. and then room temperature for 15 minutes. The mixture was evaporated to dryness to give the pyridyl amine intermediate (˜quantitative). The residue was dissolved in DMF (20 mL) with stirring and K₂CO₃ (1.8 g, 5.0 equiv.) followed by t-butyl bromoacetate (0.84 mL, 2.1 equiv.) were added and the reaction mixture was allowed to stir at room temperature for six days. The reaction mixture was diluted with water and extracted with ethyl acetate. The combined organic phases were then washed with brine and water, dried (MgSO₄) and evaporated to give the desired bis-t-butyl ester (1.02 g, 100%) as a light yellow oil.

¹H NMR (CDCl₃) δ 1.42 (s, 9H), 1.45 (s, 9H), 3.00 (t, 2H, J=6.1 Hz), 3.48 (s, 2H), 3.50-3.60 (m, 2H), 7.40 (m, 2H), 7.82 (dt, 1H, J=7.8, 1.6 Hz), 8.19 (d, 1H, J=7.8 Hz), 8.59 (d, 1H, J=4.6 Hz), 8.70 (br. m, 1H).

N-[2-(2-pyridylcarboxamido)ethyl]iminodiacetic·TFA salt (H₂pceida·TFA).

The di-t-butyl ester (1.02 g) from above was dissolved in dichloromethane (1 mL) and cooled to 0° C. Pre-cooled trifluoroacetic acid was added (7 mL) and the solution was allowed to stir overnight at room temperature. The reaction mixture was then evaporated and the residue was dissolved in water (10 mL) and lyophilised to give the desired ligand (pceida) as a light yellow solid (0.71 g, 69%).

¹H NMR (D₂O) δ 3.53 (t, 2H, J=5.7 Hz), 3.85 (t, 2H, J=5.7 Hz), 3.90 (s, 2H), 7.65 (m, 1H), 7.95-8.10 (m, 2H), 8.65 (s, 1H, J=4.8 Hz). Anal Calcd. for C₁₂H₁₅N₃O₅₋.TFA₋.H₂O: C, 40.69; H, 4.39; N, 10.17. Found: C, 40.84; H, 4.32; N, 9.99.

Preparation of [Ru(pceida)(OH₂)Cl.1.5H₂O

[Aquachloro[[N-2-[(2-pyridinyl-κN)oxo-methyl)aminoethyl][((2-carboxy-κO)methyl)glycinato-κN,κO]] ruthenium (III)]

H₂pceida.TFA (0.4 g, 1 mmol) and K₂[RuCl₅(OH₂)] (0.38 g, 1 mmol) were dissolved in de-ionised water (10 mL) and the pH adjusted to pH5 with 1N NaOH. KCl (0.075 g, 1 mmol) was added to the reaction mixture and the solution was heated to reflux for 3 hours. The solution was cooled to room temperature and subsequently in an ice bath. Upon cooling a dark red-orange precipitate formed which was collected by filtration, washed with ice cold water and dried in vacuo at 40° C. overnight.

Yield: 0.13 g, 29%. IR (CSI) ν(cm⁻¹) 1649(CO₂₋). Anal. Calcd. for C₁₂H₁₅ClN₃O₆Ru.1.5H₂O: C, 31.28; H, 3.94; N, 9.12; Cl, 7.69. Found: C, 31.43; H, 3.92; N, 9.05; Cl, 7.80.

### EXAMPLE 36

### AMD7046: Synthesis of the Ru(III) complex of N-[2-pyridyl(methylene)]ethylenediamine-N,N′,N′-triacetic acid (pedta).

To a solution of 2-pyridinecarboxaldehyde (3.2 g, 0.03 mol) in benzene (50 mL) was added N-BOC ethylenediamine (5.26 g, 1.1 equiv.) and the mixture was heated to reflux with stirring in a Dean-Stark apparatus for 1.5 hours. The reaction mixture was evaporated to dryness, dissolved in methanol (50 mL) and 5% palladium on carbon was added (0.5 g). The mixture was hydrogenated at 50 psi on a Parr apparatus overnight. The mixture was filtered through celite, and the filtrate was evaporated to give the pyridine intermediate (˜quantitative).

¹H NMR (CDCl₃) δ 1.40 (s, 9H), 2.75-2.85 (m, 2H), 3.20-3.35 (m, 2H), 3.90 (s, 2H), 5.30 (br. S, 1H), 7.10-7.20 (m, 1H), 7.30-7.36 (m, 1H), 7.60-7.70 (m, 1H), 8.50-8.60 (m, 1H).

To a stirred solution of the pyridine intermediate from above (5.08 g) in dichloromethane (30 mL) was added trifluoroacetic acid (30 mL) and the mixture was allowed to continue stirring overnight at room temperature. The mixture was evaporated to give a dark oil.

¹H NMR (d₆-DMSO/D₂O) δ 3.10-3.20 (m, 2H), 3.20-3.30 (m, 2H), 4.48 (s, 2H), 7.40-7.45 (m, 2H), 7.80-7.90 (m, 1 H), 8.60 (m, 1H). This intermediate was used without further purification in the next step.

N-[2-pyridyl(methylene)]ethylenediamine-N,N′,N′-triacetic acid tri-t-butyl ester

To a solution of the oil from above in DMF (˜80 mL) was added K₂CO₃ (27.9 g, 10.0 equiv.) followed by t-butylbromoacetate (8.95 mL, 3.0 equiv.) and the mixture was allowed to stir at room temperature for 48 hours. The reaction mixture was filtered through celite and the filtrate was evaporated to give a dark oil. Purification by column chromatography on silica gel (5% MeOH/CH₂Cl₂) gave the tri-t-butyl ester (4.14 g, 42% for two steps) as a light yellow oil.

¹H NMR (CDCl₃) δ 1.35-1.50 (m, 27H), 2.83-2.86 (m, 4H), 3.37 (s, 2H), 3.43 (s, 4H), 3.95 (s, 2H), 7.10-7.20 (m, 1H), 7.52 (d, 1H, J=7.5 Hz), 7.64 (dt, 1H, J=7.5, 1.7 Hz), 8.51 (d, 1H, J=4.7 Hz).

N-[2-pyridyl(methylene)lethylenediamine-N,N′,N′-triacetic acid.TFA salt (pedta)

The tri-t-butyl ester from above (4.14 g) was dissolved in CH₂Cl₂ (20 mL) with stirring and trifluoroacetic acid (30 mL) was added in one portion. The mixture was allowed to stir at room temperature overnight and was then evaporated. The residue was dissolved in water (˜40 mL) and charcoal (550 mg) was added. The mixture was heated to 70° C. and filtered through celite and the combined filtrates were then evaporated to small volume and lyophilised to give the desired ligand (pedta) as a yellow solid (3.24 g, 73%).

¹H NMR (D₂O) δ 3.00-3.15 (m, 2H), 3.20-3.30 (m, 2H), 3.59 (s, 4H), 4.04 (s, 2H), 4.51 (s, 2H), 7.50 (m, 1H), 7.61 (d, 1H, J=7.7 Hz), 7.98 (dt, 1H, J=7.7, 1.6 Hz), 8.63 (d, 1H, J=5.0 Hz). Anal. Calcd. for C₁₄H₁₉N₃O₆₋.1.8TFA: C, 39.83; H, 3.95; N, 7.92. Found: C, 38.85; H, 4.19; N, 8.06.

Preparation of [Ru(Hpedta)Cl].0.5H₂O

[Hydrogen chloro[N-[bis((2-(carboxy-κO)methyl)imino-κN)ethyl]-(2-pyridinyl-κN)methylglycinato-κN]ruthenium (III)].

H₃pedta.TFA (0.75 g, 1.3 mmol) was dissolved in HCl (1.5 mL, 1 mM). A solution of K₂[RuCl₅(OH₂)] (0.5 g, 1.3 mmol) in HCl (2 mL, 1 mM) was added to the ligand solution. The reaction mixture was heated to reflux for 2 hours and subsequently cooled to room temperature. An orange solid precipitated from the solution, which was collected by filtration, washed with ethanol and diethyl ether, and dried in vacuo at 40° C. overnight (0.26 g, 43%). IR (CSI) ν(cm⁻¹) 1730(CO₂H); 1688, 1618 (CO₂) coordinated).

Anal. Calcd. for C₁₄H₁₇ClN₃O₆Ru.0.5H₂O: C 35.87; H 3.87; N 8.96; Cl 7.56. Found: C, 35.86; H, 3.79; N, 8.98; Cl, 7.58.

### EXAMPLE 37

### AMD7087: Synthesis of the Ru(III) complex of phenylenediamine-N,N,N′,N′-tetraacetic acid (H₄pdta).

Phenylenediamine-N,N,N′,N′-tetraacetic acid tetramethyl ester

1,2-phenylenediamine (1.4 g, 1.3 mmol), methyl bromoacetate (12.3 mL, 13 mmol) and K₂CO₃ (17.9 g, 13 mmol) were heated at 85° C. in DMF (130 mL) under an inert atmosphere for 3 days. The DMF was removed under reduced pressure and the residue was dissolved in CH₂Cl₂. The solution was washed with an aqueous solution of saturated NH₄Cl and then H₂O. The organic layer was dried (MgSO₄) and evaporated to give a brown oil. This brown oil was triturated with MeOH to yield a white solid, which was removed by filtration and washed with methanol (0.3 g, 5.8%).

¹H NMR (CDCl₃) δ 3.65 (s, 12H), 4.30 (s, 8H), 6.92-7.04 (m, 4H). FAB (+ve) m/z 397 [M+H]⁺. Anal. Calcd. for C₁₈H₂₄N₂O₈: C, 54.54; H, 6.10; N, 7.07. Found: C, 54.57; H, 6.21; N, 7.19.

Phenylenediamine-N,N,N′,N′-tetraacetic acid (H₄pdta)

The tetramethyl ester (0.1 g, 0.25 mmol) was suspended in MeOH/H₂O (25 mL, 3/1) and cooled to 0° C. Lithium hydroxide monohydrate (0.106 g, 2.5 mmol) was added to the suspension and the reaction mixture was stirred in the dark overnight (during which time it was allowed to warm to room temperature). The clear solution was acidified with HCl (2N) and the solvent was removed under reduced pressure to leave a white solid.

¹H NMR (D₂O/K₂CO₃) δ 4.27 (s, 8H), 7.25-7.4 (m, 4H). The white solid was used without further purification to prepare the ruthenium complex.

Preparation of [Ru(Hpdta)(OH₂)].3H₂O

[Hydrogen aqua[N-bis((2-carboxy-κO)methyl)imino-κN]-1,2-phendiyl(2-(carboxy-κO)methyl)glycinato-κN]ruthenium (III)]

H₄pdta.xLiCl (0.25 mmol) was heated in HCl (3 mL, 1 mM) until completely dissolved. K₂[RuCl₅(OH₂)] (0.095 g, 0.25 mmol) was added to the ligand solution and the reaction mixture was heated to reflux for 1.5 hours. The solution was allowed to cool to room temperature and the yellow-green precipitate which formed was collected by filtration and washed with H₂O, EtOH and Et₂O (15 mg, 12%).

Anal. Calcd. for C₁₄H₁₅N₂O₉Ru.3H₂O: C, 32.95; H, 4.15; N, 5.49. Found: C, 32.65; H, 3.91; N, 5.58.

### EXAMPLE 38.

### AMD7459: Ruthenium (III) complex of N′-benzyldiethylenetriamine-N,N,N″,N″-tetraacetic acid (bdtta).

N-(hydroxyethyl)iminodiacetic acid di-t-butyl ester

Ethanolamine (1.84 g, 0.03 mol) was dissolved in dry THF (300 mL) and triethylamine (12.3 g, 0.12 mol) was added. To this stirring solution t-butylbromoacetate (23.5 g, 0.12 mol) was added and the reaction mixture was stirred for 16 hours. The solvent was removed in vacuo and the residue partitioned between Et₂O (100 mL) and H₂O (100 mL). The aqueous layer was extracted with Et₂O (3×100 mL), and the combined organic portions were dried over MgSO₄. The suspension was filtered and the solvent was removed in vacuo to afford the product (7.75 g, 89%) as a white solid.

¹H NMR (CDCl₃) δ 1.46 (6, 18H), 2.89 (t, 2H, J=6.0 Hz), 3.45 (s, 4H), 3.53 (t, 2H, J=6.0 Hz), 3.75 (bs, 1H). ¹³C NMR (CDCl₃) δ 28.15, 56.68, 57.11, 59.37, 81.48, 171.48. ES-MS m/z 290 [M+H]⁺.

N-[(Methanesulfonyl)ethyl]iminodiacetic acid di-t-butyl ester

N-(hydroxyethyl)iminodiacetic acid di-t-butyl ester (7.50 g, 0.03 mol) was dissolved in dry CH₂Cl₂ (250 mL) and triethylamine (14.8 g, 0.15 mol) was added. The solution was cooled in an ice bath and methanesulfonylchloride (3.55 g, 0.03 mol) was added dropwise with stirring. The reaction mixture was slowly warmed to room temperature and stirred for a further 16 hours. The reaction was then quenched with saturated NaHCO₃ (150 mL) and the aqueous layer was extracted with CH₂Cl₂ (2×150 mL). The combined organic extracts were dried (MgSO₄), filtered, and the solvent was removed in vacuo to afford the product (9.5 g, 99%) as an oil.

¹H NMR (CDCl₃) δ 1.46 (s, 18H), 3.08 (m, 5H), 3.48 (s, 4H), 4.34 (t, 2H, J=6.0 Hz).

N′-benzyldiethylenetriamine-N,N,N″,N″-tetraAcetic acid tetra-t-butyl ester

General Procedure A

N-[(Methanesulfonyl)ethyl]iminodiacetic acid di-t-butyl ester (4.86 g, 13 mmol) was dissolved in dry acetonitrile (50 mL) and benzylamine (0.47 g, 4.4 mmol) was added with stirring. K₂CO₃ (2.4 g, 0.45 mol) was added and the suspension was stirred for 16 hours at 45° C. The solvent was removed in vacuo and the residue partitioned between CHCl₃ (100 mL) and saturated NaHCO₃ (100 mL). The aqueous portion was extracted with CHCl₃ (3×75 mL), and the combined organic extracts were dried (MgSO₄), filtered and the solvent was removed in vacuo to afford the crude product as a brown oil. The product was purified by column chromatography on silica gel (2% MeOH, 1% NEt₃, CH₂Cl₂) to afford the product (1.35 g, 37%) as a colorless oil.

¹H NMR (CDCl₃) δ 1.43 (s, 36H), 2.59 (t, 4H, J=6.0 Hz), 2.82 (t, 4H, J=6.0 Hz), 3.40 (s, 8H), 7.24 (m, 5H). ¹³C NMR (CDCl₃) δ 28.19, 52.08, 52.86, 56.16, 59.17, 80.75, 126.78, 128.14, 128.85, 139.62, 170.74. ES-MS m/z 650 [M+H]⁺.

N′-benzyldiethylenetriamine-N,N,N″,N″-tetraacetic acid.xTFA (bdtta)

General Procedure B

N′-Benzyldiethylenetriamine-N,N,N″,N″-tetracetic acid tetra-t-butyl ester (1.0 g, 1.5 mmol) was dissolved in trifluoroacetic acid (14.8 g, 130 mmol) and the solution was left stirring for 16 hours. The solvent was removed in vacuo and the residue was lyophilized to afford the product (1.19 g, 100%) as a white solid:

¹H NMR (D₂O) δ 3.38 (t, 4H, J=6.0 Hz), 3.48 (t, 4H, J=6.0 Hz), 3.73 (s, 8H), 4.43 (s, 4H), 7.51 (bs, 5H). ¹³C NMR (D₂O) δ 50.22, 50.85, 55.43, 59.04, 129.50, 130.05, 130.90, 131.39, 172.64.

Preparation of [Ru(H₂bdtta)Cl].4.5H₂O

[Dihydrogen chloro[[N,N′-[[(phenyimethyl)κN]-2,1-ethanediyl]bis[N-(carboxymethyl)glycinato-κN,κO]]ruthenium (III)]

General Procedure C

N′-Benzyldiethylenetriamine-N,N,N″,N″-tetraacetic acid (bdtta) (0.256 g, 0.33 mmol) was dissolved in 1 mM HCl (5 mL). K₂[RuCl₅(H₂O)] (0.124 g, 0.33 mmol) was added and the reaction mixture was heated to 100° C. for 1.5 hours. The solution was then cooled and a yellow/green powder was collected. The powder was washed with the mother liquor, H₂O (2×10 mL), and Et₂O (3×5 niL) to afford the product (0.078 g, 24%) as a light yellow powder.

Anal. Calcd. for C₁₉H₂₅N₃O₈RuCl.4.5 H₂O: C, 35.60; H, 5.35; N, 6.56; Cl, 5.53. Found: C, 35.62; H, 5.22; N, 6.47; Cl, 5.33. IR (CsI) ν(cm⁻¹) 1736 (CO₂H); 1657 (CO2−).

### EXAMPLE 39

### AMD7460: Ruthenium (III) complex of N′-[2-pyridyl(methylene)]diethylenetriatnine-N,N,N″,N″-tetraacetic acid (pdtta).

Using General Procedure A

N-[(Methanesulfonyl)ethylliminodiacetic acid di-t-butyl ester (3.14 g, 8.5 mmol) was reacted with aminomethylpyridine (0.23 g, 2.0 mmol) and the crude reaction mixture was purified by silica gel chromatography (5% MeOH/CH₂Cl₂). The product fractions were combined and partitioned between Et₂O (30 mL) and NaOH (15 mL 0.1M). The aqueous layer was extracted with Et₂O (3×20 mL), and the combined organic extracts were dried (MgSO₄), filtered and the solvent removed in vacuo to afford the product (0.38 g, 30%) as an oil.

¹H NMR (CDCl₃) δ 1.40 (s, 36H), 2.64 (t, 4H, J=6.0 Hz), 2.81 (t, 4H, J=6.0 Hz), 3.38 (s, 8H), 3.76 (s, 2H), 7.08 (t, 11H, J=6.0 Hz), 7.45 (d, 11H, J=6.0 Hz), 7.57 (t, 1H, J=6.0 Hz), 8.46 (d, 1H, 6.0 Hz). ¹³C NMR (CDCl₃) δ 28.28, 52.17, 53.31, 56.14, 60.94, 121.74, 122.90, 136.32, 148.86, 160.25, 170.69. ES-MS m/z 651 [M+H]⁺.

N′-[2-pyridyl(methylene)]diethylenetriamine-N,N,N″,N″-tetraacetic acid.xHCl (pdtta)

Using General Procedure B

The oil from above (0.381 g, 0.59 mmol) was treated with TFA (7.4 g, 65 mmol). The crude material was purified on Dowex cation exchange resin (H⁺ form, 50 W-200 mesh) to afford the product (0.225 g, 44%) as a white solid.

¹H NMR (D₂O) δ 3.09 (t, 4H, J=6.6 Hz), 3.61 (t, 4H, J=6.6 Hz), 3.86 (s, 2H), 4.20 (s, 8H), 7.97 (t, 1H, J=6.9 Hz), 8.03 (d, 11H, J=8.1 Hz), 8.53, (t, 11H, J=8.1 Hz), 8.70 (d, 11H, J=6.9 Hz).

Preparation of[Ru(H₂pdtta)Cl].2H₂O

[Dihydrogen chloro[[N,N′-[[(2-pyridinylmethyl)imino-κN]di-2,1-ethanediyl]bis[N-(carboxymethyl)glycinato-κN,κO]]] ruthenium (III)]

Using General Procedure C

Pdtta (0.225 g, 0.27 mmol) was reacted with K₂[RuCl₅(H₂O)] (0.095 g, 0.25 mmol).

Anal. Calcd. for C₁₈H₂₄O₈N₄RuCl.2H₂O.1.0KCl.0.75HCl: C, 30.94; H, 4.15; N, 8.02; Cl, 13.95. Found: C, 30.85; H, 4.30; N, 8.01; Cl, 13.54. IR (CsI) ν(cm⁻¹) 1740 (CO₂H); 1657 (CO₂−); 311 (Ru—Cl).

### EXAMPLE 40

### AMD8676: Ruthenium (III) complex of N′-butyldiethylenetriamine-N,N,N″,N″-tetraacetic acid (budtta).

N′-butyldiethylenetriamine-N,N,N″,N″-tetraacetic acid tetra-t-butyl ester

Using General Procedure A

N-[(Methanesulfonyl)ethyl]iminodiacetic acid di-t-butyl ester (2.97 g, 8.1 mmol) was reacted with butylamine (0.20 g, 3.0 mmol) and the crude reaction mixture was purified by silica gel chromatography (5% MeOH/CH₂Cl₂) to afford the product (0.439 g, 27%) as a colorless oil.

¹H NMR (CDCl₃) δ 0.81 (t, 3H, J=6.0 Hz), 1.20 (m, 4H),1.38 (s, 36H), 2.38 (t, 2H, J=7.5 Hz), 2.54 (t, 4H, J=6.0 Hz), 2.71 (t, 4H, J=6.0 Hz), 3.37 (s, 8H). ¹³C NMR (CDCl₃) δ 14.36, 20.91, 28.49, 52.43, 53.61, 53.76, 54.92, 56.83, 81.31, 171.02. ES-MS m/z 616 [M+H]⁺.

N′-butyldiethylenetriamine-N,N,N″,N″-tetraacetic acid.xTFA (budtta)

Using General Procedure B

The oil from above (0.425 g, 0.69 mmol) was treated with TFA (14.8 g, 100 mmol) to afford the product (0.442 g, 87%) as an off-white solid.

¹H NMR (D₂O) δ 0.672 (bs, 3H), 0.81 (bs, 2H), 1.15 (bs, 2H), 2.71 (bs, 2H), 3.12 (bs, 8H), 3.56 (s, 8H). ES-MS m/z 448 [M+H]⁺.

Preparation of [Ru(H₂budtta)Cl].4H₂O

[Dihydrogen[[N,N′-[(butylimino-κN)di-2,1-ethanediyl]bis[N-(carboxymethyl)glycinato-κN,κO]]]chloro ruthenium (III)].

Using General Procedure C

Budtta (0.243 g, 0.33 mmol) was reacted with K₂[RuCl₅(H₂O)] (0.123 g, 0.33 mmol) to afford the product (0.083 g, 42%):

Anal. Calcd. for C₁₆H₂₇N₃O₈RuCl.4H₂O: C, 32.14; H, 5.90; N, 7.03; Cl, 5.93. Found: C, 32.23; H, 5.60; N, 6.94; Cl, 6.02. IR (CsI) ν(cm⁻¹) 1736 (CO₂H); 1657 (CO₂−); 411(Ru—Cl).

### EXAMPLE 41

### AMD8679: Ruthenium (III) complex of N′-ethyldiethylenetriamine-N,N,N″,N″-tetraacetic acid (edtta)

N′-ethyldiethylenetriamine-N,N,N″,N″-tetraacetic acid tetra-t-butyl ester

Using General Procedure A

N-[(Methanesulfonyl)ethyl]iminodiacetic acid di-t-butyl ester (3.169 g, 8.6 mmol) was reacted with ethylamine (0.13 g, 2.9 mmol) to afford, after purification by column chromatography on silica gel (2% MeOH, 1%NEt₃, CH₂Cl₂), the product (0.7 g, 55%) as a colorless oil.

¹H NMR (CDCl₃) δ 1.00 (t, 3H, J=6.0 Hz), 1.46 (s, 36H), 2.56 (m, 6H), 2.80 (t, 4H, J=7.5 Hz), 3.45 (s, 8H). ¹³C NMR (CDCl₃) δ 28.17, 48.16, 52.10, 52.61, 53.44, 56.30, 80.77, 170.70. ES-MS m/z 588 [M+H]⁺.

N′-ethyldiethylenetriamine-N,N,N″,N″-tetraacetic acid.xTFA (edtta)

Using General Procedure B

The oil from above (0.591 g, 1.01 mmol) was treated with TFA (14.8 g, 100 mmol) to afford the product (0.699 g, 98%) as an off-white solid.

¹H NMR (D₂O) δ 0.92 (t, 3H, J=6.9 Hz), 2.96 (d, 2H, J=6.9 Hz), 3.24 (s, 8H), 3.69 (s, 8H). ¹³C NMR (D₂O) δ 29.59, 49.19, 49.35, 49.95, 55.39, 170.68. ES-MS m/z 420 [M+H]⁺.

Preparation of [Ru(H₂edtta)Cl].H₂O [Dihydrogen chloro[[N,N′-[(ethylimino-κN)di-2,1-ethanediyl]bis[N-(carboxymethyl)glycinato-κN,κO]]] ruthenium (III)].

Using General Procedure C

Reaction of edtta (0.241 g, 0.34 mmol) with K₂[RuCl₅(H₂O)] (0.128 g, 0.34 mmol) afforded the product (0.0373 g, 21%). Anal. Calcd. for C₁₄H₂₃N₃O₈RuCl.1H₂O.0.1KCl: C, 32.13; H, 4.81; N, 8.03; Cl, 7.45. Found: C, 32.43; H, 4.80; N, 8.02; Cl, 7.81. IR (CsI) 1719 (CO₂H); 1678, 1601(CO₂); 415(Ru—Cl).

### EXAMPLE 42

### AMD8684: Ruthenium (III) complex of N′-phenyldiethylenetriamine-N,N,N″,N″-tetraacetic acid (phdtta)

N′-phenyldiethylenetriamine-N,N,N″N″-tetraacetic acid tetra-t-butyl ester

Using General Procedure A

Reaction of N-[(methanesulfonyl)ethyl]iminodiacetic acid di-t-butyl ester (3.358 g, 9.1 mmol) with aniline (0.28 g, 3.0 mmol) afforded, after purification by column chromatography on silica gel (4:1 Hexane: ethylacetate), the product (0.402 g, 21%) as a colorless oil.

¹H NMR (CDCl₃) δ 1.46 (s, 36H), 2.86 (t, 4H, J=7.5 Hz), 3.47 (bs, 12H), 6.62 (t, 1H, J=7.5 Hz), 6.70 (d, 1H, J=9.0), 7.17 (t, 1H, J=9.0 Hz).

N′-phenyldiethylenetriamine-N,N,N″,N″-tetraacetic acid.xTFA (phdtta)

Using General Procedure B

The oil from above (0.281 g, 0.44 mmol) was reacted with TFA (7.4 g, 50 mmol) affording the product (0.272 g, 81%) as an off-white solid.

¹H NMR (D₂O) δ 3.21 (m, 4H), 3.67 (t, 4H, J=6.6 Hz), 3.93 (s, 8H), 7.07 (t, 1H, J=7.8 Hz), 7.08 (t, 1H, J=7.8 Hz), 7.29 (t, 1H, J=7.5 Hz).

Preparation of [Ru(H₂phdtta)Cl].1.25H₂O

[Dihydrogen chlorof[N,N′-[(phenylimino-κN)di-2,1-ethanediyl]bis[N-(carboxymethyl)glycinato-κN,κO]]] ruthenium (III)].

Using General Procedure C

Reaction of phdtta (0.146 g, 0.18 mmol) with K₂[RuCl₅(H₂O)] (0.085 g, 0.23 mmol) afforded the product (0.0194 g, 16%).

Anal. Calcd. for C₁₈H₂₃N₃O₈RuCl.1.25H₂O.0.8KCl.0.8EtOH: C, 35.40; H, 4.59; N, 6.32; Cl, 9.60. Found: C, 35.73; H, 4.47; N, 5.93; Cl, 9.79. IR (CsI) ν(cm⁻¹) 1730 (CO₂H); 1611 (CO₂−); 403(Ru—Cl)

### EXAMPLE 43

### AMD7436: Ruthenium (III) complex of N,N″-bis-[2-pyridyl(methylene)]diethylenetriamine-N,N′,N″-triacetic acid (bpdtta)

N,N′,N″-Tritosyldiethylenetriamine

To a solution of tosyl chloride (21.18 g, 0.11 mol) in Et₂O (120 mL) was added diethylenetriamine (3.82 g, 0.04 mol). To this solution, an aqueous solution of NaOH (4.44 g, 0.11 mol) in de-ionized water (40 mL) was added dropwise. The resulting suspension was stirred for two hours and the white solid was collected by filtration and washed with H₂O and then Et₂O. The crude product was recrystallized from hot MeOH to afford the product (12.63 g, 60.4%) as a white crystalline solid.

¹H NMR (CDCl₃) δ 2.43 (bs, 9H), 3.06 (dt, 4H, J=5.5, 6.9 Hz), 3.17 (t, 4H, J=6.9 Hz), 6.55 (t, 2H, J=5.5 Hz), 7.40 (m, 6H), 7.63 (d, 2H, J=8.1 Hz), 7.74 (d, 4H, J=8.1 Hz). ¹³C NMR (acetone-d₆) δ 21.79, 43.51, 50.60, 128.26, 128.50, 130.92, 131.07, 137.27, 139.25, 144.38, 144.95. ES-MS m/z 588 [M+H]⁺.

2-[Methanesulfonyl(methyl)]pyridine

2-Pyridinemethanol (3.39 g, 31.1 mmol) and triethylamine (9.44 g, 93 mmol) were dissolved in dry CH₂Cl₂ (250 mL) and the resulting solution was cooled to 0° C. in an ice bath. Methanesulfonylchloride (4.27 g, 37.3 mmol) was added dropwise and the reaction mixture was stirred for 50 minutes. The reaction was then quenched with saturated NaHCO₃ (115 mL). The aqueous layer was washed with CH₂Cl₂ (2×50 mL), and the organic portions were combined and dried over MgSO₄. After filtering, the solvent was removed in vacuo to afford the product (6.5 g, 100%) as a red oil.

¹H NMR (CDCl₃) δ 3.11 (s, 3H), 5.33 (s, 2H), 7.30 (m, 1H), 7.48 (d, 1H, J=7.8 Hz), 7.77 (dd, 1H, J=1.7, 7.7 Hz), 8.59 (m, 1H).

N,N″-bis-[2-pyridyl(methylene)]-N,N′,N″-tritosyldiethylenetriamine

To a solution of N,N′,N″-tritosyldiethylenetriamine (8.8 g, 15.6 mmol) in DMF (75 mL) under a nitrogen atmosphere was added NaH (60% in oil, 1.24 g, 31.1 mmol) and the mixture was stirred for 45 minutes. 2-[Methanesulfonyl(methyl)]pyridine (6.5 g, 34.7 mmol) dissolved in 10 mL CH₂Cl₂ was then added and the reaction was heated to 80° C. for 20 hours. Ethanol was then added and the DMF was removed in vacuo. The residue was dissolved in CH₂Cl₂ and washed with brine (3×100 mL), saturated NH₄Cl solution (3×100 mL), and finally a saturated aqueous solution of K₂CO₃ (3×100 mL). The organic layer was dried over Na₂SO₄, filtered and the solvent was removed in vacuo to afford the crude product (9.0 g) as an off-white solid.

¹H NMR δ 2.42 (bs, 12H), 3.04 (m, 4H), 3.30 (m, 4H), 4.41 (s, 4H), 7.39 (m, 10H), 7.71 (m, 8H), 8.48 (m, 2H). ES-MS m/z 748 [M+H]⁺. This product was used without further purification.

N,N″-bis-[2-pyridyl(methylene)]diethylenetriamine

The solid from above (3.79 g, 5.1 mmol) was added to 13 mL concentrated H₂SO₄ maintained at a temperature of 120° C. After 5 minutes the reaction mixture was cooled and EtOH (90 mL) was added resulting in the precipitation of a brown solid. The solid was collected by filtration, dissolved in H₂O (100 mL) and heated in the presence of activated charcoal. The mixture was filtered through celite and the volume of the filtrate was reduced to approximately 20 mL and then concentrated HCl (20 mL) was added. Most of the solvent was removed in vacuo and cold EtOH was added to precipitate a white solid. The white solid was then dissolved in H₂O and the pH was adjusted to 12 with 3M NaOH. The aqueous solution was extracted with CHCl₃ (3×50 mL), and the combined organic extracts were dried (MgSO₄). Evaporation of the solvent afforded the product (0.785 g, 54%) as a colorless oil.

¹H NMR δ 2.43 (s, 3H), 2.80 (s, 8H), 3.92 (s, 4H), 7.14 (t, 2H, J=6.0 Hz), 7.30 (d, 2H, J=6.0 Hz), 7.62 (dd, 2H, J=3.0, 6.0 Hz), 8.53 (d, 2H, J=3.0 Hz).

N,N″-bis-[2-pyridyl(methylene)]diethylenetriamine-N,N′,N″-triacetic acid tri-t-butyl ester

The oil from above (0.737 g, 2.59 mmol) was dissolved in dry toluene (20 mL), containing t-butylbromoacetate (3.02 g, 15.50 mmol) and triethylamine (5.20 g, 51.0 mmol) and the reaction mixture was stirred overnight. After 16 hours the solvent was removed in vacuo and the residue was partitioned between Et₂O (40 mL) and H₂O (40 mL). The aqueous portion was extracted with Et₂O (2×40 mL) and the organic portions were combined, and dried over MgSO₄. Removal of the solvent in vacuo afforded the desired product (1.00 g, 62%) as an oil.

¹H NMR (CDCl₃) δ 1.40 (s, 9H), 1.45 (s, 18H), 2.75 (s, 8H), 3.27 (s, 2H), 3.32 (s, 4H), 3.91 (s, 4H), 7.12 (t, 2H, 6.0 Hz), 7.50 (d, 2H, 6.0 Hz), 7.62 (dd, 2H, J=3.0, 6.0 Hz), 8.50 (d, 2H, J=3 Hz). ES-MS m/z 628 [M+H]⁺.

N,N″-bis[2-pyridyl(methylene)]diethylenetriamine-N,N′,N″-triaceticacid.5TFA (bpdtta)

The oil from above (1.45 g, 2.30 mmol) was dissolved in trifluoroacetic acid (8.8 g, 78 mmol) and left stirring for 16 hours. The solvent was removed in vacuo and the resulting oil was lyophilized. An off-white powder was obtained (2.05 g, 86%).

¹H NMR (acetone-d₆) δ 3.50 (t, 4H, J=5.7 Hz), 3.69 (s, 4H), 3.79 (t, 4H, J=5.7 Hz), 4.41 (s, 2H), 4.53 (s, 411), 8.04 (t, 2H, J=6.4 Hz), 8.13 (d, 2H, J=6.4 Hz), 8.59 (t, 2H, J=7.9 Hz), 8.92 (d, 2H, J=7.9 Hz). ES-MS m/z 461 [M+H]⁺. Anal. Calcd. for C₂₂H₂₉N₅O₆.5TFA.2.5H₂O: C, 35.77; H, 3.66; N, 6.34. Found: C, 35.54; H, 3.30; N, 6.18.

Preparation of [Ru(H₂bpdtta)][CF₃CO₂]2.3H₂O

[N-[2-[[(carboxy-κO)methyl][(2-pyridinyl-κN)methyl]amino-κN]ethyl-N-[2-[(carboxymethyl)[(2-pyridinyl-κN]methyl]amino-κN]ethyl]glycinato-κN] ruthenium (III) bis(trifluoroacetate).

Bpdtta (0.37g, 0.35 mmol) was dissolved in 1 mM HCl (3 mL) and the pH was adjusted to 4 with 1M NaOH. K₂[RuCl,(H₂)] (0.13 g, 0.35 mmol), dissolved in a minimum amount of 1 mM HCl was added to the reaction mixture. The solution was refluxed for 1.5 hours and then cooled in an ice bath. The residue was passed through Sephadex gel (G-10) and a yellow band was collected and lyophilized (0.11 g, 37%).

Anal. Calcd. for C₂₂H₂₈N₅O₆Ru.2TFA.3H₂O: C, 37.19; H, 4.08; N, 8.34. Found: C, 37.16; H, 4.00; N. 8.62. IR (CsI) ν(cm⁻¹) 1688 (Co₂H); 1630(CO₂−).

### EXAMPLE 44

### AMD8701: Ruthenium (III) complex of 1,3-Propanediamine-N,N,N′,N′-tetraacetic acid (pdta).

1,3-Propanediamine-N,N,N′,N′tetraacetic acid tetra-t-butyl ester

1,3-propanediamine (0.528 g, 7.1 mmol) was dissolved in a mixture of dry THF (50 mL), triethylamine (5.76 g, 57 mmol) and t-butylbromoacetate (8.34 g, 43 mmol) and the reaction mixture was stirred under a nitrogen atmosphere for 24 hours. The solvent was then removed in vacuo and the residue partitioned between CHCl₃ (40 mL) and saturated NaHCO₃ (30 mL). The aqueous portion was extracted with CHCl₃ (3×30 mL), and the combined organic portions were dried over MgSO₄, filtered, and the solvent removed in vacuo. The crude material was purified by silica gel chromatography (4:1 Hexanes: EtOAc) afforded the product (3.00 g, 80%) as a colorless oil.

¹H NMR (CDCl₃) δ 1.45 (s, 36H), 1.63-1.68 (m, 2H), 2.73 (dd, 4H, J=6.0, 9.0 Hz), 3.42 (s, 8H). ¹³C NMR δ 28.18, 51.93, 55.76, 80.80, 170.74. ES-MS m/z 531 [M+H]⁺.

1,3-Propanediamine-N,N,N′,N′-tetraacetic acid.xTFA (pdta)

Using General Procedure B

Reaction of the oil from above (0.866 g, 1.63 mmol) with TFA (8.88 g, 78 mmol) afforded the product (0.8405 g, 96%).

¹H NMR (CD₃OD) δ 2.15-2.19 (m, 2H), 3.43 (t, 4H, J=6.0 Hz), 4.16 (s, 8H). ES-MS m/z 307 [M+H]⁺.

Preparation of K[Ru(H₂pdta)Cl₂].3H₂O

[Potassium dihydrogen dichloro[[N,N′-1,3-propanediylbis[N-(carboxymethyl)glycinato-κN,κO]]] ruthenium (III)]

Using General Procedure C

Reaction of pdta (0.291 g, 0.54 mmol) with K₂[RuCl₅(H₂O)] (0.203 g, 0.54 mmol) afforded the product (0.075 g, 24%) as a yellow solid.

Anal. Calcd. for C₁₁H₁₆N₂O₈Cl₂RuK.3.0H₂O: C, 23.20; H, 3.89; N, 4.92; Cl, 12.45. Found: C, 22.97; H, 3.67; N, 4.80; Cl, 12.15. IR (CsI) ν(cm⁻¹) 1738 (CO₂H); 1642 (CO₂−); 316(Ru—Cl).

### EXAMPLE 45

### AMD7494: Ruthenium (III) complex of N-[2-(carboxy)-6-pyridyl(methylene)]iminodiacetic acid (cpida).

Methyl 2-(hydroxymethyl)pyridinecarboxylate

Dimethyl-2,6-pyridinedicarboxylate (1.057 g, 5.4 mmol) was dissolved in dry CH₂Cl₂ (45 mL) and the solution was cooled to −78° C. DIBAL-H (11 mL, 10.8 mmol) was added dropwise with stirring and the solution was stirred at −78° C. for 0.5 hours and then slowly warmed to room temperature over a period of 1 hour. The reaction was quenched with H₂O (15 mL) /sodium potassium tartrate (15 mL) and extracted with CH₂Cl₂ (3×80 mL). The combined organic extracts were dried (MgSO₄) and evaporated in vacuo to afford the crude product. Purification by column chromatography on silica gel (4:1 Hexanes: Ethyl acetate to 10% MeOH/CH₂Cl₂) afforded the desired product (0.220 g, 26%) as a colorless oil.

¹H NMR (CDCl₃) δ 3.33 (t, 1H, J=4.5 Hz), 4.00 (s, 3H), 4.87 (d, 2H, J=4.5 Hz), 7.54 (d, 1H, J=6.0), 7.83 (dd, 1H, J=6.0, 9.0), 8.00 (d, 1H, J=9.0 Hz).

Methyl 2-(methanesulfonylmethyl)pyridinecarboxylate

To a stirred solution of methyl 2-(hydroxymethyl)pyridinecarboxylate (0.220 g, 1.3 mmol) dissolved in dry CH₂Cl₂ (13 mL) and triethylamine (0.40 g, 4.0 mmol) cooled in an ice bath was added dropwise, methanesulfonylchloride (0.18 g, 1.6 mmol). After 30 minutes the reaction was quenched with saturated NaHCO₃ (15 mL) and the aqueous phase was separated and extracted with CH₂Cl₂ (3×15 mL). The combined organic extracts were dried (MgSO₄) and the solvent was evaporated in vacuo to afford the product (0.347 g, 100%) as a yellow orange oil.

¹H NMR (CDCl₃) δ 3.15 (s, 3H), 4.01 (s, 3H), 5.44 (s, 2H), 7.70 (d, 1H, J=6.0 Hz), 7.92 (dd, 1H, J=6.0, 9.0 Hz), 8.12 (d, 1H, J=9.0 Hz).

N-[2-(carboxymethyl)-6-pyridyl(methylene)]iminodiacetic acid dimethyl ester

General Procedure D

The oil from above (0.323 g, 1.3 mmol) was dissolved in dry DMF (13 mL) and iminodiacetic acid dimethyl ester (0.191 g, 1.2 mmol) was added. Once the reagents had dissolved, K₂CO₃ (0.36 g, 2.6 mmol) was added and the reaction mixture was stirred at 35° C. for 16 hours. The solvent was removed in vacuo and partitioned between H₂O (10 mL) and CH₂Cl₂ (15 mL). The aqueous portion was extracted with CH₂Cl₂ (3×15 mL), and the combined organic extracts were dried (MgSO₄) and evaporated in vacuo. The crude material was purified by silica gel chromatography (75% EtOAc/hexanes) to afford the product (0.200 g, 49%) as a colorless oil.

¹H NMR (CDCl₃) δ 3.70 (s, 6H), 3.97 (s, 311), 4.16 (s, 4H), 5.36 (s, 2H), 7.51 (d, 1H, J=9.0), 7.84 (dd, 1H, J=6.0, 9.0), 8.02 (d, 1H, J=6.0 Hz). ¹³C NMR δ 49.48, 52.63, 53.32, 68.46, 124.46, 124.79, 138.25, 155.93, 157.31, 165.88, 170.09.

N-[2-(carboxy)-6-pyridyl(methylene)]iminodiacetic acid.xHCl (cpida)

The oil from above (0.200 g, 0.65 mmol) was dissolved in MeOH (19 mL) and H₂O (6 mL) and the solution was cooled to 0° C. using an ice bath. Lithium hydroxide monohydrate (0.270 g, 6.4 mmol) was added and the mixture was stirred for 17 hours at room temperature in the absence of light. The solution was acidified with 2N HCl and the solvent was removed in vacuo. The crude material was purified on Dowex cation exchange resin (H⁺form, 50 W-200 mesh) to afford the product (0.172 g, 78%).

¹H NMR (D₂O) δ 4.02 (s, 2H), 4.15 (s, 2H), 5.39 (s, 2H), 7.95 (d, 11H, J=7.5 Hz), 8.25 (d, 11H, J=7.2 Hz), 8.46 (dd, 1H, J=7.2, 7.5 Hz). ¹³C NMR (D₂O) δ 50.27, 50.56, 127.02, 128.74, 147.29, 152.83, 156.73, 173.22, 173.46. ES-MS m/z 313 [M+H]⁺.

Preparation of [Ru(Hcpida)(OH₂)(Cl)].1.5H₂O

[Hydrogen aqua[6-[[[(carboxy-κO)methyl](carboxymethyl)amino-κN]methyl]-2-pyridinecarboxylato-κN¹,κO²]chloro ruthenium (III)].

Using General Procedure C

Reaction of cpida (0.157 g, 0.48 mmol) with K₂[RuCl₅(H₂O)] (0.172 g, 0.46 mmol) afforded the product.

Anal. Calcd. for C₁₁H₁₂N₂O₇RuCl.1.5H₂O.0.9KCl: C, 25.66; H, 2.94; N, 5.44; Cl, 13.08. Found: C, 25.56; H, 2.64; N, 5.06; Cl, 12.97. IR (CsI): ν(cm⁻¹) 1709 (CO2H); 1632, 607(CO₂−); 341(Ru—Cl).

### EXAMPLE 46

### AMD7493: Ruthenium (III) complex of N-[2-(Hydroxymethyl)-6-pyridyl(methylene)]iminodiacetic acid (hpida).

2-[Methanesulfonyl(methylene)]-6-pyridinecarboxaldehyde

2-(Hydroxymethyl)-6-pyridinecarboxaldehyde (2.30 g, 0.017 mol) was dissolved in dry CH₂Cl₂ (160 mL) containing triethylamine (5.08 g, 0.05 mol). The solution was cooled to 0° C. in an ice bath and methanesulfonylchloride (2.12 g, 0.018 mol) was added dropwise. Stirring was continued for 0.5 hours and the reaction was quenched with saturated NaHCO₃ (160 mL). The aqueous portion was extracted with CH₂Cl₂ (3×150 mL), and the combined organic extracts were dried (Na₂SO₄) and the solvent was removed in vacuo to afford the product (3.61 g, 100%) as a brown oil.

¹H NMR (CDCl₃) δ 3.15 (s, 3H), 5.43 (s, 2H), 7.70 (m, 1H), 7.97 (m, 2H), 10.05 (s, 1H). This was used without further purification.

Using General Procedure D

Reaction of the oil from above (3.61 g, 0.017 mol) with iminodiaceticacid di-t-butyl ester (3.706 g, 0.015 mmol) afforded, after column chromatography on silica (4:1 hexanes: EtOAc), the product (2.136 g, 40%) as a colorless oil.

¹H NMR (CDCl₃) δ 1.46 (s, 18H), 3.50 (s, 4H), 4.14 (s, 2H), 7.85 (m, 1H), 7.94 (m, 1H), 10.05 (s, 1H).

N-[2-(Hydroxymethyl)-6-pyridyl(methylene)]iminodiacetic acid di-t-butyl ester

The oil from above (2.25 g, 6.2 mmol) was dissolved in dry MeOH (60 mL) under a nitrogen atmosphere. Sodium borohydride (0.235 g, 6.2 mmol) was added in one portion and the reaction was heated to 60° C. with stirring. After 1 hour the solvent was removed in vacuo and the residue was partitioned between H₂O (30 mL) and CH₂Cl₂ (30 mL). The aqueous phase was separated and extracted with CH₂Cl₂ (3×40 mL) and the combined organic extracts were dried (MgSO₄) and evaporated in vacuo to afford the product (2.16 g, 95%) as a colorless oil.

¹H NMR (CDCl₃) δ 1.46 (s, 18H), 3.48 (s, 4H), 3.98 (t, 1H, J=4.5 Hz), 4.05 (s, 2H), 4.72 (d, 2H, J=4.5 Hz), 7.08 (d, 1H, J=6.0 Hz), 7.53 (d, 1H, J=9.0 Hz), 7.66 (dd, 1H, J=6.0, 9.0 Hz). ¹³C NMR (CDCl₃) δ 28.57, 56.22, 59.88, 64.13, 81.47, 119.04, 122.02, 137.64, 158.25, 158.65, 170.90. ES-MS m/z 367 [M+H]⁺.

N-[2-(Hydroxymethyl)-6-pyridyl(methylene)]iminodiacetic acid.xTFA (hpida)

Using General Procedure B

Reaction of N-[2-(Hydroxymethyl)-6-pyridyl(methylene)]iminodiacetic acid di-t-butyl ester with TFA (4.44 g, 40 mmol) afforded the product (0.492 g, 100%) as a white solid.

¹H NMR (D₂O) δ 3.64 (s, 4H), 4.28 (s, 2H), 4.85 (s, 2H), 7.69 (bs, 2H), 8.27 (t, 1H, J=8.0 Hz). ¹³C NMR (D₂O) δ 55.98, 60.07, 123.75, 125.19, 147.02, 152.72, 155.65, 174.85. ES-MS m/z 255 [M+H]⁺.

Preparation of [Ru(Hhpida)(OH₂)Cl₂].H₂O

[Hydrogen aqua[N-(carboxymethyl)-N-[[6-(hydroxymethyl)-2-pyridinyl-κN]methyl]glycinato-κN,κO]dichloro ruthenium (III)].

Following General Procedure C

Reaction of hpida (0.152 g, 0.32 mmol) with K₂RuCl₅(H₂O)] (0.118 g, 0.32 mmol) afforded the product (0.0352 g, 24%).

Anal. Calcd. for C₁₁H₁₅N₂O₆Cl₂Ru.H₂O: C, 28.64; H, 3.71; N, 6.07; Cl, 15.37. Found: C, 28.44; H, 3.67; N, 6.02; Cl, 15.36. IR (CsI) ν(cm⁻¹) 1657, 1630(CO₂−); 316(Ru—Cl).

### EXAMPLE 47

### AMD8699: Ruthenium (III) complex of N-[2-(benzyloxymethyl)-6-pyridyl(methylene)]iminodiacetic acid (bpida)

2-(Benzyloxymethyl)-6-(hydroxymethyl)pyridine

2,6-Pyridinedimethanol (1.523 g, 0.011 mol) was dissolved in DMSO (5 mL) and powdered KOH (0.63 g, 0.011 mol) was added. After 10 minutes benzylbromide (1.87 g, 0.011 mol) was added and the reaction was heated to 80° C. for 17 hours. The reaction mixture was quenched with H₂O (9 mL) and extracted with Et₂O (3×25 mL). The combined organic extracts were dried (MgSO₄) and the solvent was evaporated in vacuo. The crude product was purified by column chromatography on silica (1:1 hexanes: EtOAc and then EtOAc) to afford the product (0.971 g, 39%) as a colorless oil.

¹H NMR (CDCl₃) δ 3.79 (bs, 1H), 4.66 (s, 211), 4.70 (s, 2H), 7.48 (d, 2H, J=3.6 Hz), 7.13 (d, 1H, J=7.5 Hz), 7.32-7.43 (m, 6H), 7.70 (dd, 1H, J=7.2, 7.8 Hz). ¹³C NMR (CDCl₃) δ 60.40, 63.89, 72.96, 119.01, 119.91, 127.80, 128.48, 137.31, 137.94, 157.57, 158.16.

2-(Benzyloxymethyl)-6-(methanesulfonylmethyl)pyridine

The oil from above (0.971 g, 4.24 mmol) was dissolved in dry CH₂Cl₂ (40 mL) containing triethylamine (1.29 g, 12.7 mmol) under a nitrogen atmosphere and the solution was cooled to 0° C. with stirring in an ice bath. Methanesulfonylchloride (0.577 g, 5.0 mmol) was then added dropwise and the mixture was stirred for 45 minutes and then quenched with saturated NaHCO₃ (30 mL). The separated aqueous phase was extracted with CH₂Cl₂ (2×20 mL) and the combined organic extracts were dried (MgSO₄) and evaporated in vacuo to afford the product (1.18 g, 91%) as a brown oil.

¹H NMR (CDCl₃) δ 3.07 (s, 3H), 4.65 (s, 2H), 4.67 (s, 2H), 5.29 (s, 2H), 7.27-7.38 (m, 6H), 7.50 (d, 1H, J=9.0 Hz), 7.77 (dd, 1H, J=6.0, 9.0 Hz).

N-[2-(benzyloxymethyl)-6-pyridyl(methylene)liminodiaceticacid di-t-butyl ester

Using General Procedure D

Reaction of the oil from above (1.18 g, 3.84 mmol) with iminodiacetic acid di-t-butyl ester (0.85 g, 3.47 mmol) afforded, after silica gel chromatography (4:1 Hexanes: EtOAc), the product (0.772 g, 45%) as a colorless oil.

¹H NMR (CDCl₃) δ 1.45 (s, 18H), 3.48 (s, 411), 4.03 (s, 2H), 4.65 (s, 211), 4.67 (s, 2H), 7.27-7.38 (m, 6H), 7.54 (d, 1H, J=7.5 Hz), 7.68 (dd, 1H, J=7.5, 7.8 Hz). ¹³C NMR (CDCl₃) δ 28.19, 55.78, 59.83, 72.92, 73.26, 80.98, 119.58, 121.46, 127.71, 127.83, 128.42, 137.16, 138.09, 157.82, 158.86, 170.53. ES-MS m/z 457 [M+H]⁺.

N-[2-(benzyloxymethyl)-6-pyridyl(methylene)]iminodiacetic acid.xTFA (bpida).

Using General Procedure B

Reaction of the product from above (0.7 g, 1.53 mmol) with TFA (10.36 g, 90 mmol) afforded the product (0.876 g, 100%) as a yellow viscous oil.

¹H NMR (D₂O) δ 3.77 (s, 4H), 4.44 (s, 2H), 4.75 (s, 2H), 4.92 (s, 2H), 7.33-7.41 (m, 5H), 7.76 (d, 1H, J=9.0 Hz), 7.83 (d, 1H, J=6.0 Hz), 8.33 (dd, 1H, J=6.0, 9.0 Hz). ¹³C NMR (D₂O) δ 55.73, 56.51, 67.68, 68.27, 73.62, 123.45, 124.33, 128.18, 128.58, 137.52, 144.88, 154.30, 172.94. ES-MS m/z 345 [M+H]⁺.

Preparation of[Ru(bpida)Cl(OH₂)]

[Aqua[N-[(carboxy-κO)methyl]-N-[[6-[(phenylmethoxy)methyl]-2-pyridinyl-κN methyl]glycinato-κN,κO]chloro ruthenium (III)]

Using General Procedure C

Reaction of bpida (0.376 g, 0.66 mmol) with K₂[RuCl₅(H₂O)] (0.247 g, 0.66 mmol) afforded the product (0.0910 g, 26%) as a yellow solid.

Anal. Calcd. for C₁₈H₂₀N₂O₆RuCl.0.4KCl: C, 41.05; H, 3.83; N, 5.32; Cl, 9.42. Found: C, 41.30; H, 3.95; N, 5.27; Cl, 9.83. IR (CsI) ν(cm⁻¹) 1657(CO₂−); 391(Ru—Cl).

### EXAMPLE 48

### AMD8677: Ruthenium (III) complex of N-[(3-carboxymethyl)benzyl]ethylenediamine-N,N′,N′-triacetic acid (cmbedta)

Ethylenediamine-N,N′,N′-triacetic acid tri-t-butyl ester

To a stirred solution of ethylenediamine (0.50 g, 8.3 mmol) in dry THF (70 mL) and triethylamine (3.34 g, 33 mmol) was added t-butylbromoacetate (4.9 g, 25 mmol) and the reaction mixture was stirred for 16 hours at room temperature. The solvent was removed in vacuo and the residue was partitioned between CH₂Cl₂ (80 mL) and H₂O (50 mL). The separated aqueous phase was extracted with CH₂Cl₂ (2×80 mL) and the combined organic extracts were dried (MgSO₄) and evaporated in vacuo. The crude material was purified by column chromatography on silica gel (5% MeOH/CH₂Cl₂) to afford the product (0.887 g, 27%) as an oil.

¹H NMR (CDCl₃) δ 1.43 (s, 27H), 2.63 (t, 2H, J=6.0 Hz), 2.84 (t, 2H, J=6.0 Hz), 3.28 (s, 2H), 3.42 (s, 4H). ¹³C NMR (CDCl₃) δ 28.46, 28.51, 47.42, 51.84, 54.15, 56.41, 81.31, 81.36, 171.22, 171.68.

N-[(3-carboxymethyl)benzyllethylenediamine-N,N′,N′-triacetic acid tri-t-butyl ester

General Procedure E

To a stirred solution of the oil from above (0.165 g, 0.41 mmol) in dry THF (5 mL) and triethylamine (0.087 g, 0.86 mmol) was added 3-bromomethylbenzoate (0.094 g, 0.41 mmol) and the reaction was stirred at 35° C. for 22 hours. The solvent was removed in vacuo and the residue was partitioned between CH₂Cl₂ (10 mL) and saturated NaHCO₃ (10 mL). The separated aqueous phase was extracted with CH₂Cl₂ (2×10 mL), and the combined organic extracts were dried (MgSO₄) and evaporated in vacuo. The crude material was purified by radial chromatography on silica gel (7:1 Hexanes:EtOAc) to afford the product (0.115 g, 51%) as a colorless oil.

¹H NMR (CDCl₃) δ 1.40 (s, 18H), 1.43 (s, 9H), 2.79-2.86 (m, 4H), 3.25 (s, 2H), 3.40 (s, 4H), 3.83 (s, 2H), 3.87 (s, 3H), 7.35 (dd, 1H, J=6.0, 9.0 Hz), 7.55 (d, 1H, J=9.0 Hz), 7.89 (d, 1H, J=6.0 Hz), 7.95 (s, 1H).

N-[(3-carboxymethyl)benzyllethylenediamine-N,N′,N′-triacetic acid.xTFA (cmbedta)

Using General Procedure B

Reaction of the oil from above (0.115 g, 0.21 mmol) with TFA (7.4 g, 65 mmol) afforded the product (0.094 g, 74%) as a light brown solid.

¹H NMR (D₂O) δ 3.16 (bs, 2H), 3.43-3.48 (m, 6H), 3.90 (s, 3H), 4.09 (s, 2H), 4.63 (s, 2H), 7.58 (t, 1H, J=7.8 Hz), 7.83 (d, 1H, J=7.8 Hz), 8.10 (d, 1H, J=7.8 Hz), 8.23 (s, 11H). ¹³C NMR (D₂O) 650.93, 53.38, 54.09, 54.53, 56.27, 60.46, 131.15, 132.48, 132.59, 132.78, 133.58, 137.21, 168.28, 169.47, 175.47.

Preparation of K[Ru(cmbedta)CI].H₂O

[Potassium chloro[methyl 3-[[[2-[bis[(carboxy-κO)methyl]amino-κN]ethyl][(carboxy-κO)methyl]amino-κN]methyl]benzoato ruthenium (III)]

Using General Procedure C

Reaction of cmbedta (0.094 g, 0.16 mmol) with K₂[RuCl₅(H₂O)] (0.058 g, 0.16 mmol) afforded the product (0.0334 g, 36%) as a yellow solid.

Anal. Calcd. for C₁₇H₁₉N₂O₈RuClK.0.15KCl.H₂O: C, 34.95; H, 3.62; N, 4.80; Cl, 6.98. Found: C, 35.19; H, 3.92; N, 4.80; Cl, 7.28. IR (CsI) ν(cm⁻¹) 1728 (CO₂Me); 1686(CO₂−); 386(Ru—Cl).

### EXAMPLE 49

### AMD8893: Ruthenium (III) Complex of N-[2-(N-acetylpyrrolidine)]ethylenediamine-N,N′,N′-triacetic acid (apedta).

Chloroacetylpyrrolidine

A solution of chloroacetyl chloride (3.6 mL, 45.0 mmol) in anhydrous THF (10 mL) was added dropwise to a stirred mixture of pyrrolidine (2.56 g, 36.0 mmol) and potassium carbonate (7.46 g, 54.0 mmol) in anhydrous THF (50 mL) cooled to 0° C. The reaction mixture was stirred at 0° C. for 30 minutes and the reaction mixture was then evaporated to give a white solid. The solid was partitioned between CH₂Cl₂ and H₂O and the aqueous layer was extracted twice with CH₂Cl₂. The combined organic phases were washed twice with H₂O, twice with NH₄Cl (1 N) then dried (MgSO₄) and evaporated to give a yellow oil (2.97 g, 55.9%).

¹H NMR (CDCl₃) δ 1.84 (m, 2H), 2.02 (m, 2H), 3.52 (q, 4H, J=6.0 Hz), 4.02 (s, 2H).

N-[2-(N-acetylpyrrolidine)]ethylenediamine-N,N′,N′-triacetic acid tri-t-butyl ester

Potassium carbonate (0.69 g, 4.98 mmol) was added to a solution of ethylenediamine-N,N′,N′-triacetic acid tri-t-butyl ester (0.80 g, 1.99 mmol) and chloroacetylpyrrolidine (0.59 g, 3.98 mmol) in anhydrous acetonitrile (20 mL). The mixture was heated to reflux for 60 hours under N₂ and then evaporated. The orange residue was dissolved in a mixture of CH₂Cl₂ and K₂CO₃ (saturated). The aqueous layer was then separated and extracted twice with CH₂Cl₂. The combined organic phases were washed twice with saturated aqueous K₂CO₃, dried (MgSO₄) and evaporated. The resulting orange oil was purified twice on silica gel using centrifugal chromatography (using CH₂Cl₂ as the eluent) to afford the desired compound as a yellow oil (0.48 g, 47%).

¹H NMR (CDCl₃) δ 1.44 (s, 27H), 1.86 (m, 2H), 1.94 (m, 2H), 2.87 (s, 4H), 3.45 (s, br, 6H), 3.50 (s, 4H), 3.55 (s, 2H). ES-MS m/z 514 [M+H]⁺.

N-[2-(N-acetylpyrrolidine)]ethylenediamine-N,N′,N′-triacetic acid.xTFA (apedta)

Trifluoroacetic acid (1.0 mL, 0.49 mmol) was added to a solution of the product from above (0.25 g, 12.98 mmol) in anhydrous CH₂Cl₂ (5 mL) and the mixture was stirred overnight at room temperature under nitrogen. The reaction mixture was evaporated and then lyophilized to afford the desired compound as a pale yellow solid (0.21 g, 74.7%).

¹H NMR (D₂O) δ 1.88 (m, 4H), 3.38 (m, 6H), 3.53 (t, 2H, J=4.8 Hz), 3.82 (s, 4H), 4.15 (s, 2H), 4.27 (s, 2H). ¹³C NMR (D₂O) 6 24.03, 25.66, 46.41, 46.94, 50.28, 53.32, 55.32, 56.00, 56.46, 164.36, 169.51, 172.94. ES-MS m/z 346[M+H]⁺, 368[M+Na]⁺, 384[M+K]⁺.

Preparation of[Ru(apedta)(OH₂)].1.2H₂O

[Aqua[N-[2-[bis[(carboxy-κO)methyl]amino-κN]ethyl]-N-[2-oxo-2-(1-pyrrolidinyl)ethyl]glycinato-κN,κO] ruthenium (III)].

Apedta (0.37 g, 0.65 mmol) was heated in HCl (1 mM, 6 mL) until completely dissolved. The pH of the solution was then adjusted to pH3.0 with KOH (1 N). K₂[RuCl₋₅(OH₂)] (0.24 g, 0.65 mmol) was added to the solution and the reaction mixture was heated to 100° C. for 2 hours. The solution was evaporated and purified by size exclusion column chromatography on Sephadex G-10 resin (H₂O) and the resulting solid was dried overnight in vacuo at 40° C. to afford a brown crystalline solid (0.062 g, 18.1%). ES-MS m/z 467[M—OH₂+Na]⁺. IR (CsI) ν(cm⁻¹) 1646 (C═O).

Anal. Calcd. for C₁₄H₂₂N₃O₈₋Ru.1.2 H₂O.0.6 KCl: C, 31.86; H, 4.66; N, 7.96; Cl, 4.03. Found: C, 31.75; H, 4.54; N, 7.68; Cl, 4.05.

### EXAMPLE 50

### AMD8894: Ruthenium (III) complex of N-[2-(N-acetyl-(L)-isoleucyl)]ethylenediamine-N,N′,N′-triacetic acid (aiedta).

N-chloroacetyl-(L)-isoleucine t-butyl ester

At 0° C. under nitrogen, a solution of chloroacetyl chloride (0.64 mL, 8.01 mmol) in anhydrous THF (10 mL) was added dropwise to a suspension of (L)-isoleucine t-butyl ester (1.2 g, 6.41 mmol) and potassium carbonate (1.33 g, 9.62 mmol) in anhydrous THF (10 mL). The reaction mixture was stirred at 0° C. for 30 minutes and then the mixture was evaporated to give a white residue, which was dissolved in a mixture of CH₂Cl₂ and H₂O. The aqueous layer was washed twice with CH₂Cl₂ and then the organic layer was washed twice with H₂O and twice with NH₄Cl (1 N). The combined organic layers were dried (MgSO₄) and evaporated to afford a yellow oil. The crude product was purified by column chromatography on silica gel (5% MeOH/CH₂Cl₂) to afford the desired compound as a yellow oil (0.66 g, 40.9%). ¹H NMR (CDCl₃) δ 0.94 (m, 6H), 1.24 (m, 1H), 1.48 (m, 10H), 1.93 (m, 1H), 4.07 (s, 2H), 4.48 (dd, 1H, J=6.0 Hz, 3.0 Hz), 7.09 (br d, 1H, J=6.0 Hz).

A stirred suspension of N-chloroacetyl-(L)-isoleucine t-butyl ester (0.66 g, 2.62 mmol), potassium carbonate (0.46 g, 3.30 mmol) and ethylenediamine-N,N′,N′-triacetic acid tri-t-butyl ester (0.53 g, 1.31 mmol) in anhydrous acetonitrile (15 mL) was heated to reflux for 60 hours under nitrogen and then evaporated. The light brown residue was partitioned between CH₂Cl₂ and saturated aqueous K₂CO₃. The separated aqueous layer was extracted twice with CH₂Cl₂ and then the combined organic phases were washed twice with K₂CO₃ (saturated) then dried (MgSO₄) and evaporated to give an orange oil. The crude product was purified by centrifugal chromatography on silica gel (CH₂Cl₂ treated with 1% NH₄OH) to afford the desired compound as a yellow oil (0.51 g, 63.4%). ¹H NMR (CDCl₃) δ 0.89 (m, 6H), 1.20 (m, 1H), 1.45 (m, 10H), 1.86 (m, 1H), 2.81 (m, 4H), 3.29 (s, 2H), 3.34 (s, 2H), 3.39 (s, 4H), 4.40 (dd, 1H, J=4.8 Hz), 7.88 (d, 1H, J=9.0 Hz). ¹³C NMR (CDCl₃) δ 12.15, 15.94, 25.63, 28.45, 28.53, 38.18, 53.00, 53.45, 56.48, 56.95, 57.22, 58.89, 81.35, 81.70, 81.80, 170.78, 170.90, 171.04, 171.55.

N-[2-(N-acetyl-(L)-isoleucyl)]ethylenediamine-N,N′,N′-triacetic acid.xTFA (aiedta)

Trifluoroacetic acid (4.0 mL, 51.9 mmol) was added to a solution of the intermediate from above (0.51 g, 0.83 mmol) in anhydrous CH₂Cl₂ (8 mL) and the mixture was stirred overnight at room temperature under nitrogen. The solvent was evaporated and the residue lyophilized to afford a pale yellow solid (0.45 mg, 86%).

¹H NMR (D₂O) δ0.89 (m, 6H), 1.20 (m, 1H), 1.45 (m, 1H), 1.93 (m, 1H), 3.32 (t, 2H, J=6.0 Hz), 3.40 (t, 2H, J=6.0 Hz), 3.82 (s, 2H), 3.88 (s, 2H), 3.96 (s, 4H), 4.33 (d, 1H, J=6.0 Hz). ¹³C NMR (D₂O) δ 11.08, 15.39, 25.05, 36.60, 51.76, 52.03, 55.54, 55.84, 56.64, 58.04, 169.77, 171.49, 172.30, 175.52. ES-MS m/z 406 [M+H]⁺, 428 [M+Na]⁺, 444 [M+K]⁺.

Preparation of [Ru(aiedtaK)(OH₂)1.6H₂O

[Potassium aqua[N-[2-[bis[(carboxy-κO)methyl]amino-κN]ethyl]-N-[(carboxy-κO)methyl]glycyl-κN-L-isoleucinato ruthenium (III)].

Aiedta (0.35 g, 0.55 mmol) was heated in aqueous HCl (1 mM, 5.5 mL) until completely dissolved and the pH of the solution was then adjusted to pH=3.0 with KOH (1N). K₂[RuCl₅(OH₂)] (0.21 g, 0.55 mmol) was added to the solution and the reaction mixture was heated at 100° C. for 2 hours. The solution was evaporated and the residue was purified by size exclusion column chromatography on Sephadex G-10 resin (H₂O). The resulting solid was dried overnight in vacuo at 40° C. to afford the desired complex as a brown crystalline solid (0.030 g, 8.6%). ES-MS m/z 527[M−OH₂−K+Na+H]⁺, 549[M−OH₂−K+2Na]⁺. IR (CsI) ν(cm⁻¹) 1626 (C═O). Anal. Calcd. for C₁₆H₂₅N₃O₁₀RuK.1.6 H₂O.0.6 KCl: C, 30.35; H, 4.49; N, 6.64; Cl, 3.36. Found: C, 30.48; H, 4.64; N, 6.67; Cl, 3.26.

### EXAMPLE 51

### AMD8711: Ruthenium (III) complex of N-benzylethylenediamine-N,N′,N′-triacetic acid (bedta).

N-Benzylethylenediamine-N,N′,N′-triacetic acid tri-t-butyl ester

Following General Procedure E

Reaction of ethylenediamine-N,N′,N′-triacetic acid tri-t-butyl ester (0.734 g, 1.8 mmol) with benzylbromide (0.316 g, 1.8 mmol) afforded, after column chromatography on silica gel (7:1 hexanes: EtOAc), the product (0.496 g, 55%) as a colorless oil.

¹H NMR (CDCl₃) δ 1.40 (s, 18H), 1.42 (s, 9H), 2.80-2.88 (m, 4H), 3.24 (s, 2H), 3.44, (s, 4H), 3.80 (s, 2H), 7.21-7.34 (m, 5H).

N-benzylethylenediamine-N,N′,N′-triacetic acid.xTFA (bedta)

Following General Procedure B

Reaction of the intermediate from above (0.496 g, 1.0 mmol) with TFA (12.6 g, 100 mmol) afforded the product (0.454 g, 82%) as a white solid.

¹H NMR (MeOD) δ 3.10 (t, 2H, J=6.0 Hz), 3.39-3.45 (bs, 6H), 4.09 (s, 2H), 4.59 (s, 2H), 7.47-7.50 (m, 3H), 7.57-7.60 (m, 2H). ¹³C NMR (MeOD) δ 50.59, 53.04, 56.26, 60.90, 130.66, 131.42, 132.01, 132.78, 169.39, 175.74.

Preparation of K[Ru(Hbedta)Cl₂].1.6H₂O

[Potassium Hydrogen aqua[N-[2-[[(carboxy-κO)methyl](carhoxymethyl)amino-κN]ethyl]-N-(phenylmethyl)glycinato-κN,κO]dichloro ruthenium (III)]

Following General Procedure C

Reaction of bedta (0.210 g, 0.38 mmol) with K₂[RuCl₅(H₂O)] (0.142 g, 0.38 mmol) afforded the product (0.0460 g, 21%) as a yellow solid.

Anal. Calcd. for C₁₅H₁₈N₂O₆Cl₂RuK.1.6H₂O.0.1KCl: C, 31.63; H, 3.75; N, 4.92; Cl, 13.07. Found: C, 31.63; H, 3.96; N, 4.77; Cl, 13.03. IR (CsI) ν(cm⁻¹) 1726 (CO₂H); 1641(CO₂₋); 391 (Ru—Cl).

### EXAMPLE 52

### AMD8702: Ruthenium (III) complex of N-[(3-carboxy)benzyl]ethylenediamine-NN′,N′-triacetic acid (cbedta).

N-[(3-carboxy)benzyl]ethylenediamine-N,N′,N′-triacetic acid.xTFA (cbedta) To a stirred solution of N-[(3-carboxymethyl)benzyl]ethylenediamine-N,N′,N′-triacetic acid tri-t-butyl ester (0.771 g, 1.4 mmol) in MeOH (19 mL) and H₂O (6 mL) was added lithium hydroxide (0.236 g, 5.6 mmol) and the reaction was stirred for 16 hours at room temperature (in the absence of light) and then the solvent was evaporated in vacuo. This intermediate was used directly in the next step without further purification.

The residue was dissolved in TFA (8.3 g, 73 mmol) and stirred for 16 hours then evaporated in vacuo. EtOH was added to the residue, the resulting suspension was filtered, and the product lyophilized to afford a white solid (1.04 g, 100%).

¹H NMR (MeOD) δ 3.15 (t, 2H, J=6 Hz), 3.43-3.48 (bs, 6H), 4.09 (s, 2H), 4.64 (s, 2H), 7.59 (dd, 1H, J=6.0, 9.0 Hz), 7.85 (d, 1H, J=6.0 Hz), 8.12 (d, 1H, J=9.0 Hz), 8.26 (s, 1H). ¹³C NMR (MeOD) δ 50.47, 53.65, 54.16, 60.01, 65.74, 130.65, 132.05, 132.30, 133.13, 133.48, 136.67, 168.93, 169.07, 175.12. ES-MS m/z 369 [M+H]⁺.

Preparation of K[Ru(H₂cbedta)Cl₂].4.5H₂O

[Potassium Dihydrogen [3-[[[(carboxy-κO)methyl][2-[[(carboxy-κO))methyl](carboxymethyl) amino-κN]ethyl]amino-κN]methyl]benzoato]dichloro ruthenium (III)]

Following General Reaction C

Reaction of cbedta (0.377 g, 0.60 mmol) with K₂[RuCl₅(H₂O)] (0.236 g, 0.60 mmol) afforded the product (51.0 mg, 12%) as a yellow solid.

Anal. Calcd. for C₁₆H₁₈N₂O₈Cl₂RuK.4.5H₂O.0.1KCl: C, 28.86; H, 4.09; N, 4.21; Cl, 11.18. Found: C, 28.63; H, 3.69; N, 4.29; Cl, 11.08. IR (CsI) ν(cm⁻¹) 1709 (CO₂H); 389 (Ru—Cl).

### EXAMPLE 53

### AMD8849: Ruthenium (III) complex N,N′-bis[2-(N-acetylpyrrolidine)]ethylenediamine-N,N′-diacetic acid (bpedda)

N,N′-bis[2-(N-acetylpyrrolidine)]ethylenediamine-N,N′-diacetic acid (bpedda)

A solution of pyrrolidine (0.56 g, 3.90 mmol) in anhydrous THF (20 mL) was added dropwise to a stirred solution of ethylenediamine-N,N,N′,N′-tetraacetic acid dianhydride (1.0 g, 7.81 mmol) in anhydrous THF (20 mL) under nitrogen and the mixture was stirred for 15.5 hours. The precipitate which formed was collected by filtration and dried in vacuo overnight to give the product as a white solid (1.59 g,˜100%).

¹H NMR (D₂O) δ 1.90 (m, 8H), 3.40 (q, 8H, J=7.2 Hz), 3.52 (s, 4H), 3.83 (s, 4H), 4.13 (s, 4H). ES-MS m/z 399 [M+H]⁺, 421 [M+Na]⁺. Anal. Calcd. for C₁₈H₃₀N₄ O₆.0.2 H₂O: C, 53.77; H, 7.62; N, 13.93. Found: C, 53.68; H, 7.54; N, 13.71.

Preparation of[Ru(bpedda)Cl(OH₂)].3H₂O

[Aquachloro[[N,N′-1,2-ethanediylbis[N-[2-oxo-2-(1-pyrrolidinyl)ethyl]glycinato-κN,κO]]] ruthenium (III)].

Bpedda (0.50 g, 1.26 mmol) was heated in aqueous HCl (1 mM, 10 mL) until completely dissolved. K₂[RuCl₅(OH₂)] (0.47, 1.26 mmol) was added to the solution and the reaction mixture was heated at 100° C. for 2 hours. The solution was filtered and the filtrate was evaporated. The residue was purified by size exclusion column chromatography on Sephadex G-10 resin (H₂O) to afford the desired complex as a red solid (0.039 g, 5.2%). ES-MS mz/z 498 [M—Cl—H₂O]⁺. IR (KBr) ν (cm⁻¹)1626 (C═O).

Anal. Calcd. for C₁₈H₃₀N₄O₇ClRu.3H₂O: C, 35.73; H, 6.00; N, 9.26; Cl, 5.86. Found: C, 35.48; H, 5.50; N, 9.19; Cl, 6.01.

### EXAMPLE 54

### AMD7461: Ruthenium (III) complex of 2-Hydroxy-1,3-propanediamine-N,N,N′,N′-tetraacetic acid (hpdta).

Preparation of [Ru(H₂hpdta)(OH₂)(O₃SCF₃)].EtOH

[Dihydrogen aqua[[N,N′-(2-hydroxy-1,3-propanediyl)bis[N-(carboxymethyl)glycinato-κN,O]]](trifluoromethanesulfonato-κO) ruthenium (III)]

2-Hydroxy-1,3-propanediamine-N,N,N′,N′,-tetraaceticacid (0.082 g, 0.25 mmol) was dissolved in EtOH (20 mL) and [Ru(DMF)₆](OTf)₃ (0.26 g, 0.25 mmol) was added. The reaction was heated to 69° C. for 3 days with stirring, cooled to room temperature and the resulting precipitate was collected by filtration. The solid was washed with EtOH (10 mL) and Et₂O (2×10 mL) to afford the desired product (0.0420 mg, 26%).

Anal. Calcd. for C₁₂H₁₈N₂O₁₃RuF₃S.1.0EtOH: C, 26.50; H, 3.81; N, 4.42. Found: C, 26.60; H, 3.89; N, 4.76. IR (CsI) ν (cm⁻¹) 1744 (CO₂H); 1647 (CO₂₋).

### EXAMPLE 55

### AMD7462: Ruthenium (III) complex of 1,2-Ethylenediamine-N,N′-diaceticacid (edda)

Preparation of K[Ru(edda)CI₂].2.5H₂O

[Potassium dichloro[[N,N′-1,2-ethanediylbis[glycinato-κN,κO]] ruthenium (III)]

1,2-Ethylenediamine-N,N′-diaceticacid (0.130 g, 0.74 mmol) was dissolved in EtOH (20 mL) and RuCl₃.H₂O (0.155 g, 0.74 mmol) added. The mixture was heated to 60° C. during which time a precipitate formed. The solid was collected by filtration and washed with Et₂O to afford the desired product (0.0620 g, 22%) as a brown solid.

Anal. Calcd. for C₆H₁₀N₂O₄Cl₂RuK.2.1H₂O: C, 17.03; H, 3.38; N, 6.62; Cl, 16.76. Found: C, 17.40; H, 3.76; N, 6.80; Cl, 17.20. IR (CsI) ν (cm⁻¹) 1640 (CO₂₋); 318 (Ru—Cl).

### EXAMPLE 56

Synthesis of dithiocarbamate ligands

General Procedure F

Carbon disulfide (1.5-2 equivalents) was dissolved in anhydrous diethyl ether and cooled to 0° C. in an ice bath. The appropriate amine (1 equivalent) and KOH (1-2 equivalents) were dissolved in anhydrous methanol and added dropwise to the carbon disulfide solution. The reaction mixture was stirred for 3 hours at 0° C. The solvent was removed and the resulting residue was triturated with diethyl ether. The white solid was filtered and washed with diethyl ether and dried in vacuo.

The following ligands were prepared using general procedure F:

Pyrrolidinedithiocarbamic acid potassium salt [KS₂CNC₄H₈]

Carbon disulfide (2.16 mL, 36 mmol) was reacted with pyrrolidine (2 mL, 24 mmol) and KOH (1.34 g, 24 mmol) to yield 3.8 g (85%) product.

¹H NMR (D₂O) δ 1.94-1.99 (m, 4H), 3.71-3.76 (m, 4H).

L-Prolinedithiocarbamic acid dipotassium salt [KS₂CNProK]

Carbon disulfide (1.04 mL, 17.4 mmol) was reacted with L-proline (1.0 g, 8.7 mmol) and KOH (0.97 g, 17.4 mmol) to yield 1.37 g (59%) product.

¹H NMR (D₂O) δ 1.950-2.05 (m, 3H), 2.25-2.35 (m, 1H), 3.78-3.96 (m, 2H), 4.84 (m, 1H). ¹³C NMR (D₂O) 624.78, 31.62, 55.77, 69.58, 180.32, 205.71.

L-Prolinemethyl ester dithiocarbamic acid potassium salt [KS₂CNProOMe]

Carbon disulfide (0.53 mL, 8.8 mmol) was reacted with L-proline methyl ester (0.57 g, 4.4 mmol) and KOH (0.49 g, 8.8 mmol) to yield 0.66 g (62%) product. This product contained some residual starting material and was used without further purification in the preparation of the ruthenium complexes.

¹H NMR (D₂O) δ 2.03-2.17 (m, 3H), 2.41-2.44 (m, 1H), 3.78 (m, 1H), 3.91-3.99 (m, 1H), 4.03 (s, 3H), 4.81-4.85 (m, 0.5H), 5.01 (m, 0.5H). ¹³C NMR (D₂O) δ 24.71, 31.02, 53.30, 60.83, 66.79, 175.43, 208.26.

N-Methyl-L-isoleucinedithiocarbamic acid dipotassium salt [KS₂CNMeIleK]

Carbon disulfide (0.83 mL, 13.8 mmol) was reacted with N-methyl-L-isoleucine (1.0 g, 6.89 mmol) and KOH (0.77 g, 13.8 mmol) to yield 0.73 g (37%) product. This product contained some starting material and was used without further purification in the preparation of the ruthenium complexes.

¹H NMR (D₂O) δ 0.91 (t, 3H, J=7.5 Hz), 1.00 (d, 3H, J=6.6 Hz), 1.14-1.23 (m, 1H), 1.30-1.35 (m, 1H), 1.98 (br m, 1H), 3.38 (br s, 3H), 6.01 (d, 1H, J=10.2 Hz).

### EXAMPLE 57

### AMD8672: Preparation of Chloro( 1,4,7-triazacyclononane)bis-(dimethylsufoxide) ruthenium(II) chloride, [Ru(tacn)(DMSO)₂Cl]Cl.

[Chloro[octahydro-1H-1,4,7-triazonine-κN¹,κN⁴,κN⁷]bis[(sulfinyl-κS)bis[methane] ruthenium (II) chloride].

Prepared according to literature procedures: A. Geilenkirchen, P. Neubold, R. Schneider, K. Wieghardt, U. Florke, H-J. Haupt, B. Nuber J Chem. Soc., Dalton Trans. 1994, 457.

### EXAMPLE 58

### AMD8641: Preparation of Trichloro(1,4,7-triazacyclononane) Ruthenium(III): [Ru(tacn)Cl₃].

[Trichloro[octahydro-1H-1,4,7-triazonine-κN¹,κN⁴,κN⁷] ruthenium (III)].

Prepared according to literature procedures: A. Geilenkirchen, P. Neubold, R. Schneider, K. Wieghardt, U. Florke, H-J. Haupt, B. Nuber J. Chem. Soc., Dalton Trans. 1994,457.

### EXAMPLE 59

### AMD8671: Preparation of Trichloro (1,4,7-trimethyl-1,4,7-triazacyclononane) Ruthenium (III): [Ru(Me₃tacn)Cl₃]

[Trichloro[hexahydro-1,4,7-trimethyl-1,4,7-triazonine-κN¹,κN⁴,κN⁷] ruthenium (III)].

Prepared according to literature procedures: P. Neubold, K. Wieghardt, B. Nuber, J. Weiss Inorg Chem. 1989, 28, 459.

### EXAMPLE 60

### AMD8670: Preparation of [Ru(tacn)(S₂CNMe₂)₂][PF₆]

[(Dimethylcarbamodithioato-κS(dimethylcarbamodithioato-κS,κS′) [octahydro-1H-1,4,7-triazonine-κN¹,κN⁴,κN⁷] ruthenium (III) hexafluorophosphate].

General Procedure G

RuLCl₃, where L represents either 1,4,7-triazacyclononane (tacn) or 1,4,7-trimethyl-1,4,7-triazacyclononane (Me₃tacn), was suspended in deionized water and heated to 40° C. Two equivalents of the dithiocarbamic acid salt was added and the reaction continued for 1-1.5 hours during which time the reaction mixture turned a dark blue or purple colour. The reaction mixture was removed from heat and filtered while hot. Saturated NH₄PF₆ was added to the filtrate, which produced a dark precipitate. The solid was filtered and washed with deionized water and diethyl ether and dried in vacuo.

Using General Procedure G

Ru(tacn)Cl₃ (0.30 g, 0.89 mmol) was reacted with N,N-dimethyldithiocarbamic acid sodium salt (NaS₂CNMe₂.2H₂O) (Aldrich, 0.32 g, 1.78 mmol) to yield 0.448 g product (80%).

Anal. Calcd. for C₁₂H₂₆N₅S₄RuPF₆: C, 23.45; H, 4.26; N, 11.39; S, 20.86. Found: C, 23.23; H, 4.34; N, 11.18; S, 20.61. ES-MS m/z 471 [M-PF₆]⁺.

### EXAMPLE 61

### AMD8803: Preparation of [Ru(tacn)(S₂CNEt₂)₂][PF₆]

[(Diethylcarbamodithioato-κS)(diethylcarbamodithioato-κS,κS′) [octahydro-1H-1,4,7-triazonine-κN¹,κN⁴,κN⁷] ruthenium (III) hexafluorophosphate]

Using General Procedure G

Ru(tacn)Cl₃ (0.10 g, 0.29 mmol) was reacted with N,N-diethyldithiocarbamic acid sodium salt (NaS₂CNEt₂.3H₂O) (Aldrich, 0.134 g, 0.6 mmol) to yield 0.163 g product (81%).

Anal. Calcd. for C₁₆H₃₅N₅S₄RuPF₆: C, 28.61; H, 5.25; N, 10.43; S, 10.09. Found: C, 28.44; H, 5.12; N, 10.31; S, 19.30. ES-MS m/z 527 [M-PF₆]⁺.

### EXAMPLE 62

### AMD8842: Preparation of [Ru(tacn)(S₂CNC₄H₈)₂][PF₆]

[(1,4-butanediylcarbamodithioato-κS)(1,4-butanediylcarbamodithioato-κS,κS′)

[octahydro-1H-1,4,7-triazonine-κN¹,κN⁴,κN⁷] ruthenium (III) hexafluorophosphate]

Using General Procedure G

Ru(tacn)Cl₃ (0.10 g, 0.29 mmol) was reacted with pyrrolidinedithiocarbamic acid potassium salt (0.109 g, 0.59 mmol) to yield 0.11 g of crude product. This crude product was purified by column chromatography on silica gel (MeCN/sat. KNO₃/H₂O 7/1/0.5). The solvent was removed from the combined fractions containing the desired product and the residue was triturated with acetonitrile. The excess KNO₃ was removed by filtration and saturated solution of NH₄PF₆ in methanol was added to the filtrate. The resulting precipitate was collected by filtration and washed with deionized water then diethyl ether and dried in vacuo to give the title compound (0.069 g, 36%).

Anal. Calcd. for C₁₆H₃₁N₅S₄RuPF₆.0.2H₂O.0.2NH₄PF₆: C, 27.30; H, 4.61; N, 10.35; S, 18.22. Found: C, 27.06; H. 4.50; N, 10.23; S, 18.24. ES-MS m/z 523 [M-PF₆]⁺.

### EXAMPLE 63

### AMD8731: Preparation of [Ru(tacn)(S₂CNPro)₂][PF₆]

[Dihydrogen ((1-carboxy)-1,4-butanediylcarbamodithioato-κS)((1-carboxy)-1,4-butanediylcarbamodithioato-κS,κS′) [octahydro-1H-1,4,7-triazonine-κN¹,κN⁴,κN⁷] ruthenium (III) hexafluorophosphate]

Using General Procedure G

Ru(tacn)Cl₃ (0.30 g, 0.90 mmol) was reacted with with L-prolinedithiocarbamic acid dipotassium salt (0.48 g, 1.8 mmol) to yield 0.273 g (38%) product.

Anal. Calcd. for C₁₈H₃₁N₅O₄S₄RuPF₆.1.8H₂O: C, 27.43; H, 4.42; N, 8.89; S, 16.27. Found: C, 27.36; H, 4.38; N, 9.07; S, 16.33. ES-MS m/z 611 [M-PF₆]⁺. IR (CsI) ν (cm⁻¹) 1723 (CO₂H).

### EXAMPLE 64

### AMD8802: Preparation of[Ru(tacn)(S₂CNProOMe)₂][PF₆]

((1-carboxymethyl)-1,4-butanediylcarbamodithioato-κS)((1-carboxymethyl)-1,4-butanediylcarbamodithioato-κS,κS′) [octahydro-1H-1,4,7-triazonine-κN¹,κN⁴,κN⁷] ruthenium (III) hexafluorophosphate

Using General Procedure G

Ru(tacn)Cl₃ (0.136 g, 0.40 mmol) was reacted with L-proline methyl ester dithiocarbamic acid potassium salt (0.20 g, 0.80 mmol) to yield 0.078 g (25%) product.

Anal. Calcd. for C₂₀H₃₅N₅O₄S₄RuPF₆: C, 30.65; H, 4.50; N, 8.94; S, 16.35. Found: C, 30.54; H, 4.47; N, 8.81; S, 16.52. ES-MS m/z 639 [M-PF₆]⁺. IR (CsI) ν (cm⁻¹) 1742 (CO₂Me).

### EXAMPLE 65

### AMD8801: Preparation of [Ru(tacn)(S₂CNMelle)₂][PF₆]

[Dihydrogen (N-methyl-N-sec-butylcarboxycarbamodithioato-κS)(N-methyl-N-sec-butylcarboxycarbamodithioato-κS,κS′) [octahydro-1H-1,4,7-triazonine-κN¹,κN⁴,κN⁷] ruthenium (III) hexafluorophosphate]

Using General Procedure G

Ru(tacn)Cl₃ (0.10 g, 0.30 mmol) was reacted with N-methyl-L-isoleucinedithiocarbamic acid dipotassium salt (0.178 g, 0.60 mmol) to yield 0.068 g (28%) product.

Anal. Calcd. for C₂₂H₄₃N₅O₄S₄RuPF₆: C, 32.39; H, 5.31; N, 8.58; S, 15.72. Found: C, 32.41; H, 5.46; N, 8.85; S, 15.58. ES-MS m/z 671 [M-PF₆]⁺. IR (CsI) ν (cm⁻¹) 1726 (CO₂H).

### EXAMPLE 66

### AMD8682: Preparation of [Ru(Me₃tacn)(S₂CNMe₂)₂][PF₆]

[(Dimethylcarbamodithioato-κS)(dimethylcarbamodithioato-κS,κS′) [hexahydro-1,4,7-trimethyl-1,4,7-triazonine-κN¹,κN⁴,κN⁷] ruthenium (III) hexafluorophosphate]

Using General Procedure G

Ru(Me₃tacn)Cl₃ (0.10 g, 0.264 mmol) was reacted with N,N-dimethyldithiocarbamic acid sodium salt (Aldrich, 0.094 g, 0.528 mmol) to yield 0.10 g crude product. This crude product (0.05 g) was purified by column chromatography on silica gel (MeCN/sat. KNO₃/H₂O 7/1/0.5). The solvent was removed from the combined fractions containing the desired product and the residue was triturated with acetonitrile. The KNO₃ was removed by filtration and a saturated solution of NH₄PF₆ in methanol was added to the filtrate. The resulting precipitate was collected, washed with deionized water and diethyl ether and then dried in vacuo to give the title compound (0.030 g, 35%).

Anal. Calcd. for Cl₁₅H₃₃N₅S₄RuPF₆: C, 27.39; H, 5.06; N, 10.65; S, 19.50; Cl, 0.00. Found: C, 27.51; H, 5.01; N, 10.58, S, 19.28; Cl, 0.00. ES-MS m/z 513 [M-PF₆]⁺.

### EXAMPLE 67

### AMD8800: Preparation of[Ru(tacn)(mida)][PF₆]

[(N-(carboxy-κO)-methyl)-N-methylglycinato-κN,κO][octahydro-1H-1,4,7-triazonine-κN¹,κ⁴,κN⁷] ruthenium (III) hexafluorophosphate]

Ru(tacn)Cl₃ (0.10 g, 0.30 mmol) and N-methyliminodiacetic acid (mida) (0.044 g, 0.30 mmol) were refluxed in deionized water (30 mL) for 3 hours. The reaction mixture was filtered hot to remove any unreacted starting material. Saturated aqueous NH₄PF₆ was added to the filtrate and crystallization was induced by the addition of ethanol. The pale yellow precipitate was collected by filtration, washed with diethyl ether and dried in vacuo to yield 0.041 g (26%) product.

Anal. Calcd. for C₁₁H₂₂N₄O₄RuPF₆: C, 25.39; H, 4.26; N, 10.77. Found: C, 25.37; H, 4.24; N, 10.59. ES-MS m/z 376 [M-PF₆]⁺. IR (CsI) ν (cm⁻¹) 1642 (CO₂₋).

### EXAMPLE 68

### AMD8811: Preparation of [Ru(Hnota)Cl]

[Hydrogen chloro[hexahydro-1,4,7-(tricarboxy-κO,κO′-methyl)-1,4,7-triazonine-κN¹,κN⁴,κN⁷] ruthenium (III)]

1,4,7-Triazacyclononane-1,4,7-triacetic acid (nota) (0.50 g, 1 mmol) was dissolved in deionized water (5 mL) and the pH adjusted to pH 3-4 with KOH (1 M). An aqueous solution of K₂[RuCl₅(OH₂)] (0.40 g, 1 mmol) was added to the solution and the reaction mixture was heated to reflux for 2 hours. The solution was cooled and an insoluble material was removed by filtration. Addition of ethanol to the filtrate resulted in the precipitation of [Ru(H₂nota)Cl₂] (0.1 g) which was removed by filtration. Upon allowing the filtrate to stand, a second precipitate was obtained which was collected and washed with diethyl ether to give the title compound (0.040 g, 8.5%).

Anal. Calcd. for C₁₂H₁₉N₃O₆RuCl.H₂O.0.2KCl: C, 30.62; H, 4.50; N, 8.93; Cl, 9.04. Found: C, 30.48; H, 4.64; N, 8.84; Cl, 9.12. ES-MS m/z 403 [M—Cl]⁺. IR (CsI) ν (cm⁻¹) 1728, (CO₂H); 1678 (CO₂₋).

### EXAMPLE 69

### AMD7044: Preparation of [Ru(terpy)(bpy)Cl] [PF₆]

[Chloro(2,2′-bipyridine-κN¹,κN¹′)(2,2′:6′.2″-terpyridine-κN¹,κN²′,κN¹″) ruthenium (II) hexafluorophosphate]

General Procedure H

Terpyridylruthenium trichloride (Ru(terpy)Cl₃) (E. C. Constable et al. New J. Chem. 1992, 16, 855) (0.50 g, 1.13 mmol), bidentate ligand, L (one equivalent) and 4-ethylmorpholine (4 drops) were heated to reflux in methanol (100 mL) for 2 hours. The hot solution was filtered through celite and a saturated solution of NH₄PF₆ in methanol was added to the filtrate. The volume was reduced to approximately one third the original volume at which time a precipitate formed. The crude product was collected by filtration and purified either by re-crystallization from an MeCN/MeOH solution or by column chromatography on silica gel (7/1/0.5: MeCN/sat. KNO₃/H₂O).

Using General Procedure H

Reaction of Ru(terpy)Cl₃ (0.50 g, 1.13 mmol) and 2,2′-dipyridyl (0.18 g, 1.13 mmol) gave the desired product 0.27 g (35%) following purification by column chromatography on silica gel.

¹H NMR (CD₃CN) δ 6.94 (m, 1H), 7.26 (m, 3H), 7.66 (m, 3H), 7.86 (m, 2H), 7.94 (m, 1H), 8.06 (t, 1H, J=7.8 Hz), 8.26 (m, 2H), 8.36 (d, 2H, J=8.1 Hz), 8.47 (d, 2H, J=7.8 Hz), 8.59 (d, 1H, J=8.2 Hz), 10.20 (d, 1H, J=5.8 Hz). ¹³C NMR (CD₃CN) δ 123.4, 124.23, 124.49, 124.57, 127.09, 127.90, 128.25, 134.73, 136.55, 137.54, 138.05, 153.13, 153.25, 153.49, 157.25, 159.01, 159.70, 159.75.

Anal. Calcd. for C₂₅H₁₉N₅CIRuPF₆.0.2NH₄PF₆: C, 42.68; H, 2.84; N, 10.35; Cl, 5.04. Found: C, 42.83; H, 2.61; N, 10.54; Cl, 4.91.

### EXAMPLE 70

### AMD7054: Preparation of [Ru(terpy)(2-pyridinethione)₂Cl][PF₆]

[Chlorobis(2(1H)-pyridinethione-κS²)(2,2′:6′.2″-terpyridine-κN¹,κN²′,κN¹Δ) ruthenium (II) hexafluorophosphate]

Using General Procedure H

Reaction of Ru(terpy)Cl₃ (0.50 g, 1.13 mmol) and 2-mercaptopyridine (0.25 g, 2.27 mmol) gave the desired product (0.263 g, 32%) after re-crystallization from MeCN/MeOH.

¹H NMR (CD₃CN) δ 6.94 (m, 2H), 7.11 (d, 1H, J=7.8 Hz), 7.26 (d, 1H, 5.5 Hz), 7.41 (m, 1H), 7.56 (m, 2H), 7.74 (m, 1H), 7.83 (m, 1H), 8.04-8.21 (m, 5H), 8.28-8.37 (m, 2H), 8.44-8.48 (m, 2H), 9.88 (d, 1H, J=5.5 Hz). ¹³C NMR (CD₃CN) δ 122.04, 123.55, 123.79, 124.03, 124.13, 124.36, 124.60, 125.05, 128.12, 128.41, 137.08, 137.79, 138.29, 139.32, 139.40, 151.45, 152.90, 154.77, 155.61, 156.84, 158.80, 159.12, 159.16, 159.90, 163.65.

Anal. Calcd. for C₂₅H₂₁N₅S₂ClRuPF₆: C, 40.74; H, 2.87; N, 9.50; S, 8.70; Cl, 4.81. Found: C, 40.82; H, 2.80; N, 9.39; S, 8.66; Cl, 4.88.

### EXAMPLE 71

### AMD7055: Preparation of [Ru(terpy)(2-pyrimidinethione)₂Cl][PF₆]

[Chlorobis(2(1H)-pyrimidinethione-κS²)(2,2′:6′.2″-terpyridine-κN¹,κN²′,κN¹″) ruthenium (II) hexafluorophosphate]

Using General Procedure H

Reaction of Ru(terpy)Cl₃ (0.50 g, 1.13 mmol) and 2-mercaptopyrimidine (0.25 g, 2.28 mmol) gave the desired product (0.073g, 8.6%) following purification by column chromatography on silica gel. ¹H NMR (CD₃CN) δ 6.99-7.05 (m, 2H), 7.43 (m, 1H), 7.55-7.60 (M, 2H), 7.81 (m, 1H), 8.10-8.23 (m, 5H), 8.35-8.39 (m, 2H), 8.47-8.50 (m, 2H), 8.87 (dd, 1H, J=4.7, 4.7 Hz), 9.95 (dd, 1H, J=5.9, 2.3 Hz).

Anal. Calcd. for C₂₃H₁₉N₇S₂ClRuPF₆: C, 37.38; H, 2.59; N, 13.27; S, 8.68. Found: C, 38.27; H, 2.39; N, 13.75; S, 8.45.

### EXAMPLE 72

### AMD7086: Preparation of [Ru(terpy)(S₂CNMe₂)Cl][PF₆]

[Chloro(dimethylcarbamodithioato-κS,κS′)(2,2′:6′.2″-terpyridine-κN¹,κN²′,κN¹″) ruthenium (III) hexafluorophosphate]

Ru(terpy)Cl₃ (0.50 g, 1.14 mmol) and N,N-dimethyldithiocarbamic acid sodium salt (Aldrich, 0.204 g, 1.14 mmol) were heated to reflux in methanol (100 mL) for 2 hours. The hot solution was filtered through celite and the volume of the filtrate was reduced to approximately one half the original volume. Addition of a saturated solution of NH₄PF₆ in methanol to the filtrate resulted in the formation of a precipitate, which was collected by filtration and purified by column chromatography on silica gel (MeCN/sat. KNO₃/H₂O: 7/1/0.5) to give the title compound (0.20 g, 28%).

Anal. Calcd. for C₁₈H₁₇N₄S₂ClRuPF₆: C, 34.05; H, 2.70; N, 8.82; S, 10.10. Found: C, 33.76; H, 2.80; N, 9.62; S, 9.95.

### EXAMPLE 73

### AMD7036: Preparation of [Ru(bpy)₂Cl₂].2H₂O

[Dichlorobis(2,2′-bipyridine-κN¹,κN¹′) ruthenium (II) dihydrate]

Prepared according to literature procedures: B. Bosnich, F. P. Dwyer Aust. J. Chem. 1966, 19, 2229.

### EXAMPLE 74

### AMD7037: Preparation of [Ru(phen)₂Cl_{2].2}H₂O

[Dichlorobis(1,10-phenanthroline-κN¹,κN¹⁰) ruthenium (II) dihydrate]

Prepared according to literature procedures: B. Bosnich, F. P. Dwyer Aust. J. Chem. 1966, 19, 2229.

### EXAMPLE 75

### AMD)7039: Preparation of [Ru(bpy)₂(2-mercaptopyridine)][ClO₄]

[Bis(2,2′-bipyridine-κN¹,κN¹′)(2(1H)-pyridinethionato-κN¹,κS²) ruthenium (II)]Perchlorate.

Prepared according to literature procedures: B. Kumar Santra, M. Menon, C. Kumar Pal, G. Kumar Lahiri J. Chem. Soc., Dalton Trans. 1997, 1387.

### EXAMPLE 76

### AMD7045: Preparation of [Ru(bPY)₂(2-mercaptopyridine)][PF₆]

[Bis(2,2′-bipyridine-κN¹,κN¹′)(2(1H)-pyridinethionato-κN¹,κS²) ruthenium (II) hexafluorophosphate]

[RU(bpy)₂Cl₂].2H₂O (1.0 g, 1.9 mmol) was dissolved in a 1: 1 mixture of methanol water (100 mL). 2-Mercaptopyridine was added to the solution and the reaction mixture was heated to reflux for 1.5 hours. The solution was cooled to room temperature and a saturated solution of NH₄PF₆ in methanol was added. Upon standing a dark purple precipitate formed which was removed by filtration and washed with water. This crude product was purified by column chromatography on silica gel (2:1 wCHCl₃MeCN) to give the title compound (0.92 g, 72%).

¹H NMR (CD₃CN) δ 6.58-6.27 (m, 1H), 6.76 (d, 1H, J=8.16 Hz), 7.00-7.02 (m, 1H), 7.13-7.17 (m, 1H), 7.19-7.23 (m, 1H), 7.29-7.34 (m, 1H), 7.55-7.60 (m, 1H), 7.67-7.89 (m, 5H), 8.04 (t, 2H, J=7.9 Hz), 8.25 (d, 1H, J=5.2 Hz), 8.36 (t, 2H, J=8.2 Hz), 8.46 (t, 2H, J=7.3 Hz), 9.84-9.86 (m, 1H).

Anal. Calcd. for C₂₅H₂₀N₅SRuPF₆: C, 44.91; H, 3.02; N, 10.48; S, 4.80. Found: C, 44.88; H, 3.02; N, 10.58; S, 4.71.

### EXAMPLE 77

### AMD8657: Synthesis of [Ru(acac)₂(MeCN)₂][CF₃SO₃]

[Bis(acetonitrile)bis(2,4-pentanedionato-κO,κO′) ruthenium (III) trifluoromethanesulfonate].

General Procedure I

This synthesis was adapted from a literature procedure: Oomura, K.; Ooyama, D.; Satoh, Y.; Nagao, N.; Nagao, H.; Howell, M.; Mukaida, M. Inorg. Chim. Acta 1998, 269, 342. In a schlenk tube, Ru(β-diketonato)₃ was dissolved in acetonitrile (˜1 g/50 mL) and the mixture was stirred for 5 min at 65° C. to yield a orange/red/purple solution; Trifluoromethanesulfonic acid (1.1-4 equivalents) was then added dropwise. Instantly, the solution became brown/green; a reflux condenser was then attached and the mixture was heated to reflux for 0.5-4 h. The final navy blue (Ru(III)) and/or orange/red/brown (Ru(II)) mixture was concentrated and purified by crystalization or column chromatography. tris-(2,4-pentanedionato) ruthenium(III) [Ru(acac)₃] was Prepared according to procedure adapted from the literature: Johnson, A.; Everett, Jr., G. W. J. Am. Chem. Soc. 1972, 94, 1419.

Preparation of [Ru(acac)₂(MeCN)₂][CF₃SO₃]

Using General Procedure I

Ru(acac)₃ (1.07 g, 2.68 mmol) was dissolved in acetonitrile (50 mL). Addition of Trifluoromethanesulfonic acid (300 μL, 3.39 mmol) yielded the title complex after stirring for 1 h at reflux; crystallization from a 40:1 mixture of Et₂O:CH₂Cl₂ at 5° C. overnight yielded a dark blue, crystalline solid (1.42 g, 96%).

Anal. Calcd. for C₁₅H₂₀N₂O₇SF₃Ru.H₂O: C, 31.85; H, 3.91; N, 3.98. Found: C, 32.13; H, 3.87; N, 3.96. ES-MS m/z 382 [M—CF₃SO₃]⁺. IR (KBr) ν (cm⁻¹) 2326, 2296 (C≡N); 1524 (C≡O).

### EXAMPLE 78

### AMD8660: Synthesis of Ru(acac)₂(MeCN)₂

[Bis(acetonitrile)bis(2,4-pentanedionato-κO,κO′) ruthenium (II)]

Preparation of Ru(acac)₂(MeCN)₂

[Ru(acac)₂(MeCN)₂][CF₃SO₃] (0.201 g, 0.378 mmol) was dissolved in EtOH (10 mL) to give a blue solution. Addition of Me₂NCS₂Na.2H₂O (0.076 g, 0.426 mmol) afforded an orange/brown solution immediately. The mixture was stirred at room temperature for 5 min and then the solvent was removed under reduced pressure. The orange/brown residue was purified by column chromatography on silica gel; 20:1 CH₂Cl₂:MeOH). The major orange band was collected in several fractions and the solvent removed under reduced pressure to yield a yellow/orange solid (0.094 g, 65%).

Anal. Calcd. for C₁₄H₂₀N₂O₄Ru.0.5C₂H₆O: C, 37.89; H, 5.18; N, 3.19. Found: C, 38.01; H, 4.99; N, 3.26. ES-MS m/z 382 [M+H]⁺. IR (KBr) ν (cm⁻¹) 2333, 2251 (C≡N); 1566 (C═O).

### EXAMPLE 79

### AMD8892: Synthesis of [Ru(3Meacac)₂(MeCN)₂][CF₃SO₃]

[Bis(acetonitrile)bis(3-methyl-2,4-pentanedionato-κO,κO′) ruthenium (III) trifluoromethanesulfonate]

tris-(3-methyl-2,4-pentanedionato) ruthenium(I) [Ru(3Meacac)₃] was prepared according to literature procedures: Endo, A.; Shimizu, K.; Satô, G. P. Chem. Lett. 1985, 581.

Preparation of [Ru(3Meacac)₂(MeCN)₂][CF₃SO₃]

Using General Procedure I

Ru(3Meacac)₃ (0.522 g, 1.19 mmol) was dissolved in acetonitrile. Addition of Trifluoromethanesulfonic acid (115 μL, 1.31 mmol) yielded the title complex after 1 h at reflux; crystallization from a 40:1 mixture of Et₂O:CH₂Cl₂ at 5° C. overnight yielded a dark blue, crystalline solid (0.608 g, 92%).

Anal. Calcd. for C₁₇H₂₄N₂O₇SF₃Ru: C, 36.56; H, 4.33; N, 5.02; S, 5.74. Found: C, 36.29; H, 4.34; N, 5.04; S, 5.86. ES-MS m/z 410 [M—CF₃SO₃]⁺. IR (KBr) ν (cm⁻¹) 2316, 2296 (C≡N); 1535 (C═O).

### EXAMPLE 80

### AMD8901: Synthesis of Ru(3Meacac)₂(MeCN)₂

[Bis(acetonitrile)bis(3-methyl-2,4-pentanedionato-κO,κO′) ruthenium (II)]

Preparation of Ru(3Meacac)₂(MeCN)₂

[Ru(3Meacac)₂(MeCN)₂][CF₃SO₃] (0.105 g, 0.188 mmol) was dissolved in acetonitrile (25 mL) to give a blue solution. Addition of zinc shavings (˜12 g) followed by rapid stirring for 4 h at room temperature led to the formation of a bright orange solution. The zinc was removed by filtration, the solvent concentrated in vacuo and then the mixture was purified by column chromatography on silica gel; 20:1 CH₂Cl₂:MeOH). The major orange band was collected in several fractions and the solvent removed under reduced pressure to yield a bright orange solid (0.025 g, 32%).

Anal. Calcd. for C₁₆H₂₄N₂O₄Ru.0.1CH₂Cl₂: C, 46.27; H, 5.84; N, 6.70. Found: C, 46.00; H, 5.81; N, 6.43. ES-MS m/z 410 [M+H]⁺. IR (KBr) ν (cm⁻¹) 2336, 2248 (C≡N); 1555 (C═O).

### EXAMPLE 81

### AMD8883 and AMD8884: Synthesis of Ru(3Clacac)₂(MeCN)₂ and [Ru(3Clacac)₂(MeCN)₂][CF₃SO₃]

[Bis(acetonitrile)bis(3-chloro-2,4-pentanedionato-κO,κO′) ruthenium (II)] and [Bis(acetonitrile)bis(3-chloro-2,4-pentanedionato-κO,κO′) ruthenium (III) trifluoromethanesulfonate].

tris-(3-chloro-2,4-pentanedionato) ruthenium(III) [Ru(3Clacac)₃] was prepared according to literature procedure: Endo, A.; Shimizu, K.; Satô, G. P. Chem. Lett. 1985, 581.

Preparation of Ru(3Clacac)₂(MeCN)₂ and [Ru(3Clacac)₂(MeCN)₂][CF₃SO₃]

Using General Procedure I

Ru(3Clacac)₃ (0.375 g, 0.745 mmol) was dissolved in acetonitrile (25 mL). Trifluoromethanesulfonic acid (220 μL, 2.48 mmol) was added and the mixture was heated to reflux for 1 h; purification by column chromatography on silica gel (20:1 CH₂Cl₂:MeOH) resulted in the isolation of two major bands (orange and blue). The fractions containing the orange band were concentrated to ˜5 mL and hexanes were added to give a bright orange precipitate of Ru(II)(3Clacac)₂(MeCN)₂ which was isolated via suction filtration (0.085 g, 25%).

Anal. Calcd. for C₁₄H₁₈N₂O₄Cl₂Ru.0.4CH₂Cl₂: C, 35.64; H, 3.91; N, 5.76; Cl, 20.72. Found: C, 35.91; H, 4.07; N, 5.61; Cl, 21.00. ES-MS m/z 452 [M+H]⁺. IR (KBr) ν (cm⁻¹) 2335, 2261 (C≡N); 1543 (C═O).

The fractions containing the blue band were concentrated and the dark blue product was crystallized from a 40:1 mixture of Et₂O:CH₂Cl₂ at 5° C. overnight to give [Ru(III)(3Clacac)₂(MeCN)₂][CF₃SO₃] (0.155 g, 35%).

Anal. Calcd. for C₁₅H₁₈N₂O₇Cl₂SF₃Ru.0.1C₄H₁₀O: C, 30.48; H, 3.16; N, 4.62; S, 5.28; Cl, 11.69. Found: C, 30.56; H, 3.28; N, 4.77; S, 5.29; Cl, 11.70. ES-MS m/z 451 [M—CF₃SO₃]⁺. IR (KBr) ν (cm⁻¹) 2326, 2298 (C≡N); 1532 (C═O).

### EXAMPLE 82

### AMD8881: Synthesis of [Ru(3Bracac)₂(MeCN)₂][CF₃SO₃]

[Bis(acetonitrile)bis(3-bromo-2,4-pentanedionato-κO,κO′) ruthenium (III) trifluoromethanesulfonate]

tris-(3-bromo-2,4-pentanedionato) ruthenium(III) [Ru(3Bracac)₃] was prepared according to literature procedures: Endo, A.; Shimizu, K.; Satô, G. P. Chem. Lett. 1985, 581.

Preparation of [Ru(3Bracac)₂MeCN)₂][CF₃SO₃]

Using General Procedure I

Ru(3Bracac)₃ (0.638 g, 1.00 mmol) was dissolved in acetonitrile (25 mL). Addition of Trifluoromethanesulfonic acid (265 μL, 2.99 mmol) yielded the title complex after 1 h at reflux; the mixture was purified by column chromatography on silica gel (20:1 CH₂Cl₂:MeOH) followed by crystallization from a 40:1 mixture of Et₂O:CH₂Cl₂ at 5° C. overnight, to give a dark blue crystalline solid (0.315 g, 46%).

Anal. Calcd. for C₁₅H₁₈N₂O₇Br₂SF₃Ru.0.3 C₄H₁₀O: C, 27.39; H, 2.98; N, 3.94; S, 4.51. Found: C, 27.62; H, 2.69; N, 4.25; S, 4.70. ES-MS m/z 539 [M—CF₃SO₃]⁺. IR (KBr) ν (cm⁻¹) 2326, 2299 (C≡N_{sym.}); 1522 (C═O).

### EXAMPLE 83

### AMD8900: Synthesis of Ru(3Bracac)₂(MeCN)₂

[Bis(acetonitrile)bis(3-bromo-2,4-pentanedionato-κO,κO′) ruthenium (II)]

Preparation of Ru(3Bracac)₂(MeCN)₂

[Ru(3Bracac)₂(MeCN)₂][CF₃SO₃] (0.350 g, 0.508 mmol) was dissolved in acetonitrile (50 mL) to give a blue solution. Addition of basic alumina (˜15 g) followed by rapid stirring for 2 h at room temperature resulted in the formation of an orange/brown solution. The alumina was removed by filtration, the solvent concentrated under reduced pressure and the residue was purified by column chromatography on silica gel (20:1 CH₂Cl₂:MeOH). The major orange band was collected in several fractions and the solvent was removed under reduced pressure. The orange residue was recrystallized from acetone:hexanes to yield a bright orange solid (0.115 g, 42%).

Anal. Calcd. for C₁₄H₁₈N₂O₄Br₂Ru.0.3C₃H₆O: C, 32.76; H, 3.72; N, 4.93; Br, 28.12. Found: C, 32.74; H, 3.74; N, 4.96; Br, 28.23. ES-MS m/z 540 [M+H]⁺. IR (KBr) ν(cm⁻¹) 2340, 2263 (C≡N); 1530 (C═O).

### EXAMPLE 84

### AMD8910 and AMD8896: Synthesis of [Ru(3Iacac)(acac)(MeCN)₂][CF₃SO₃] and [Ru(3Iacac)(MeCN)₄][CF₃SO₃]

[Bis(acetonitrile)(2,4-pentanedionato-κO,κO′)(3-iodo-2,4-pentanedionato-κO,κO′) ruthenium (III) trifluoromethanesulfonate] and

[Tetrakis(acetonitrile)(3-iodo-2,4-pentanedionato-κO,κO′) ruthenium (II) trifluoromethanesulfonate].

tris-(3-iodo-2,4-pentanedionato) ruthenium(III) [Ru(3Iacac)₃] was prepared according to literature procedures: Endo, A.; Shimizu, K.; Satô, G. P. Chem. Lett. 1985, 581.

Preparation of [Ru(3Iacac)₂(MeCN)₂][CF₃SO₃] and [Ru(3Iacac)(MeCN)₄CF₃SO₃]

Using General Procedure I

Ru(3Iacac)₃ (0.460 g, 0.593 mmol) was dissolved in acetonitrile (25 mL). Trifluoromethanesulfonic acid (60 μL, 0.678 mmol) was added and the reaction was heated to reflux for 1 hour; the reaction mixture was purified by column chromatography on silica gel (15:1 CH₂Cl₂:MeCN) to give [Ru(3Iacac)(acac)(MeCN)₂][CF₃SO₃] as a dark blue crystalline solid (0.089 g, 30%).

Anal. Calcd. for C₁₅H₁₉N₂O₇ISF₃Ru: C, 27.45; H, 2.92; N, 4.27; S, 4.88; I, 19.33. Found: C, 27.35; H, 3.00; N, 4.21; S, 4.91; I, 19.46. ES-MS m/z 508 [M—CF₃SO₃]⁺. IR (KBr) ν (cm⁻¹) 2326, 2297, 2249 (C≡N), 1523 (C═O).

Repeating the above procedure with 4 equivalents of Trifluoromethanesulfonic acid followed by silica gel column purification and recrystallization of the product from acetone:hexanes gave [Ru(3Iacac)(MeCN)₄][CF₃SO₃] as a grey/purple crystalline solid (0.125 g, 33%).

Anal. Calcd. for C₁₄H₁₈N₄O₅ISF₃Ru.0.7 C₃H₆O: C, 28.44; H, 3.29; N, 8.24; S, 4.71. Found: C, 28.12; H, 3.20; N, 8.02; S, 4.39. ES-MS m/z 491 [M—CF₃SO₃]⁺. IR (KBr) ν (cm⁻¹) 2339, 2284 (C≡N), 1537 (C═O).

### EXAMPLE 85

### AMD8691: Synthesis of [Ru(dpac)₂(MeCN)₂][CF₃SO₃]

[Bis(acetonitrile)bis(1,3-diphenyl-1,3-propanedionato-κO,κO′) ruthenium (III) trifluoromethanesulfonate]

tris-(1,3-diphenyl-1,3-propanedionato) ruthenium(III) [Ru(dpac)₃] was prepared according to procedures adapted from the literature: Endo, A.; Shimizu, K.; Satô, G. P.; Mukaida, M. Chem. Lett. 1984,437.

Preparation of [Ru(dpac)₂(MeCN)₂][CF₃O₃]

Using General Procedure I

Ru(dpac)₃ (8.103 g, 10.5 mmol) was dissolved in acetonitrile (250 mL). Trifluoromethanesulfonic acid (2.5 mL, 28.2 mmol) was added and the reaction mixture was heated to reflux for 20 mins. The mixture was evaporated to dryness and the residue was purified by column chromatography on silica gel (CH₂Cl₂→20:1 CH₂Cl₂:MeOH). The fractions containing the dark green band were combined and evaporated to give a dark green crystalline solid (5.75 g, 70%).

Anal. Calcd. for C₃₅H₂₈N₂O₇SF₃Ru.0.4H₂O: C, 53.49; H, 3.69; N, 3.56; S, 4.08. Found: C, 53.45; H, 3.74; N, 3.43; S, 3.97. ES-MS m/z 630 [M—CF₃SO₃]⁺. IR (KBr) ν (cm⁻¹) 2363, 2337 (C≡N); 1523 (C═O).

### EXAMPLE 86

### AMD8692: Synthesis of Ru(dpac)₂(MeCN)₂

[Bis(acetonitrile)bis(1,3-diphenyl-1,3-propanedionato-κO,κO′) ruthenium (II)]

Preparation of Ru(dpac)₂(MeCN)₂

[Ru(dpac)₂(MeCN)₂][CF₃SO₃] (0.225 g, 0.289 mmol) was dissolved in CH₂Cl₂ (25 mL) to give a green solution. Addition of basic alumina (˜10 g) resulted in an instant colour change to orange. The mixture was stirred for 30 min at room temperature, the alumina was removed by filtration and the filtrate was evaporated to dryness to yield a bright orange solid (0.045 g, 25%).

Anal. Calcd. for C₃₀H₂₈N₂O₄Ru.0.5H₂O: C, 64.01; H, 4.57; N, 4.39. Found: C, 64.02; H, 4.58; N, 4.19. ES-MS m/z 630 [M+H]⁺. IR (KBr) ν (cm⁻¹) 2339, 2258 (C≡N), 1516 (C═O).

### EXAMPLE 87

### AMD8707: Synthesis of [Ru(hmac)₂(MeCN)₂][CF₃SO₃]

[Bis(acetonitrile)bis(2,2,6,6-tetramethyl-3,5-heptanedionato-κO,κO′) ruthenium (III) trifluoromethanesulfonate]

tris-(2,2,6,6-tetramethyl-3,5-heptanedionato) ruthenium(III) [Ru(hmac)₃] was prepared according to literature procedures: Endo, A.; Katjitani, M.; Mukaida, M.; Shimizu, K.; Satô, G. P. Inorg. Chim. Acta 1988,150, 25.

Preparation of [Ru(hmac)₂(MeCN)₂][CF₃SO₃]

Using General Procedure I

Ru(hmac)₃ (0.145 g, 0.207 mmol) was dissolved in acetonitrile (10 mL). Trifluoromethanesulfonic acid (40 μL, 0.452 mmol) was added and the mixture was heated to reflux for 30 mins. The mixture was evaporated to dryness and the residue was purified by column chromatography on silica gel (CH₂Cl₂: hexanes 1:1 followed by 20:1 CH₂Cl₂:MeOH). The fractions containing the blue band were combined and evaporated to give a dark blue crystalline solid (0.104 g, 67%).

Anal. Calcd. for C₂₇H₄₄N₂O₇SF₃-Ru.1.6CH₄O: C, 45.79; H, 6.78; N, 3.73. Found: C, 45.86; H, 6.62; N, 3.34. ES-MS m/z 550 [M—CF₃SO₃]⁺. IR (KBr) ν (cm⁻¹) 2326, 2297 (C≡N); 1529 (C═O).

### EXAMPLE 88

### AMD8658: Synthesis of Ru(hfac)₂(MeCN)₂

[Bis(acetonitrile)bis(1,1,1,5,5,5-hexafluoro-2,4-pentanedionato-κO,κO′) ruthenium (II)]

tris-(1,1,1,5,5,5-hexafluoro-2,4-pentanedionato) ruthenium(III) [Ru(hfac)₃]. The ruthenate complex, K[Ru(hfac)₃], was isolated and then oxidized to Ru(hfac)₃ according to a literature procedure: Endo, A.; Katjitani, M.; Mukaida, M.; Shimizu, K.; Satô, G. P. Inorg Chim. Acta 1988,150, 25.

Preparation of Ru(hfac)₂(MeCN)₂

Using General Procedure I

Ru(hfac)₃ (4.00 g, 5.54 mmol) was dissolved in acetonitrile (200 mL). Trifluoromethanesulfonic acid (865 μL, 6.06 mmol) was added and the mixture was heated to reflux for 1 hour. The solvent was evaporated and the residue was purified by column chromatography on silica gel (CH₂Cl₂) to give a browniblack crystalline solid (2.71 g, 95%).

Anal. Calcd. for C₁₄H₈N₂O₄F₁₂Ru: C, 28.15; H, 1.35; N, 4.69. Found: C, 28.35; H, 1.33; N, 4.62. ES-MS m/z 598 [M+H]⁺. IR (KBr) ν (cm⁻¹) 2357, 2285 (C≡N), 1546 (C═O).

### EXAMPLE 89

### AMD8693 and AMD8694: Synthesis of sym and asym-Ru(tfac)₂(MeCN)₂

[sym-Bis(acetonitrile)bis(1,1,1-trifluoro-2,4-pentanedionato-κO,κO′) ruthenium (II)] and [asym-Bis(acetonitrile)bis(1,1,1-trifluoro-2,4-pentanedionato-κO,κO′) ruthenium (II)].

tris-(1,1,1-trifluoro-2,4-pentanedionato) ruthenium(III) [Ru(tfac)₃] was prepared according to literature procedures (a mixture of Δ and Λ-isomers isolated): Endo, A.; Katjitani, M.; Mukaida, M.; Shimizu, K.; Satô, G. P. Inorg. Chim. Acta 1988, 150, 25.

Synthesis of sym and asym-Ru(tfac)₂(MeCN)₂

Following General Procedure I

A mixture of Δ and Λ-Ru(tfac)₃ (1.57 g, 2.80 nimol) in acetonitrile (100 mL). Trifluoromethanesulfonic acid (500 μL, 3.50 mmol) was added and the mixture was heated to reflux for 4 hours during which time the solution turned purple/blue. Addition of basic alumina (˜50 g) afforded an orange solution containing a mixture of the title complexes. The alumina was removed via filtration and the filtrate was purified by column chromatography on silica gel (20:1 CH₂Cl₂:MeOH) to give three bands which eluted in the following order: sym-Ru(tfac)₂(MeCN)₂, sym/asym-Ru(tfac)₂(MeCN)₂ mixture and asym-Ru(tfac)₂(MeCN)₂. Each fraction was evaporated to dryness to give orange solids; the yields of each compound after recrystallization from acetone/hexanes were: 0.121 g, 0.319 g and 0.244 g, respectively, affording an overall yield of 48%. Both pure isomers have essentially identical analytical data.

Anal. Calcd. for C₁₄H₁₄N₂O₄F₆Ru.1.3C₃H₆O: C, 38.11; H, 3.90; N, 4.95. Found: C, 38.29; H, 3.24; N, 4.97. ES-MS m/z 490 [M+H]⁺. IR (KBr) ν (cm⁻¹) 2345, 2270 (C≡N); 1591 (C═O).

### EXAMPLE 90

### AMD8730 and AMD8710: Synthesis of sym and asym-Ru(tftmac)₂(MeCN)₂

[sym-Bis(acetonitrile)bis(1,1,1-trifluoro-5,5-dimethyl-2,4-hexanedionato-κO,κO′) ruthenium (II)] and [asym-Bis(acetonitrile)bis(1,1,1-trifluoro-5,5-dimethyl-2,4-hexanedionato-κO,κO′) ruthenium (II)].

tris-(1,1,1-trifluoro-5,5-dimethyl-2,4-hexanedionato) ruthenium(III) [Ru(tftmac)₃] was prepared according to literature procedure (a mixture of Δ and Λ-isomers isolated): Endo, A.; Katjitani, M.; Mukaida, M.; Shimizu, K.; Satô, G. P. Inorg Chim. Acta 1988, 150, 25.

Preparation of sym and asym-Ru(tftmac)₂(MeCN)₂

Using General Procedure I

A mixture of Δand Λ-Ru(tftmac)₃ (1.30 g, 1.89 mmol) was dissolved in acetonitrile (100 mL). Trifluoromethanesulfonic acid (425 μL, 2.97 mmol) was added and the reaction mixture was heated to reflux for 3 hours during which time the solution turned purple. Addition of basic alumina (˜35 g) afforded an orange solution containing a mixture of the title complexes after stirring for 1.5 h at room temperature. The alumina was removed by filtration and the filtrate was purified by column chromatography on silica gel (CH₂Cl₂). Two compounds were isolated which eluted in the order: sym-Ru(tftmac)₂(MeCN)₂ followed by asym-Ru(tftmac)₂(MeCN)₂. The fractions collected were evaporated to yield orange solids, which were recrystallized from acetone/hexanes to give 0.098 g and 0.461 g, respectively, affording an overall yield of 64%. Both pure isomers have essentially identical analytical data.

Anal. Calcd. for C₂₀H₂₆N₂O₄F₆Ru.0.5C₃H₆O: C, 42.86; H, 4.85; N, 4.65. Found: C, 42.93; H, 4.60; N, 4.77. ES-MS m/z 574 [M+H]⁺. IR (KBr) ν (cm⁻¹) 2330, 2268 (C≡N); 1591 (C═O).

### EXAMPLE 91

### AMD8757: Synthesis of [Ru(maltol)₂(MeCN)₂][CF₃SO₃]

[Bis(acetonitrile)bis[(3-hydroxy-κO)-2-methyl-4-pyronato-κO′] ruthenium (III) trifluoromethanesulfonate]

Preparation of[Ru(maltol)₂(MeCN)₂][CF₃SO₃]

Following General Procedure I

Ru(maltol)₃ (0.210 g, 0.441 mmol) was dissolved in acetonitrile (20 mL). Trifluoromethanesulfonic acid (50 μL, 0.565 mmol) was added and the reaction mixture was heated to reflux for 3 hours. The mixture was evaporated and the residue was purified by column chromatography on silica gel (10:1 CH₂Cl₂: MeOH). The fractions containing the dark green band were combined and evaporated and the residue was then recrystallized from acetone/hexanes to give a dark green crystalline solid (0.085 g, 35%).

Anal. Calcd. for C₁₇H₁₆N₂O₉SF₃Ru.0.4C₃H₆O: C, 36.09; H, 3.06; N, 4.63. Found: C, 36.06; H, 3.09; N, 4.44. ES-MS m/z 434 [M—CF₃SO₃]⁺. IR (KBr) ν (cm⁻¹) 2322, 2289 (C≡N), 1602, 1548 (C═O).

### EXAMPLE 92

### AMD8695 and AMD8696: Synthesis of [Ru(acac)₂(MeCN)₂(tmpd)][CF₃SO₃] and [Ru(acac)₂(MeCN)₂(tmpd)₂][CF₃SO₃

[Bis(acetonitrile)bis[4-(hydroxy-κO)-3-penten-2-onato](N,N,N′,N′-tetramethyl-1,3-propanediamine-κN,κN′) ruthenium (III) trifluoromethanesulfonate] and

[Bis(acetonitrile)bis[4-(hydroxy-κO)-3-penten-2-onato]bis(N,N,N′,N′-tetramethyl- 1,3-propanediamine-κN) ruthenium (III) trifluoromethanesulfonate]

General Procedure J

In a schlenk tube, [Ru(acac)₂(MeCN)₂][CF₃SO₃] was dissolved in CH₂Cl₂to give a blue solution Dropwise addition of an amine ligand, resulted in an immediate colour change to red/orange. The mixture was stirred at 40° C. for 0.5-3 h before the solvent was removed under reduced pressure and the red/brown residue was purified by column chromatography on silica gel. The amine ligands used included: N,N,N′,N′-tetramethyl-1,3-propanediamine (tmpd), diethylenetriamine (dien), 2-(2-aminoethylamino)ethanol (aeae), N-(2-aminoethyl)-1,3-propanediamine (aepd), N-(3-aminopropyl)-1,3-propanediamine (appd), and L1.

Preparation of [Ru(acac)₂(MeCN)₂(tmpd)][CF₃SO₃] and [Ru(acac)₂(MeCN)₂(tmpd)₂][CF₃SO₃]

Using General Procedure J

Addition of tmpd (135 μL, 0.807 mmol) to a CH₂Cl₂ solution of [Ru(acac)₂(MeCN)₂][CF₃SO₃] (0.353 g, 0.665 mmol) afforded a red/orange solution after 1.5 hours. The mixture was purified by column chromatography on silica gel (20:1 CH₂Cl₂:MeOH) to give a red product and an orange product. The fractions from the red and orange bands were evaporated to give dark red (0.039 g, 9%) and bright orange (0.069 g, 13%) solids, respectively. The red solid was characterized as [Ru(acac)₂(MeCN)₂(tmpd)][CF₃SO₃].

Anal. Calcd. for C₂₂H₃₈N₄O₇SF₃Ru.1.3CH₂Cl₂: C, 36.25; H, 5.30; N, 7.25. Found: C, 36.18; H, 5.29; N, 7.46. ES-MS m/z 512 [M—CF₃SO₃]⁺. IR (KBr) ν (cm⁻¹) 2361, 2340 (C≡N); 1620, 1524 (C═O). The orange solid was characterized as [Ru(acac)₂(MeCN)₂ (tmpd)₂][CF₃SO₃].

Anal. Calcd. for C₂₉H₅₆N₆O₇SF₃Ru.1.8CH₂Cl₂: C, 39.27; H, 6.38; N, 8.93. Found: C, 39.18; H, 6.39; N, 9.17. ES-MS m/z 642 [M—CF₃SO₃]⁺. IR (KBr) ν (cm⁻¹) 2300 (C≡N); 1624, 1608, 1548, 1521 (C═O).

### EXAMPLE 93

### AMD8704 and AMD8705: Synthesis of sym and asym-[Ru(acac)₂(MeCN)₂(dien)] [CF₃SO₃]

[Bis(acetonitrile)[N,N′-bis[2-(amino-κN)ethyl]amine]bis[4-(hydroxy-κO)-3-penten-2-onato] ruthenium (III) trifluoromethanesulfonate] and [Bis(acetonitrile)[N-(2-aminoethyl)-1,2-ethanediamine-κN,κN′]bis[4-(hydroxy-κO)-3-penten-2-onato] ruthenium (III) trifluoromethanesulfonate].

Preparation of sym and asym-[Ru(acac)₂(MeCN)₂(dien)][CF₃SO₃]

Following General Procedure J

Addition of dien (70 μL, 0.613 mmol) to a CH₂Cl₂ solution of [Ru(acac)₂(MeCN)₂][CF₃SO₃] (0.325 g, 0.613 mmol) afforded a red/orange solution after 1 hour. The volume was reduced to 5 mL and Et₂O (˜50 mL) was added to give an orange/brown precipitate which was removed via filtration. The brilliant orange filtrate was concentrated under reduced pressure and the residue was purified by column chromatography on silica gel (20:1→12:1 CH₂Cl₂: MeOH). The first orange band afforded a bright orange solid (0.048 g, 12%) whose characterisation data was consistent with the structure asym-[Ru(acac)₂(MeCN)₂(dien)][CF₃SO₃].

Anal. Calcd. for C₁₉H₃₃N₅O₇SF₃Ru: C, 36.02; H, 5.25; N, 11.05. Found: C, 35.75; H, 5.18; N, 10.78. ES-MS m/z 485 [M—CF₃SO₃]⁺. IR (KBr) ν (cm⁻¹) 1628, 1514 (C═O).

The second orange band afforded an orange solid (0.035 g, 9%) whose characterisation data was consistent with the structure sym-[Ru(acac)₂(MeCN)₂(dien)] [CF₃SO₃].

Anal. Calcd. for C₁₉H₃₃N₅O₇SF₃Ru.3.6CHCl₃: C, 25.50; H, 3.46; N, 6.58. Found: C, 25.44; H, 3.75; N, 6.61. ES-MS m/z 485 [M—CF₃SO₃]⁺. IR (KBr) ν (cm⁻¹) 1624, 1521 (C═O).

### EXAMPLE 94

### AMD8874: Synthesis of [Ru(acac)₂(MeCN)₂(aeae)][CF₃SO₃]

[Bis(acetonitrile)[2-(2-amino-κN-ethylamino-κN′)ethanol]bis[4-(hydroxy-κO)-3-penten-2-onato] ruthenium (III) trifluoromethanesulfonate]

Synthesis of [Ru(acac)₂(MeCN)₂(aeae)][CF₃SO₃

Using General Procedure J

Addition of aeae (85 μL, 0.841 mmol) to a CH₂Cl₂ solution of [Ru(acac)₂(MeCN)₂][CF₃SO₃] (0.391 g, 0.737 mmol) afforded a red/orange solution after 5 hours. The mixture was purified by column chromatography on silica gel (15:1 to 10:1 CH₂Cl₂:MeOH) to give a red/brown solid (0.127 g, 27%).

Anal. Calcd. for C₁₉H₃₂N₄O₈SF₃Ru.1.2CF₃SO₃H.0.8H₂O: C, 29.26; H, 4.23; N, 6.76; S, 8.51. Found: C, 29.25; H, 4.01; N, 6.41; S, 8.40. ES-MS m/z 486 [M—CF₃SO₃]⁺. IR (KBr) ν (cm⁻¹) 2263 (C≡N), 1626, 1550, 1524 (C═O).

### EXAMPLE 95

### AMD8878: Synthesis of [Ru(acac)₂(MeCN)₂(appd)][CF₃SO₃]

[Bis(acetonitrile) [N-(3-aminopropyl)-1,3-propanediamine-κN,κN′]bis[4-(hydroxy-κO)-3-penten-2-onato] ruthenium (III) trifluoromethanesulfonate].

Preparation of [Ru(acac)₂(MeCN)₂(appd)][CF₃SO₃]

Using General Procedure J

Addition of appd (110 μL, 0.774 mmol) to a CH₂Cl₂ solution of [Ru(acac)₂(MeCN)₂][CF₃SO₃] (0.373 g, 0.704 mmol) afforded a red/orange solution after 5 hours. The mixture was purified by column chromatography on silica gel (20:1 to 8:1 CH₂Cl₂:MeOH) to give an orange solid (0.041 g, 9%).

Anal. Calcd. for C₂₁H₃₇N₅O₇SF₃Ru.0.4CF₃SO₃H.0.7CH₂Cl₂: C, 33.98; H, 5.01; N, 8.97; S, 5.75. Found: C, 34.28; H, 4.97; N, 8.33; S, 5.89. ES-MS m/z 513 [M—CF₃SO₃]⁺. IR (KBr) ν (cm⁻¹) 2335, 2289 (C≡N); 1626, 1551 (C═O).

### EXAMPLE 96

### AMD8879: Synthesis of [Ru(acac)₂(MeCN)₂(aepd)][CF₃SO₃]

[Bis(acetonitrile)[N-(2-aminoethyl)-1,3-propanediamine-κN,κN′]bis[4(hydroxy-κO)-3-penten-2-onato] ruthenium (III) trifluoromethanesulfonate]

Preparation of [Ru(acac)₂ (MeCN)₂(aepd)][CF₃SO₃

Following General Procedure J

Addition of aepd (100 μL, 0.782 mmol) to a CH₂Cl₂ solution of [Ru(acac)₂(MeCN)₂][CF₃SO₃] (0.377 g, 0.711 mmol) afforded a red/orange solution after 2 hours. The mixture was purified by column chromatography on silica gel (20:1 to 8:1 CH₂Cl₂:MeOH) to give an orange solid (0.055 g, 12%).

Anal. Calcd. for C₂₀H₃₅N₅O₇SF₃Ru.0.4H₂O: C, 36.68; H, 5.51; N, 10.69; S, 4.90. Found: C, 36.96; H, 5.38; N, 10.33; S, 4.85. ES-MS m/z 499 [M—CF₃SO₃]⁺. IR (KBr) ν (cm⁻¹) 2367, 2334 (C-N), 1624, 1550 (C═O).

### EXAMPLE 97

### AMD8813: Synthesis of [Ru(acac)₂(MeCN)₂(L1)][CF₃SO₃]

[Bis(acetonitile)[N,N-bis[2-(amino-κN)ethyl]-L-isoleucyl-L-prolinato]bis[4-(hydroxy-κO)-3-penten-2-onato] ruthenium (III) trifluoromethanesulfonate]

Synthesis of N,N-bis(2-aminoethyl)-Ile-Pro (L1)

To a solution of nosyl aziridine (0.744 g, 3.26 mmol) in dry THF (20 mL) was added the dipeptide Ile-Pro (0.372 g, 1.63 mmol). The white slurry was stirred for 16 h at 65° C. under N₂ resulting in a clear, yellow solution. The solvent was removed in vacuo to give a yellow oil which was purified by column chromatography on silica gel (3:2 EtOAc:hexanes and then 25:1 CH₂Cl₂:MeOH) to give the desired intermediate as a pale yellow oil (0.377 g, 34 %).

¹H NMR (CDCl₃) δ 0.79 (t, 3H), 0.91 (d, 4H), 1.04 (m, 1H), 1.55 (m, 2H), 1.94 (m, 2H), 2.29 (dm, 1H), 2.79 (m, 2H), 3.35-3.56 (m, 8H), 4.27 (m, 1H), 4.34 (m, 1H), 6.13 (s, 1H), 6.34 (s, 1H), 7.71 (m, 6H), 8.04 (m, 2H); ¹³C NMR (CDCl₃) δ 11.63, 16.13, 24.79, 25.69, 29.26, 38.21, 42.75, 44.20, 47.29, 53.93, 59.69, 64.13, 65.07, 124.74, 125.62, 131.01, 133.47, 133.20, 133.62, 134.03, 134.34, 148.29, 172.31. ES-MS m/z 707 [M+Na]⁺, 685 [M+H]⁺.

To a solution of the oil from above (0.377 g, 0.550 mmol) in dry acetonitrile (15 mL) was added K₂CO₃ (0.761 g, 5.50 mmol) and thiophenol (454 μL, 4.41 mmol). The mixture was stirred for 3.5 h at room temperature under nitrogen, during which time, a bright yellow slurry formed. The mixture was filtered and the solid was washed with acetonitrile. The combined filtrates were evaporated and the residue was purified by column chromatography on neutral alumina using 5:1 CH₂Cl₂:MeOH followed by 7:2:1 CH₂Cl₂:MeOH: NH₄OH to give L1 as a pale yellow oil (0.085 g, 49%). ES-MS m/z 337 [M+Na]⁺, 315 [M+H]⁺.

Preparation of [Ru(acac)₂(MeCN)₂(L1)][CF₃SO₃]

Using General Procedure J

Addition of L1 (0.085 g, 0.271 mmol) to a CH₂Cl₂ solution of [Ru(acac)₂(MeCN)₂][CF₃SO₃] (0.126 g, 0.238 mmol) afforded a red/orange solution after the mixture was heated to reflux for 5 hours. The mixture was purified by column chromatography on silica gel (14:1 to 10:1 CH₂Cl₂:MeOH) to give a deep red solid (0.041 g, 25%).

Anal. Calcd. for C₃₀H₅₀N₆O₁₀SF₃Ru.3.6CH₂Cl₂: C, 35.07; H, 5.01; N, 7.30. Found: C, 35.11; H, 4.90; N, 7.05. ES-MS m/z 696 [M—CF₃SO₃]⁺.

### EXAMPLE 98

### AMD8656: Synthesis of [Ru(acac)₂(S₂CNMe₂)]

[(Dimethylcarbamodithioato-κS,κS′)bis(2,4-pentanedionato-κO,κO′) ruthenium (III)]

General Procedure K

In a schlenk tube, [Ru(μ-diketonato)₂(MeCN)₂][CF₃SO₃] (where β-diketonato=acac or dpac) was dissolved in EtOH:H₂O (20:1) to give a blue or green solution. Addition of a dithiocarbamate salt resulted in an immediate colour change to red/brown. The mixture was stirred at 70° C. for 4-16 h before the solvent was removed under vacuum and the red/brown residue was purified using column chromatography. The dithiocarbamate salts were either purchased from Aldrich (NaS₂CNMe₂.2H₂O) or synthesized according to general procedure F (KS₂CNProK, KS₂CNProOMe, KS₂CNMeIleK).

Preparation of Ru(acac)₂(S₂CNMe₂)

Using General Procedure K

Addition of NaS₂CNMe₂.2H₂O (0.101 g, 0.563 mmol) to a solution of [Ru(acac)₂(MeCN)₂][CF₃SO₃] (0.263 g, 0.496 mmol) in a mixture of ethanol and water gave an immediate colour change from blue to orange. The mixture was stirred at 70° C. for 5 h yielding a red/brown mixture which was purified by column chromatography on silica gel (20:1 CH₂Cl₂:MeOH) to give a dark red solid upon drying in vacuo (0.092 g, 44%).

Anal. Calcd. for C₁₃H₂₀NO₄S₂Ru.0.5EtOH: C, 37.89; H, 5.18; N, 3.19. Found: C, 38.01; H, 4.99; N, 3.26. ES-MS m/z 443 [M+Na]⁺.

### EXAMPLE 99

### AMD8792: Synthesis of [Ru(dpac)₂(S₂CNMe₂)]

[(Dimethylcarbamodithioato-κS,κS′)bis(1,3-diphenyl-1,3-propanedionato-κO,κO′) ruthenium (III)]

Preparation of [Ru(dpac)₂(S₂CNMe₂)]

Using General Procedure K

Addition of NaS₂CNMe₂.2H₂O (0.073 g, 0.409 mmol) to a solution of [Ru(dpac)₂(MeCN)₂][CF₃SO₃] (0.290 g, 0.372 mmol) in a mixture of ethanol and water gave an immediate colour change from green to red/orange. The mixture was stirred at 70° C. for 16 h to give a red/brown mixture which was evaporated and purified by column chromatography on silica gel (5:1 CH₂Cl₂:hexanes) to give a deep red solid (0.025 g, 11%).

Anal. Calcd. for C₃₃H₂₈NO₄S₂Ru.0.3MeCN.0.4hexanes: C, 60.51; H, 4.87; N, 2.55; S, 8.97. Found: C, 60.25; H, 4.90; N, 2.38; S, 8.50. ES-MS m/z 650 [M+Na]⁺. IR (KBr) ν (cm⁻¹) 1514 (C═O).

### EXAMPLE 100

### AMD8822: Synthesis of [Ru(acac)₂(S₂CNProOMe)]

[(1-carboxymethyl)-1,4-butanediylcarbamodithioato-κS,κS′]bis(2,4-pentanedionato-κO,κO′) ruthenium (III).

Preparation of [Ru(acac)₂(S₂CNProOMe)]

Using General Procedure K

Addition of KS₂CNProOMe (0.548 g, 2.24 mmol) to solution of [Ru(acac)₂(MeCN)₂][CF₃SO₃] (1.06 g, 2.00 mmol) in a mixture of ethanol and water gave an immediate colour change from blue to orange. The mixture was stirred at 70° C. for 4 h to give a red/orange mixture which was evaporated and the residue purified by column chromatography on silica gel (50:1 CH₂Cl₂:MeOH) to give a deep red solid (0.147 g, 13%).

Anal. Calcd. for C₁₇H₂₄NO₆S₂Ru: C, 40.55; H, 4.80; N, 2.78; S, 12.73. Found: C, 40.68; H, 4.82; N, 2.76; S, 12.60. ES-MS m/z 527 [M+Na]⁺, 505 [M+H]⁺. IR (KBr) ν (cm⁻¹) 1746 (CO₂Me), 1549 (C═O).

### EXAMPLE 101

### AMD8823 and AMD8826: Synthesis of Ru(dpac)₂(S₂CNProOMe) and Ru(dpac)₂(Pro).

[(1-carboxymethyl)-1,4-butanediylcarbamodithioato-κS,κS′]bis(1,3-diphenyl-1,3-propanedionato-κO,κO′) ruthenium (III) and [L-prolinato(1−)-κN,κO]bis(1,3-diphenyl-1,3-propanedionato-κO,κO′) ruthenium (III)

Synthesis of Ru(dpac)₂(S₂CNProOMe) and Ru(dpac)₂(Pro)

Using General Procedure K

Reaction of KS₂CNProOMe (0.382 g, 2.24 mmol) with [Ru(dpac)₂(MeCN)₂][CF₃SO₃] (0.947 g, 1.22 mmol) in ethanol/water solution followed by purification of the reaction mixture by column chromatography on silica gel (50:1 CH₂Cl₂:MeOH) gave two products. A red solid whose characterisation data was consistent with [Ru(dpac)₂(S₂CNProOMe)] (0.065 g, 5%).

Anal. Calcd. for C₃₇H₃₂NO₆S₂Ru.0.3dpac.1.0OEtOH: C, 60.41; H, 4.81; N, 1.62; S, 7.41. Found: C, 60.48; H, 4.91; N, 1.80; S, 7.64. ES-MS m/z 752 [M+H]⁺. IR (KBr) ν (cm⁻¹) 1746 (CO₂Me); 1587 (C═O). An orange/brown solid whose characterisation data were consistent with [Ru(dpac)₂(Pro)] (0.095 g, 18%).

Anal. Calcd. for C₃₅H₂₉NO₆Ru: C, 63.63; H, 4.42; N, 2.12. Found: C, 63.45; H, 4.43; N, 2.24. ES-MS m/z 661 [M+H]⁺. IR (KBr) ν (cm⁻¹) 1667 (CO₂⁻), 1586 (C═O).

### EXAMPLE 102

### AMD8736: Synthesis of [Ru(acac)₂(S₂CNProK)]

[Potassium[(1-carboxy)-1,4-butanediylcarbamodithioato-κS,κS′]bis(2,4-pentanedionato-κO,κO′) ruthenium (III)]

Preparation of [Ru(acac)₂(S₂CNProK)]

Using General Procedure K

Reaction of KS₂CNProK (0.422 g, 1.58 mmol) with [Ru(acac)₂(MeCN)₂][CF₃SO₃] (0.756 g, 1.42 mmol) gave a dark blue slurry. The mixture was stirred at reflux for 1 h giving a red/black mixture, which was evaporated to dryness. Sonication of the residue with CH₂Cl₂ gave a black solid, which was removed via filtration. The filtrate was purified by column chromatography on silica gel (20:1 to 12:1 CH₂Cl₂:MeOH) to give a deep red solid (0.105 g, 15%).

Anal. Calcd. for C₁₆H₂₁NO₆S₂RuK.2.1H₂O.0.2KCF₃SO₃: C, 32.26; H, 4.21; N, 2.32; S, 11.69. Found: C, 32.43; H, 4.25; N, 2.25; S, 11.66. ES-MS m/z 490 [M+H]⁺. IR (KBr) ν (cm⁻¹) 1558 (C═O).

### EXAMPLE 103

### AMD8791: Synthesis of [Ru(acac)₂(NMeIle)]

[N-methyl-L-isoleucinato(1-)-κN,κO]bis(2,4-pentanedionato-κO,κO′) ruthenium (III)

Preparation of [Ru(acac)₂(NMeIle)]

Using General Procedure K

Reaction of KS₂CNMeIleK (0.269 g, 0.903 mmol) with [Ru(acac)₂(MeCN)₂][CF₃SO₃] (0.445 g, 0.839 mmol) in a mixture of ethanol and water gave an immediate colour change from blue to orange/brown. The mixture was stirred at 70° C. for 7 h giving a redibrown solution. The volume of the reaction mixture was reduced to ˜3 mL and Et₂O was added to give a brown precipitate, which was separated by filtration. The filtrate was purified by column chromatography on silica gel (20:1 CH₂Cl₂:MeOH) to give an orange/brown solid (0.050 g, 12%).

Anal. Calcd. for C₁₇H₂₇NO₆Ru.0.2C₄H₁₀O: C, 46.75; H, 6.39; N, 3.06. Found: C, 47.03; H, 6.16; N, 3.28. ES-MS m/z 465 [M+Na]⁺, 443 [M+H]⁺. IR (KBr) ν (cm⁻¹) 1670, 1560 (C═O).

### EXAMPLE 104

### AMD8795: Synthesis of [Ru(acac)₂(NMeIle)]₂

Bis[μ-[N-methyl-L-isoleucinato(1−)-κN:κO]]tetrakis(2,4-pentanedionato-κO,κO′) diruthenium (III).

Preparation of [Ru(acac)₂(NMeIle)]₂

[Ru(acac)₂(MeCN)₂][CF₃SO₃ (0.270 g, 0.508 mmol) was dissolved in EtOH (6 mL) to give a dark blue solution. NMeIle (0.084 g, 0.581 mmol) was added and the mixture was stirred at 75° C. for 16 h to give an orange solution. The solvent was removed under reduced pressure and the orange residue was purified by column chromatography on silica gel (20:1 CH₂Cl₂:MeOH) to give an orange solid (0.150 g, 67%).

Anal. Calcd. for C₃₄H₅₆N₂O₁₂Ru₂.0.3C₆H₁₄: C, 47.11; H, 6.65; N, 3.07. Found: C, 47.21; H, 6.62; N, 3.08. ES-MS m/z 911 [M+Na]⁺. IR (KBr) ν (cm⁻¹) 1649, 1552 (C═O).

### EXAMPLE 105

### AMD8845: Synthesis of [Ru(dpac)₂(Pro)]₂

[Bis[μ-[L-prolinato(1−)-κN:κO]]tetrakis(1,3-diphenyl-1,3-propanedionato-κO,κO′) diruthenium (III)]

Preparation of [Ru(dpac)₂(Pro)]₂

[Ru(dpac)₂(MeCN)₂][CF₃SO₃] (0.493 g, 0.633 mmol) was dissolved in EtOH (8 mL) to give a dark green solution. (L)-Proline (0.078 g, 0.677 mmol) was added and the mixture was stirred at 75° C. for 16 h to give a brown/orange solution. The solvent was removed under reduced pressure and the brown residue was purified by column chromatography on silica gel (50:1 CH₂Cl₂:MeOH) to give an orange/brown solid (0.035 g, 8%).

Anal. Calcd. for C₇₀H₆₀N₂O₁₂Ru₂.0.4CH₂Cl₂: C, 62.43; H, 4.50; N, 2.06. Found: C, 62.44; H, 4.53; N, 1.98. ES-MS m/z 1345 [M+Na]⁺. IR (KBr) ν (cm⁻¹) 1666, 1522 (C═O).

### EXAMPLE 106

### AMD8856: Synthesis of Ru(acac)₂(2-pyridine thiolato)(2-pyridinethione)

[bis(2,4-pentanedionato-κO,κO′)[2(1H)-pyridinethionato-κS²][2(1H)-pyridinethione-κS²] ruthenium (III)]

Preparation of Ru(acac)₂(2MP)₂

[Ru(acac)₂(MeCN)₂][CF₃SO₃] (0.399 g, 0.751 mmol) was dissolved in EtOH (10 mL) to give a dark blue solution. 2-Mercaptopyridine (0.340 g, 3.06 mmol) was added and the mixture was stirred and heated at 75° C. for 5 h to give a red/purple solution. The solvent was removed under reduced pressure and the purple residue was purified via preparative TLC on silica gel (20:1 CH₂Cl₂:MeOH) to give a purple solid (0.057 g, 14%).

Anal. Calcd. for C₂₀H₂₃N₂O₄S₂Ru: C, 46.14; H, 4.45; N, 5.38; S, 12.32. Found: C, 46.15; H, 4.48; N, 5.42; S, 12.23. ES-MS m/z 522 [M+H]⁺. IR (KBr) ν (cm⁻¹) 1545 (C═O), 1120 (C═S).

### EXAMPLE 107

### AMD8857: Synthesis of Ru(acac)₂(η²-2-pyridinethiolato)

[bis(2,4-pentanedionato-κO,κO′)[2(1H)-pyridinethionato-κN,κS²] ruthenium (III)]

Preparation of [Ru(acac)₂(2MP)]

[Ru(acac)₂(MeCN)₂][CF₃SO₃] (0.292 g, 0.550 mmol) was dissolved in EtOH (10 mL) to give a dark blue solution. 2-Mercaptopyridine (0.065 g, 0.588 mmol) and KOH (0.036 g, 0.645 mmol) were added to give an instantaneous orange solution. The mixture was stirred at 80° C. for 4 h to give a turquoise solution. The solvent was removed under reduced pressure and the blue residue was purified by column chromatography on silica gel (25:1 CH₂Cl₂:MeOH). A turquoise blue band was isolated which was further purified via preparative TLC to afford a blue solid (0.089 g, 40%).

Anal. Calcd. for C₁₅H₁₈NO₄SRu.0.3C₃H₆O: C, 44.74; H, 4.68; N, 3.28; S, 7.51. Found: C, 44.70; H, 4.55; N, 3.37; S, 7.51. ES-MS m/z 433 [M+Na]⁺, 411 [M+H]⁺. IR (KBr) ν (cm⁻¹) 1545 (C═O).

### EXAMPLE 108

### AMD8865: Synthesis of [Ru(acac)₂(4ImP)₂][CF₃SO₃]

[bis(2,4-pentanedionato-κO,κO′)bis[4-(1H-imidazol-1-yl-κN³)phenol] ruthenium (III) trifluoromethanesulfonate]

Preparation of [Ru(acac)₂(4ImP)₂][CF₃SO₃]

[Ru(acac)₂(MeCN)₂][CF₃SO₃] (0.405 g, 0.550 mmol) was dissolved in EtOH (10 mL) to give a dark blue solution. 4-(Imidazol-1-yl)phenol (4ImP) (0.538 g, 3.36 mmol) was added and the mixture was stirred at 80° C. for 21 h to give a deep red solution. The solvent was removed under reduced pressure and the residue was purified by column chromatography on silica gel (20:1 CH₂Cl₂:MeOH) to give a red crystalline solid (0.203 g, 34%).

Anal. Calcd. for C₂₉H₃₀N₄O₉SF₃Ru: C, 45.31; H, 3.93; N, 7.29; S, 4.17. Found: C, 45.44; H, 4.11; N, 7.00; S, 3.88. ES-MS m/z 620 [M—CF₃SO₃]⁺. IR (KBr) ν (cm⁻¹) 1524 (C═O).

### EXAMPLE 109

### AMD8873 and AMD8877: Synthesis of [Ru(dpac)₂(4ImP)(MeCN)][CF₃SO₃].EtOH and [Ru(dpac)₂(4ImP)₂][CF₃SO₃]

[(Acetonitrile)bis(1,3-diphenyl-1,3-propanedionato-κO,κO′)[4-(1H-imidazol-1-yl-κN³)phenol] ruthenium (III) trifluoromethanesulfonate] and [bis(1,3-diphenyl-1,3-propanedionato-κO,κO′)bis[4-(1H-imidazol-1-yl-N³)phenol] ruthenium (III) trifluoromethanesulfonate]

[Ru(dpac)₂(MeCN)₂][CF₃SO₃] (0.305 g, 0.341 mmol) was dissolved in EtOH (10 mL) to give a dark green solution. 4-(Imidazol-1-yl)phenol (0.327 g, 2.04 mmol) was added and the mixture was stirred at 80° C. for 24 h to give a brown solution. The solvent was removed under reduced pressure and the brown residue was purified by column chromatography on silica gel (20:1 CH₂Cl₂:MeOH) to give two products: [Ru(dpac)₂(4ImP)₂][CF₃SO₃] as a brown solid (0.080 g, 25%).

Anal. Calcd. for C₄₄H₃₉N₃O₉SF₃Ru: C, 55.99; H, 4.16; N, 4.45; S, 3.40. Found: C, 56.18; H, 4.25; N, 4.46; S, 3.16. ES-MS m/z 795 [M—CF₃SO₃]⁺. IR (KBr) ν (cm⁻¹) 2361 (C≡N), 1522 (C═O); and [Ru(dpac)₂(4ImP)(MeCN)][CF₃SO₃]-EtOH as a brown solid (0.085 g, 24%).

Anal. Calcd. for C₄₉H₃₈N₄O₉SF₃Ru.3.4C₉H₈N₂O: C, 61.22; H, 4.21; N, 9.69; S, 2.05. Found: C, 61.51; H, 4.44; N, 9.42; S, 1.87. ES-MS m/z 868 [M—CF₃SO₃]⁺. IR (KBr) ν (cm⁻¹) 1522 (C═O).

### EXAMPLE 110

### AMD8866: Synthesis of [Ru(acac)₂(ImProOMe)₂][CF₃SO₃]

[Bis[methyl-1-[(1H-imidazol-1-yl-κN³)acetyl]-L-prolinate]bis(2,4-pentanedionato-κO,κO′) ruthenium (III) trifluoromethanesulfonate]

Synthesis of the ligand: ImProOMe

N-(2-chloro)acetamido-(L)-proline methyl ester

Chloroacetic acid (0.674 g, 7.13 mmol) was dissolved in THF (40 mL) at 0° C. under nitrogen. N-Methylmorpholine (784 μL, 7.18 mmol) was then added and the colourless mixture was stirred for 10 minutes iso-butylchloroformate (1.01 mL, 7.84 mmol) was added and the mixture was stirred for 30 min during which a white slurry was formed. The ice-bath was removed, and (L)-proline methyl ester (0.600 g, 4.65 mmol) and N-methylmorpholine (550 μL, 5.04 mmol) were added. The reaction slurry was stirred at room temperature for 5.5 h and the resulting white precipitate was filtered off and washed with THF (3×5 mL). The combined filtrates were evaporated to dryness and the residue was purified by column chromatography on silica gel (22:1 CH₂Cl₂:MeOH) to give the title compound as a pale yellow oil (0.422 g, 44%). ES-MS m/z 206 [M+H]⁺.

¹H NMR (CDCl₃) δ 1.96 (m, 2H), 2.14 (m, 2H), 3.56 (m, 2H), 3.63 (s, 3H), 3.96 (d, 2H, J=3.3 Hz), 4.42 (dd, 1H, J=8.5 Hz); ¹³C NMR (CDCl₃) δ 25.2, 29.5, 42.3, 47.4, 52.7, 59.7, 165.2, 172.5.

Preparation of ImProOMe

N-(2-chloro)acetamido-(L)-proline methyl ester (0.422 g, 2.05 mmol) was added to a suspension of sodium imidazolate (0.281 g, 3.12 mmol) in DMF (5 mL) at room temperature and the mixture was heated to 75° C. for a further 16 hours. The reaction mixture was evaporated and the residue was purified by column chromatography on silica gel (20:1 CH₂Cl₂:MeOH) to give white crystalline solid (0.244 g, 50%). ES-MS m/z 238 [M+H]⁺.

¹H NMR (CDCl₃) δ 1.83-2.11 (m, 4H), 3.34-3.46 (m, 2H), 3.54 (s, 3H), 4.33 (dd, 1H, J=8.4 Hz), 3.61 (s, 2H), 6.82 (s, 1H), 6.87 (s, 1H), 7.34 (s, 1H); ¹³C NMR (CDCl₃) δ 25.1, 29.2, 46.6, 48.8, 53.3, 59.5, 120.7, 129.3, 138.4, 165.5, 172.5.

Preparation of [Ru(acac)₂(ImProOMe)₂][CF₃SO₃]

[Ru(acac)₂(MeCN)₂][CF₃SO₃] (0.275 g, 0.518 mmol) was dissolved in EtOH (10 mL) to give a dark blue solution. ImProOMe (0.244 g, 1.08 mmol) was added and the mixture was stirred and heated at 80° C. for 20 h to give a red/purple solution. The solvent was removed under reduced pressure and the residue was purified by column chromatography on silica gel (20:1 CH₂Cl₂:MeOH) to give a red solid (0.127 g, 32%).

Anal. Calcd. for C₃₃H₄₄N₆O₁₃SF₃Ru: C, 42.95; H, 4.81; N, 9.11; S, 3.47. Found: C, 43.06; H, 4.94; N, 8.83; S, 3.27. ES-MS m/z 774 [M—CF₃SO₃]⁺. IR (KBr) ν (cm⁻¹) 1670, 1522 (C═O).

### EXAMPLE 111

### AMD8891: Synthesis of [Ru(acac)₂(histamine)(MeCN)] [CF₃SO₃]

[(Acetonitrile)(4-ethylamino-1H-imidazol-κN³)bis(2,4-pentanedionato-κO,κO′) ruthenium (III)]

Preparation of [Ru(acac)₂(histamine)(MeCN)][CF₃SO₃]

[Ru(acac)₂(MeCN)₂][CF₃SO₃] (0.338 g, 0.638 mmol) was dissolved in EtOH (10 mL) to give a dark blue solution. Histamine (0.083 g, 0.744 mmol) was added and the mixture was stirred at 80° C. for 1 h and then at room temperature for 18 h to give a red/brown solution. The solvent was removed under reduced pressure and the brown residue was purified by column chromatography on silica gel (20:1 CH₂Cl₂:MeOH) to give an orange solid (0.066 g, 17%).

Anal. Calcd. for C₁₈H₂₆N₄O₇SF₃Ru.0.9C₃H₆O: C, 38.09; H, 4.85; N, 8.58; S, 4.91. Found: C, 38.15; H, 4.61; N, 8.41; S, 4.70. ES-MS m/z 452 [M—CF₃SO₃]⁺. IR (KBr) ν (cm⁻¹) 2291 (C≡N), 1670, 1547 (C═O).

### EXAMPLE 112

### AMD8903: Preparation of [Ru(edtmp)].3H₂O

A mixture of K₂[RuCl₅(H₂O)] (0.35 g) and ethylenediaminetetraphosphonic acid, edtmp (0.40 g) in water (15 mL) was heated to reflux for one hour. The dark solution was allowed to stand for 2 days then evaporated to approximately 3 mL. Methanol (˜15 mL) was added resulting in the formation of green precipitate. The solid was collected by filtration and methanol was added to the filtrate to precipitate a yellow solid. The yellow solid was also collected by filtration, washed with ether and dried in vacuo to give the title compound (60 mg, 11%).

Anal. Calcd. for C₆H₂₃N₂P₄O₁₅Ru: C, 12.24; H, 3.95; N, 4.76. Found: C, 11.82; H, 3.43; N, 4.43.

### EXAMPLE 113

### AMD6245: Preparation of [Ru(Hedta)]H₂O

K[Ru(Hedta)Cl].2H₂O (16.0 g, 0.032 mmol) was heated to reflux in de-ionized water (750 mL) for 2 hours. The volume of the solution was reduced to one half the original volume and the solution was seeded with approximately 2-3 mg Ru(Hedta)(OH₂). Upon cooling a precipitate formed which was removed by filtration and washed with ice-cold water, ethanol and diethyl ether. The product was dried in vacuo at 40° C. overnight (10.0 g, 77%).

Anal. Calcd. for C₁₀H₁₅N₂O₉Ru: C, 29.42; H, 3.70; N, 6.86; Cl, 0.0. Found: C, 29.34; H, 3.66; N, 6.92; Cl, 0.0. IR (CsI) ν (cm⁻¹) 3148 (OH); 1741 (CO₂H); 1651 (CO₂). (Mukaida et al, Nippon Kagaku Zasshi, 86, 589 (1965))

### EXAMPLE 114.

Results on the inhibition of tumour growth by AMD6245 and AMD6221

NO is important in controlling tumour growth and vascularisation (Thomsen et al., Cancer and Metastasis Rev. 17 107-118, (1998); Jenkins et al., Proc. Natl. Acad. Sci. USA, 92,4392-4396, (1995); Edwards et al., J. Surg. Res., 63, 49-52, (1996)). Nitric oxide synthases have been shown to be expressed in numerous human and rodent cancers including human gynecological cancers (Thomsen et al., Cancer Res., 54, 1352-1354, (1994), Thomsen et al., Biochem. Pharmacol., 56, 1365-1370, (1998)) and the stroma of human breast cancers (Thomsen et al., Br. J. Cancer, 72, 41-44, (1995)), human lung cancer (Ambs et al., Br. J. Cancer, 78, 233-239, (1998)), human colon cancer (Ambs et al., Cancer Res., 58, 334-341, (1998)), and rat colon tumours (Takahashi et al., Cancer Res., 57, 1233-1237, (1997)). Nitric oxide is an active mediator of angiogenesis (growth of new blood vessels) (Fukumura et al., Cancer and Metastasis Rev., 17, 77-89, (1998); Ziche et al., J. Clin. Invest., 99, 2625-2634, (1997); Gallo et al., J. Natl. Cancer Inst., 90, 587-596(1998)). The establishment of an adequate blood supply is essential to the growth of solid tumours. In addition nitric oxide has been shown to be important for the maintaining the vasodilatory tone of tumours (Tozer et al., Cancer Res, 57, 948-955, (1997)), regulating tumour blood flow (Tozer et al., Cancer Res, 57, 948-955, (1997), Doi et al., Cancer, 77, 1598-1604, (1996)) and tumour oxygenation and energy status (Wood et al., Biochem. Biphys. Res. Commun., 192, 505-510, (1993)). The angiogenic process is intimately linked with metastasis of solid tumours. Nitric oxide increased vascular permeability in tumour bearing mice, (Doi et al., Cancer, 77, 1598-1604, (1996); Maeda et al., Jpn. J. Cancer Res., 85, 331-334, (1994); Wu et al, Cancer Res., 58, 159-165, (1998)) a prerequisite for metastasis. The inhibition of NO synthesis by a NOS inhibitor has been shown to inhibit an increase in metastases and tumour size associated with increased NO production in the EMT-6 murine breast tumour (Edwards et al., J. Surg. Res., 63, 49-52, (1996)). Administration of a NOS inhibitors has been shown to inhibit the growth of experimental tumours in vivo (Kennovin et al., in Biology of Nitric Oxide, Vol. 4, (S. Moncada, M. Feelisch, R. Busse, and A. E. Higgs, eds.), Portland Press, London, 1994, pp. 473-479), Thomsen et al., Cancer Res., 57, 3300-3304, (1997)).

The effect of AMD6245 (Example 113) and AMD6221 (Example 8) on tumour growth was assessed using the rat P22 carcinosarcoma grown in BD-IX rats (Kennovin et al., in Biology of Nitric Oxide, Vol. 4, (S. Moncada, M. Feelisch, R. Busse, and A. E. Higgs, eds.), Portland Press, London, 1994, pp. 473-479). The tumour was implanted subcutaneously on the dorsal surface of male BD-IX rats on Day 0. Tumour growth was measured daily using calipers and tumour volume calculated from the equation Volume=(X².Y²)π/6 where X=the short tumour axis and Y=the long tumour axis. Tumours were measurable by Day 10. AMD6245 and AMD6221 were administered daily by intraperitoneal injection at a dose of 50 mg/kg from Day 10-Day 28. Tumour vascularisation (Microvascular Density or MVD) was measured by Chalkley point counting after immunostaining with anti-CD31 antibody (Vermeulen et al., Eur. J. Cancer, 32A, 2474-2484, (1996)). Nitrite/nitrate was measured by the Griess assay (see Table 4). These anions are the stable end products of NO in solution. Nitrate was first reduced to nitrite by nitrite reductase. The sum of nitrite and nitrate gives the total NO production.

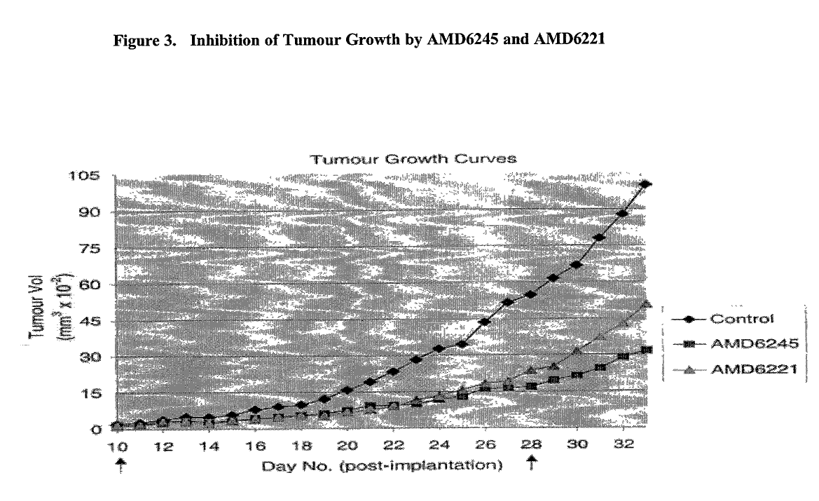

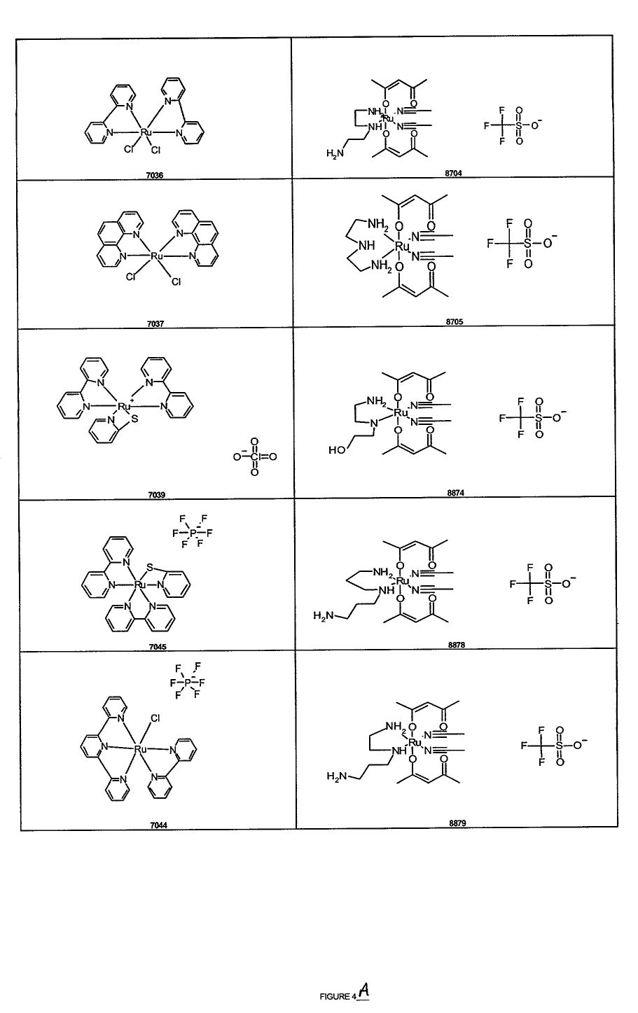

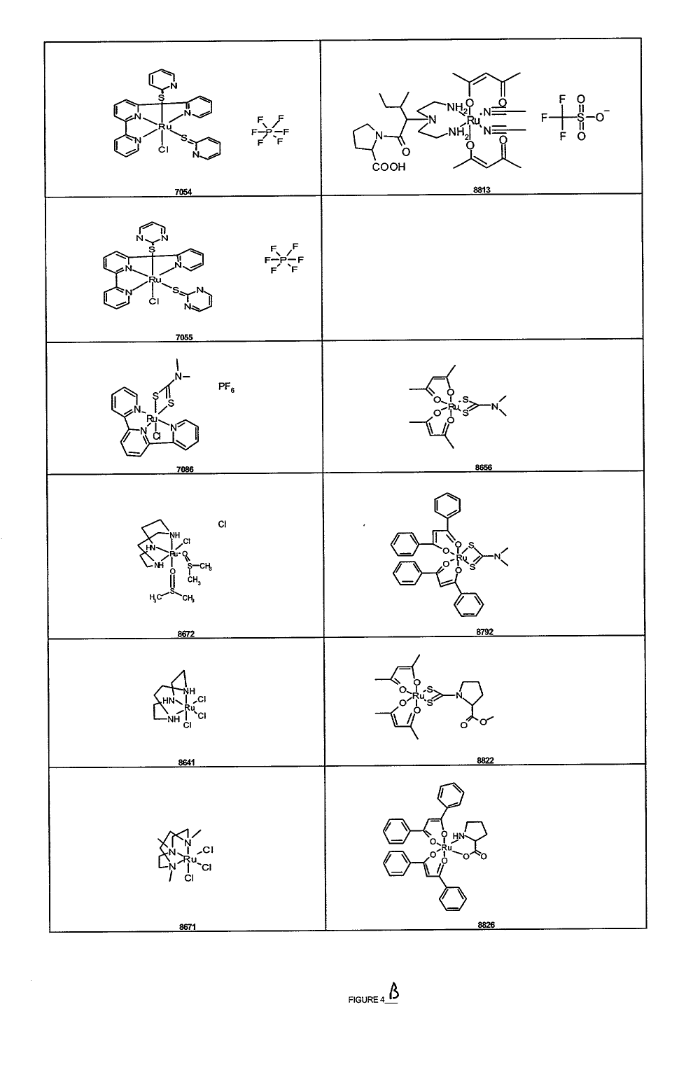

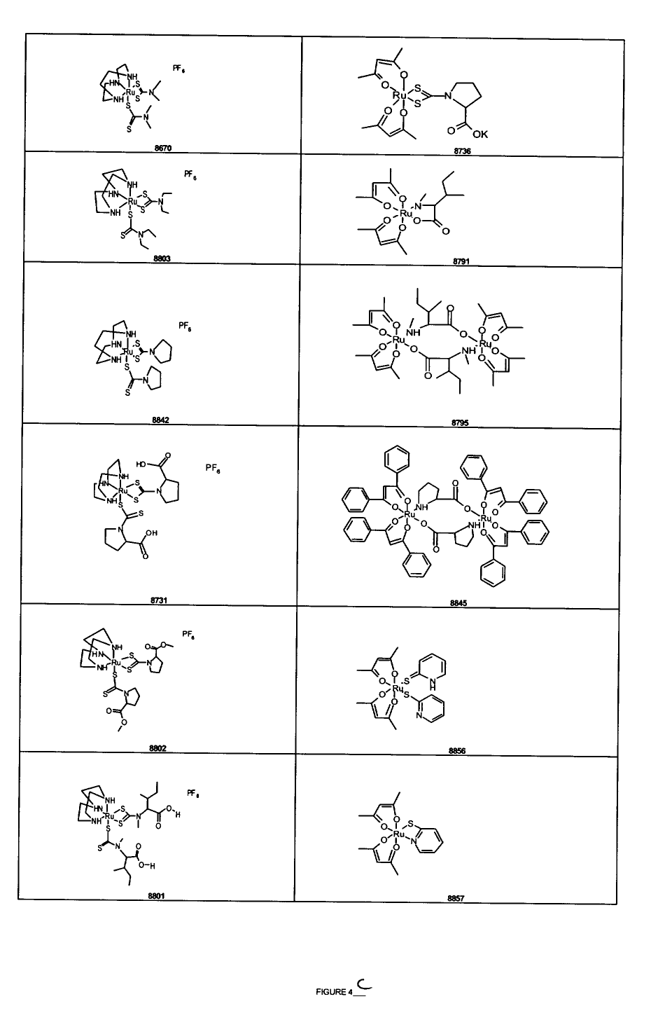

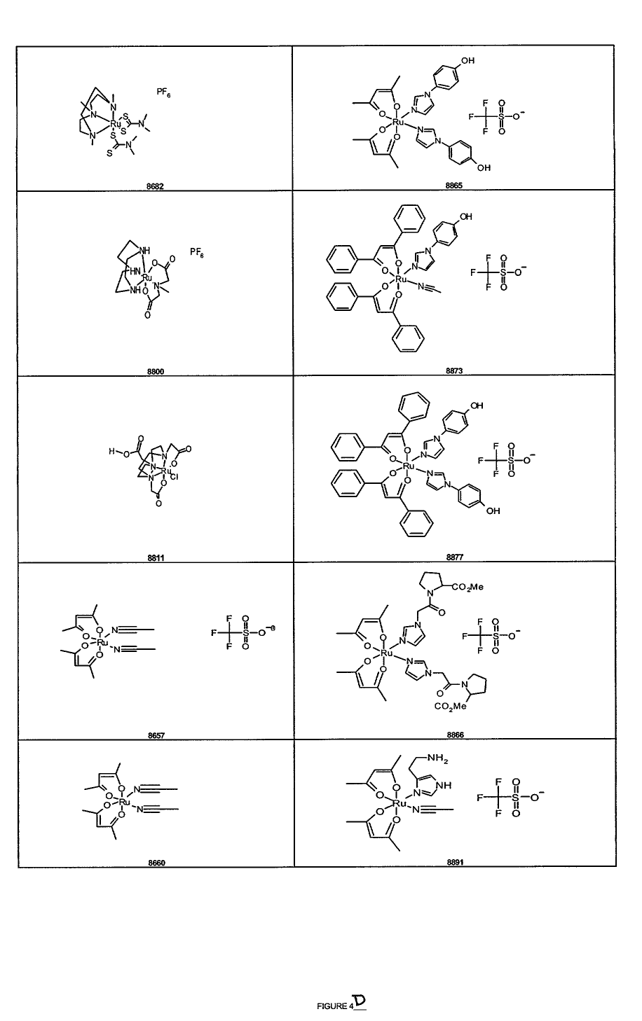

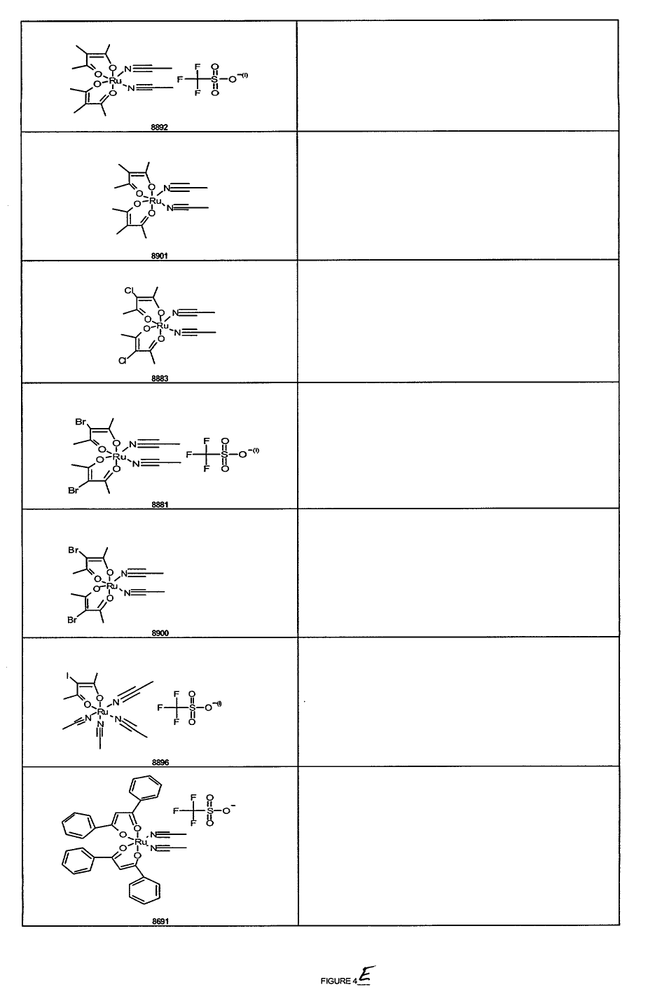

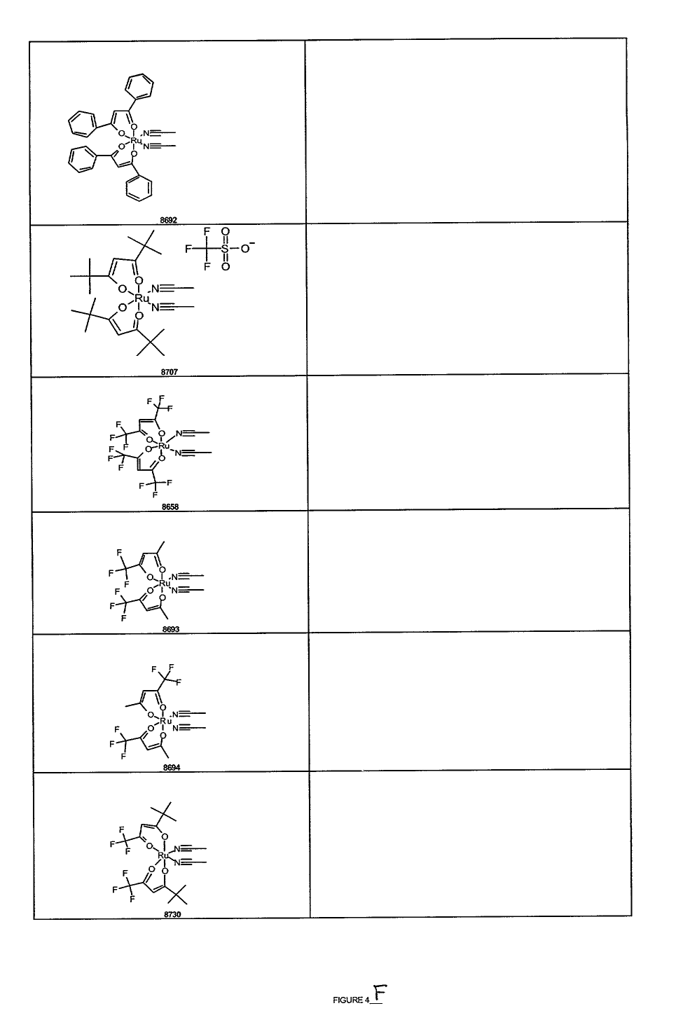

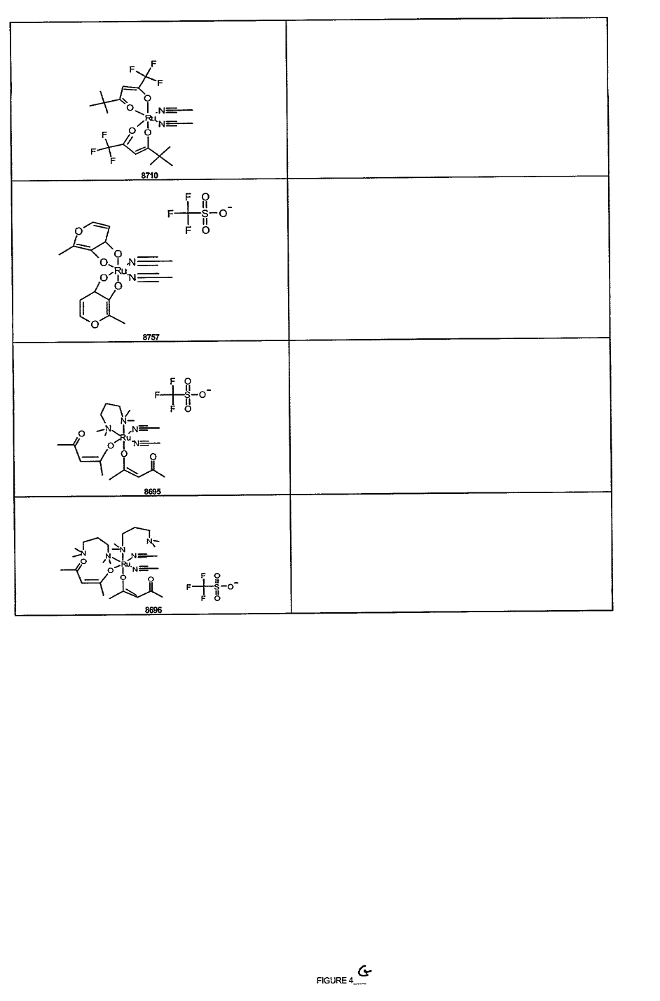

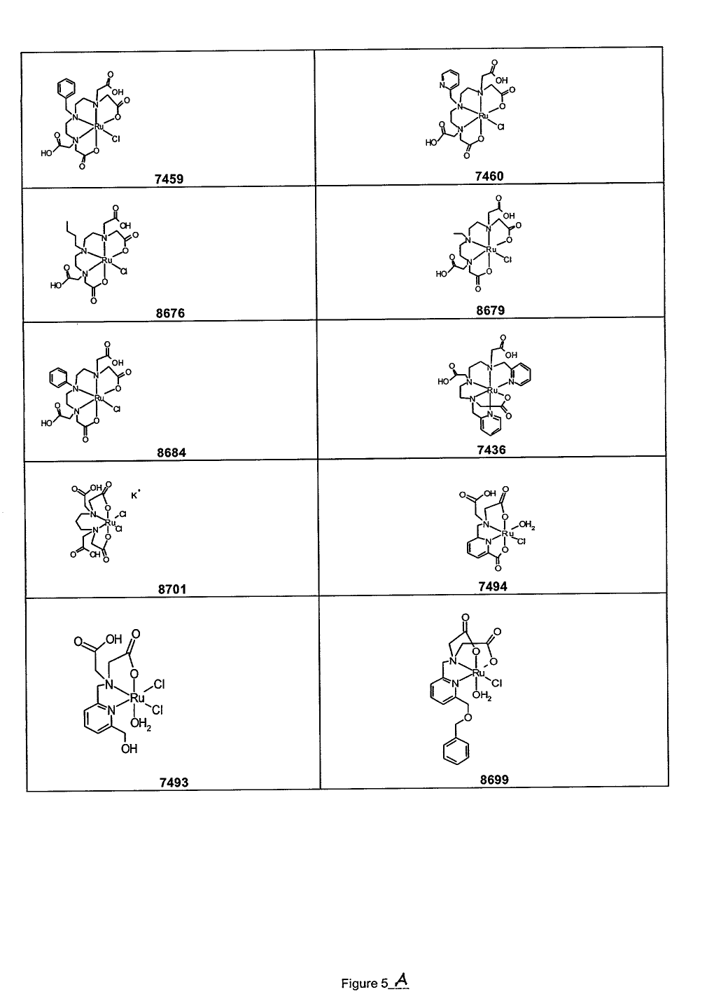

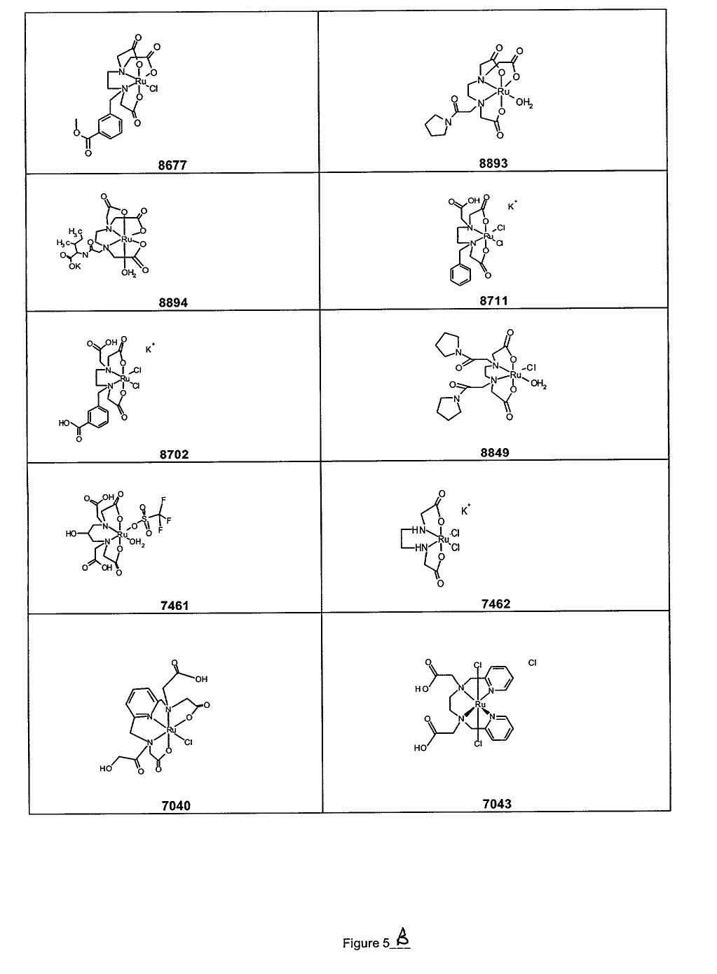

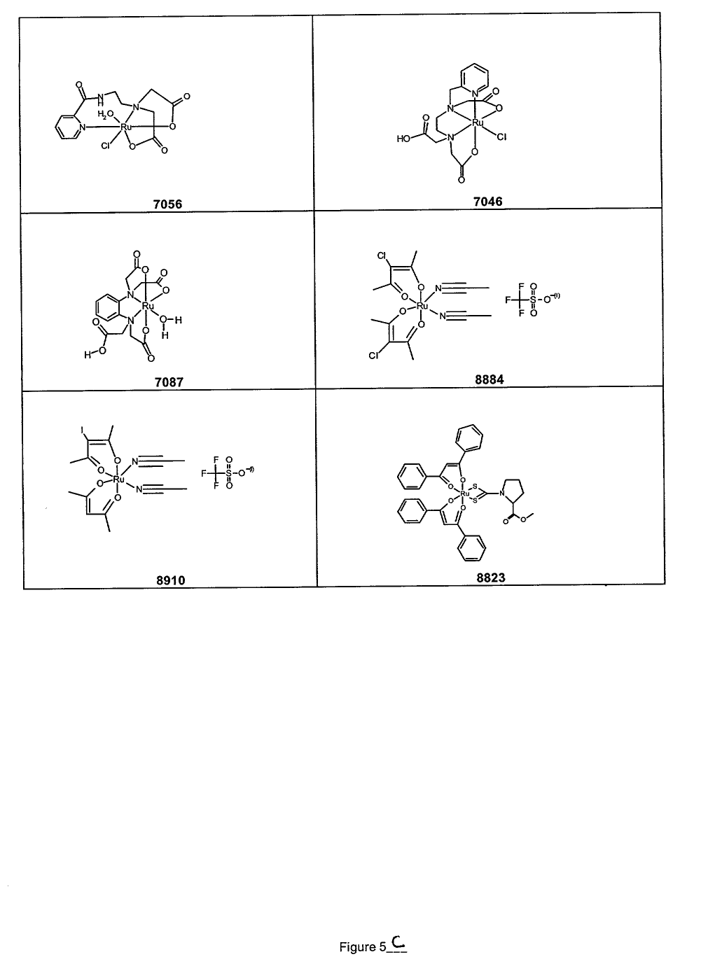

AMD6245 and AMD6221 inhibited the growth of the P22 carcinosarcoma (FIG. 3). Tumour vascularisation (MVD) was lower in tumours from AMD6245 treated animals (Mean Chalkey score=3.0) and AMD6221 treated animals (Mean Chalkey score=5.3) compared with untreated, control tumours (Mean Chalkey score=13.0). Nitrite/nitrate levels at Day 28 were lower in AMD6245 treated animals (3.88 μmoles/litre plasma) and AMD6221 treated animals (5.09 μmoles/litre plasma), compared with untreated, control animals (7.75 μmoles/litre plasma). Therefore, AMD6245 and AMD6221 inhibited tumour growth. This was associated with a decrease in tumour blood supply and a decrease in plasma NO levels.

TABLE 4
Results are presented as net decrease in
nitrite in the stimulated in vitro RAW264 cell
culture supernatant as measured by the Griess assay.

| | Δ Nitrite | | | Δ Nitrite | |
|---|---|---|---|---|---|
| AMD # | (μM) | Concⁿ (μM) | AMD # | (μM) | Concⁿ (μM) |
| 7459 | 19.3 | 100 | 8884 | | |
| 7460 | 21.4 | 100 | 8881 | | |
| 8676 | 24.9 | 100 | 8900 | | |
| 8679 | 38.5 | 100 | 8910 | | |
| 8684 | | | 8896 | 34.5 | 50 |
| 7436 | 4.9 | 100 | 8691 | 25.3 | 50 |
| 8701 | 5.1 | 50 | 8692 | | |
| 7494 | 12.2 | 100 | 8707 | | |
| 7493 | 13 | 100 | 8658 | 5.1 | 25 |
| 8699 | 14.9 | 50 | 8693 | | |
| 8677 | 3.6 | 50 | 8694 | 18.8 | 25 |
| 8893 | 6.6 | 25 | 8730 | | |
| 8894 | | | 8710 | | |
| 8711 | 4.4 | 50 | 8757 | 38.1 | 100 |
| 8702 | 5.2 | 100 | 8695 | | |
| 8849 | 8.8 | 50 | 8696 | 26.4 | 100 |
| 7461 | 12.7 | 100 | 8704 | | |
| 7462 | 7.8 | 100 | 8705 | 37.4 | 100 |
| 8672 | 15.2 | 100 | 8874 | 26.3 | 25 |
| 8641 | | | 8878 | | |
| 8671 | 3.5 | 100 | 8879 | | |
| 8670 | 43.4 | 50 | 8813 | | |
| 8803 | | | 8656 | | |
| 8842 | | | 8792 | | |
| 8731 | 24 | 50 | 8822 | | |
| 8802 | 28.9 | 25 | 8823 | | |
| 8801 | 19 | 25 | 8826 | | |
| 8682 | 23.9 | 50 | 8736 | 36.5 | 100 |
| 8800 | 18.6 | 50 | 8791 | | |
| 8811 | 9.3 | 50 | 8795 | 39.1 | 25 |
| 7044 | 4.9 | 100 | 8845 | | |
| 7054 | 15.9 | 100 | 8856 | | |
| 7055 | 37.7 | 50 | 8857 | | |
| 7086 | 14.8 | 25 | 8865 | 47.2 | 50 |
| 7036 | 7.3 | 100 | 8873 | | |
| 7037 | 4.8 | 100 | 8877 | 15.3 | 25 |
| 7039 | 18.7 | 50 | 8866 | 15.3 | 25 |
| 7045 | 24 | 50 | 8891 | | |
| 8657 | 39.4 | 50 | 6245 | 12.2 | 100 |
| 8660 | 40.4 | 100 | | | |
| 8892 | | | | | |
| 8901 | | | | | |
| 8883 | | | | | |

Typical result for AMD6221 is 37.6 μM at 100 μM, 250 μM L-NMMA gives similar results to 100 μM AMD6221. All compounds were tested at 100 μM unless otherwise stated. The lower concentrations were used because of toxicity at 100 μM.

TABLE 5
COMPOUND NAMES SUMMARY

| AMD | Dihydrogen chloro [[2,6-(pyridinyl-κN)methyl]bis[N- |
|---|---|
| 7040 | (carboxymethyl)glycinato-κN,κO]] ruthenium (III) |
| AMD | Dihydrogen dichloro[[N,N′-1,2-ethanediyl]bis[(2-pyridinyl- |
| 7043 | κN)methylglycinato-κN] ruthenium (III) chloride |
| AMD | Aquachloro[[N-2-[(2-pyridinyl-κN)oxo-methyl)aminoethyl] |
| 7056 | [((2-carboxy-κO)methyl)glycinato-κN,κO]] ruthenium (III) |
| AMD | Hydrogen chloro[N-[bis((2-(carboxy-κO)methyl)imino-κN)ethyl]- |
| 7046 | (2-pyridinyl-κN)methylglycinato-κN] ruthenium (III) |
| AMD | Hydrogen aqua[N-bis((2-carboxy-κO)methyl)imino-κN]- |
| 7087 | 1,2-phendiyl(2-(carboxy-κO)methyl)glycinato-κN] |
| | ruthenium (III) |
| AMD | Dihydrogen chloro[[N,N′-[[(phenylmethyl)imino-κN]-2,1- |
| 7459 | ethanediyl]bis[N-(carboxymethyl)glycinato-κN,κO]] |
| | ruthenium (III) |
| AMD | Dihydrogen chloro[[N,N′-[[(2-pyridinylmethyl)imino-κN]di-2,1- |
| 7460 | ethanediyl]bis[N-(carboxymethyl)glycinato-κN,κO]]] |
| | ruthenium (III) |
| AMD | Dihydrogen [[N,N′-[(butylimino-κN)di-2,1-ethanediyl]bis[N- |
| 8676 | (carboxymethyl)glycinato-κN,κO]]]chloro ruthenium (III) |
| AMD | Dihydrogen chloro[[N,N′-[(ethylimino-κN)di-2,1-ethanediyl] |
| 8679 | bis[N-(carboxymethyl)glycinato-κN,κO]]] ruthenium (III) |
| AMD | Dihydrogen chloro[[N,N′-[(phenylimino-κN)di-2,1- |
| 8684 | ethanediyl]bis[N-(carboxymethyl)glycinato-κN,κO]]] |
| | ruthenium (III) |
| AMD | [N-[2-[[(carboxy-κO)methyl][(2-pyridinyl-κN)methyl]amino-κN] |
| 7436 | ethyl-N-[2-[(carboxymethyl)[(2-pyridinyl-κN]methyl]amino-κN] |
| | ethyl]glycinato-κN] ruthenium (III) bis(trifluoroacetate) |
| AMD | Potassium dihydrogen dichloro[[N,N′-1,3-propanediylbis[N- |
| 8701 | (carboxymethyl)glycinato-κN,κO]]] ruthenium (III) |
| AMD | Hydrogen aqua[6-[[[(carboxy-κO)methyl](carboxymethyl) |
| 7494 | amino-κN]methyl]-2-pyridinecarboxylato-κN¹,κO²]chloro |
| | ruthenium (III) |
| AMD | Hydrogen aqua[N-(carboxymethyl)-N-[[6-(hydroxymethyl)-2- |
| 7493 | pyridinyl-κN]methyl]glycinato-κN,κO]dichloro ruthenium (III) |
| AMD | Aqua[N-[(carboxy-κO)methyl]-N-[[6-[(phenylmethoxy)methyl]- |
| 8699 | 2-pyridinyl-κN]methyl]glycinato-κN,κO]chloro ruthenium (III) |
| AMD | Potassium chloro[methyl 3-[[[2-[bis[(carboxy-κO)methyl]amino- |
| 8677 | κN]ethyl][(carboxy-κO)methyl]amino-κN]methyl]benzoato |
| | ruthenium (III) |
| AMD | Aqua[N-[2-[bis[(carboxy-κO)methyl]amino-κN]ethyl]- |
| 8893 | N-[2-oxo-2-(1-pyrrolidinyl)ethyl]glycinato-κN,κO] |
| | ruthenium (III) |
| AMD | Potassium aqua[N-[2-[bis[(carboxy-κO)methyl]amino- |
| 8894 | κN]ethyl]-N-[(carboxy-κO)methyl]glycyl-κN-L-isoleucinato |
| | ruthenium (III) |
| AMD | Hydrogen aqua[N-[2-[[(carboxy-κO)methyl](carboxymethyl) |
| 8711 | amino-κN]ethyl]-N-(phenylmethyl)glycinato-κN,κO]chloro |
| | ruthenium (III) |
| AMD | Dihydrogen aqua[3-[[[(carboxy-κO)methyl][2-[[(carboxy- |
| 8702 | κO)methyl](carboxymethyl)amino-κN]ethyl]amino-κN]methyl] |
| | benzoato]chloro ruthenium (III) |
| AMD | Aquachloro[[N,N′-1,2-ethanediylbis[N-[2-oxo-2-(1- |
| 8849 | pyrrolidinyl)ethyl]glycinato-κN,κO]]] ruthenium (III) |
| AMD | Dihydrogen aqua[[N,N′-(2-hydroxy-1,3-propanediyl)bis[N- |
| 7461 | (carboxymethyl)glycinato-κN,O]]](trifluoromethanesulfonato-κO) |
| | ruthenium (III) |
| AMD | Potassium dichloro[[N,N′-1,2-ethanediylbis[glycinato-κN,κO]] |
| 7462 | ruthenium (III) |
| AMD | Chloro[octahydro-1H-1,4,7-triazoninato-κN¹,κN⁴, |
| 8672 | κN⁷]bis[(sulfinyl-κS)bis[methane] ruthenium (II) chloride |
| AMD | Trichloro[octahydro-1H-1,4,7-triazonine-κN¹,κN⁴,κN⁷] |
| 8641 | ruthenium (III) |
| AMD | Trichloro[hexahydro-1,4,7-trimethyl-1,4,7-triazonine-κN¹,κN⁴, |
| 8671 | κN⁷] ruthenium (III) |
| AMD | (Dimethylcarbamodithioato-κS)(dimethylcarbamodithioato-κS,κS′) |
| 8670 | [octahydro-1H-1,4,7-triazonine-κN¹,κN⁴,κN⁷] ruthenium (III) |
| | hexafluorophosphate |
| AMD | (Diethylcarbamodithioato-κS)(diethylcarbamodithioato-κS,κS′) |
| 8803 | [octahydro-1H-1,4,7-triazonine-κN¹,κN⁴,κN⁷] |
| | ruthenium (III) hexafluorophosphate |
| AMD | (1,4-butanediylcarbamodithioato-κS)(1,4-butanediylcarbamodi- |
| 8842 | thioato-κS,κS′) [octahydro-1H-1,4,7-triazonine-κN¹,κN⁴,κN⁷] |
| | ruthenium (III) hexafluorophosphate |
| AMD | Dihydrogen ((1-carboxy)-1,4-butanediylcarbamodithioato-κS) |
| 8731 | ((1-carboxy)-1,4-butanediylcarbamodithioato-κS,κS′) [octahydro- |
| | 1H-1,4,7-triazonine-κN¹,κN⁴,κN⁷] ruthenium (III) |
| | hexafluorophosphate |
| AMD | ((1-carboxymethyl)-1,4-butanediylcarbamodithioato-κS) |
| 8802 | ((1-carboxymethyl)-1,4-butanediylcarbamodithioato-κS,κS′) |
| | [octahydro-1H-1,4,7-triazonine-κN¹,κN⁴,κN⁷] |
| | ruthenium (III) hexafluorophosphate |
| AMD | Dihydrogen (N-methyl-N-sec-butylcarboxycarbamodithioato-κS) |
| 8801 | (N-methyl-N-sec-butylcarboxycarbamodithioato-κS,κS′) |
| | [octahydro-1H-1,4,7-triazonine-κN¹,κN⁴,κN⁷] |
| | ruthenium (III) hexafluorophosphate |
| AMD | (Dimethylcarbamodithioato-κS)(dimethylcarbamodithioato-κS,κS′) |
| 8682 | [hexahydro-1,4,7-trimethyl-1,4,7-triazonine-κN¹,κN⁴,κN⁷] |
| | ruthenium (III) hexafluorophosphate |
| AMD | [(N-(carboxy-κO)-methyl)-N-methylglycinato-κN,κO][octahydro- |
| 8800 | 1H-1,4,7-triazonine-κN¹,κN⁴,κN⁷] ruthenium (III) |
| | hexafluorophosphate |
| AMD | Hydrogen chloro[hexahydro-1,4,7-(tricarboxy-κO,κO′-methyl)- |
| 8811 | 1,4,7-triazonine-κN¹,κN⁴,κN⁷] ruthenium (III) |
| AMD | Chloro(2,2′-bipyridine-κN¹,κN¹′)(2,2′:6′.2″-terpyridine- |
| 7044 | κN¹,κN²′,κN¹″) ruthenium (II) hexafluorophosphate |
| AMD | Chlorobis(2(1H)-pyridinethione-κS²)(2,2′:6′.2″-terpyridine-κN¹, |
| 7054 | κN²,κN¹″) ruthenium (II) hexafluorophosphate |
| AMD | Chlorobis(2(1H)-pyrimidinethione-κS²)(2,2′:6′.2″-terpyridine-κN¹, |
| 7055 | κN²′,κN¹″) ruthenium (II) hexafluorophosphate |
| AMD | Chloro(dimethylcarbamodithioato-κS,κS′)(2,2′:6′.2″-terpyridine- |
| 7086 | κN¹,κN²′,κN¹″) ruthenium (III) hexafluorophosphate |
| AMD | Dichlorobis(2,2′-bipyridine-κN¹,κN¹″) |
| 7036 | ruthenium (II) dihydrate |
| AMD | Dichlorobis(1,10-phenanthroline-κN¹,κN¹⁰) |
| 7037 | ruthenium (II) dihydrate |
| AMD | Bis(2,2′-bipyridine-κN¹,κN¹′)(2(1H)-pyridinethionato-κN¹, |
| 7039 | κS²) ruthenium (II) perchloate |
| AMD | Bis(2,2′-bipyridine-κN¹,κN¹′)(2(1H)-pyridinethionato-κN¹, |
| 7045 | κS²) ruthenium (II) hexafluorophosphate |
| AMD | Bis(acetonitrile)bis(2,4-pentanedionato-κO,κO′) ruthenium (III) |
| 8657 | trifluoromethanesulfonate |
| AMD | Bis(acetonitrile)bis(2,4-pentanedionato-κO,κO′) ruthenium (II) |
| 8660 | |
| AMD | Bis(acetonitrile)bis(3-methyl-2,4-pentanedionato-κO,κO′) |
| 8892 | ruthenium (III) trifluoromethanesulfonate |
| AMD | Bis(acetonitrile)bis(3-methyl-2,4-pentanedionato-κO,κO′) |
| 8901 | ruthenium (II) |
| AMD | Bis(acetonitrile)bis(3-chloro-2,4-pentanedionato-κO,κO′) |
| 8883 | ruthenium (II) |
| AMD | Bis(acetonitrile)bis(3-chloro-2,4-pentanedionato-κO,κO′) |
| 8884 | ruthenium (III) trifluoromethanesulfonate |
| AMD | Bis(acetonitrile)bis(3-bromo-2,4-pentanedionato-κO,κO′) |
| 8881 | ruthenium (III) trifluoromethanesulfonate |
| AMD | Bis(acetonitrile)bis(3-bromo-2,4-pentanedionato-κO,κO′) |
| 8900 | ruthenium (II) |
| AMD | Bis(acetonitrile)(2,4-pentanedionato-κO,κO′)(3-iodo-2,4- |
| 8910 | pentanedionato-κO,κO′) ruthenium (III) |
| | trifluoromethanesulfonate |
| AMD | Tetrakis(acetonitrile)(3-iodo-2,4-pentanedionato-κO,κO′) |
| 8896 | ruthenium (II) trifluoromethanesulfonate |
| AMD | Bis(acetonitrile)bis(1,3-diphenyl-1,3-propanedionato-κO,κO′) |
| 8691 | ruthenium (III) trifluoromethanesulfonate |
| AMD | Bis(acetonitrile)bis(1,3-diphenyl-1,3-propanedionato-κO,κO′) |
| 8692 | ruthenium (II) |
| AMD | Bis(acetonitrile)bis(2,2,6,6-tetramethyl-3,5-heptanedionato- |
| 8707 | κO,κO′) ruthenium (III) trifluoromethanesulfonate |
| AMD | Bis(acetonitrile)bis(1,1,1,5,5,5-hexafluoro-2,4-pentanedionato- |
| 8658 | κO,κO′) ruthenium (II) |
| AMD | sym-Bis(acetonitrile)bis(1,1,1-trifluoro-2,4-pentanedionato- |
| 8693 | κO,κO′) ruthenium (II) |
| AMD | asym-Bis(acetonitrile)bis(1,1,1-trifluoro-2,4-pentanedionato- |
| 8694 | κO,κO′) ruthenium (II) |
| AMD | sym-Bis(acetonitrile)bis(1,1,1-trifluoro-5,5-dimethyl-2,4- |
| 8730 | hexanedionato-κO,κO′) ruthenium (II) |
| AMD | asym-Bis(acetonitrile)bis(1,1,1-trifluoro-5,5-dimethyl-2,4- |
| 8710 | hexanedionato-κO,κO′) ruthenium (II) |
| AMD | Bis(acetonitrile)bis](3-hydroxy-κO)-2-methyl-4-pyronato-κO′] |
| 8757 | ruthenium (III) trifluoromethanesulfonate |
| AMD | Bis(acetonitrile)bis[4-(hydroxy-κO)-3-penten-2-onato](N,N,N′,N′- |
| 8695 | tetramethyl-1,3-propanediamine-κN,κN′) ruthenium (III) |
| | trifluoromethanesulfonate |
| AMD | Bis(acetonitrile)bis[4-(hydroxy-κO)-3-penten-2-onato] |
| 8696 | bis(N,N,N′,N′-tetramethyl-1,3-propanediamine-κN) ruthenium |
| | (III) trifluoromethanesulfonate |
| AMD | Bis(acetonitrile)[N,N′-bis[2-(amino-κN)ethyl]amine]bis[4- |
| 8704 | (hydroxy-κO)-3-penten-2-onato] ruthenium (III) |
| | trifluoromethanesulfonate |
| AMD | Bis(acetonitrile)[N-(2-aminoethyl)-1,2-ethanediamine-κN,κN′]bis |
| 8705 | [4-(hydroxy-κO)-3-penten-2-onato] ruthenium (III) |
| | trifluoromethanesulfonate |
| AMD | Bis(acetonitrile)[2-(2-amino-κN-ethylamino-κN′)ethanol]bis[4- |
| 8874 | (hydroxy-κO)-3-penten-2-onato] ruthenium (III) |
| | trifluoromethanesulfonate |
| AMD | Bis(acetonitrile)[N-(3-aminopropyl)-1,3-propanediamine-κN, |
| 8878 | κN′]bis[4-(hydroxy-κO)-3-penten-2-onato] |
| | ruthenium (III) trifluoromethanesulfonate |
| AMD | Bis(acetonitrile)[N-(2-aminoethyl)-1,3-propanediamine-κN, |
| 8879 | κN′]bis[4-(hydroxy-κO)-3-penten-2-onato] |
| | ruthenium (III) trifluoromethanesulfonate |
| AMD | Bis(acetonitrile)[N,N-bis[2-(amino-κN)ethyl]-L-isoleucyl- |
| 8813 | L-prolinato]bis[4-(hydroxy-κO)-3-penten-2-onato] ruthenium (III) |
| | trifluoromethanesulfonate |
| AMD | (Dimethylcarbamodithioato-κS,κS′)bis(2,4-pentanedionato- |
| 8656 | κO,κO′) ruthenium (III) |
| AMD | (Dimethylcarbamodithioato-κS,κS′)bis(1,3-diphenyl- |
| 8792 | 1,3-propanedionato-κO,κO′) ruthenium (III) |
| AMD | [(1-carboxymethyl)-1,4-butanediylcarbamodithioato-κS, |
| 8822 | κS′]bis(2,4-pentanedionato-κO,κO′) ruthenium (III) |
| AMD | [(1-carboxymethyl)-1,4-butanediylcarbamodithioato- |
| 8823 | κS,κS′]bis(1,3-diphenyl-1,3-propanedionato- |
| | κO,κO′) ruthenium (III) |
| AMD | [L-prolinato(1-)-κN,κO]bis(1,3-diphenyl-1,3-propanedionato- |
| 8826 | κO,κO′) ruthenium (III) |
| AMD | Potassium [(1-carboxy)-1,4-butanediylcarbamodithioato-κS, |
| 8736 | κS′]bis(2,4-pentanedionato-κO,κO′) ruthenium (III) |
| AMD | [N-methyl-L-isoleucinato(1-)-κN,κO]bis(2,4-pentanedionato- |
| 8791 | κO,κO′) ruthenium (III) |
| AMD | Bis[μ-[N-methyl-L-isoleucinato(1-)-κN:κO]]tetrakis |
| 8795 | (2,4-pentanedionato-κO,κO′) diruthenium (III) |
| AMD | Bis[μ-[L-prolinato(1-)-κN:κO]]tetrakis(1,3-diphenyl-1,3- |
| 8845 | propanedionato-κO,κO′) diruthenium (III) |
| AMD | bis(2,4-pentanedionato-κO,κO′)[2(1H)-pyridinethionato- |
| 8856 | κS²][2(1H)-pyridinethione-κS²] ruthenium (III) |
| AMD | bis(2,4-pentanedionato-κO,κO′)[2(1H)-pyridinethionato-κN, |
| 8857 | κS²] ruthenium (III) |
| AMD | bis(2,4-pentanedionato-κO,κO′)bis[4-(1H-imidazol-1-yl- |
| 8865 | κN³)phenol] ruthenium (III) trifluoromethanesulfonate |
| AMD | (Acetonitrile)bis(1,3-diphenyl-1,3-propanedionato-κO, |
| 8873 | κO′)[4-(1H-imidazol-1-yl-κN³)phenol] |
| | ruthenium (III) trifluoromethanesulfonate |
| AMD | bis(1,3-diphenyl-1,3-propanedionato-κO,κO′) |
| 8877 | bis[4-(1H-imidazol-1-yl-κN³)phenol] ruthenium |
| | (III) trifluoromethanesulfonate |
| AMD | Bis[methyl-1-[(1H-imidazol-1-yl-κN³)acetyl]-L-prolinate]bis(2,4- |
| 8866 | pentanedionato-κO,κO′) ruthenium (III) trifluoromethanesulfonate |
| AMD | (Acetonitrile)(4-ethylamino-1H-imidazol-κN³)bis(2,4- |
| 8891 | pentanedionato-κO,κO′) ruthenium (III) |

1. A compound of the formula
[Mₐ(X_{b}L)_{c}Y_{d}Zₑ]^{nt÷} Formula I
wherein:
M is a metal ion or a mixture of metal ions;
X is a cation or a mixture of cations;
L is a ligand, or mixture of ligands each containing at least two different donor atoms selected from the elements of Group IV, Group V or Group VI of the Periodic Table;
Y is a ligand or a mixture of the same or different ligands each containing at least one donor atom or more than one donor atom selected from the elements of Group IV, Group V or Group VI of the Periodic Table; and
Z is a halide or pseudohalide ion or a mixture of halide ions and pseudohalide ions; and
wherein: a=1-3; b=0-12; c=0-18; d=0-18; e=0-18; and n=0-10; provided that at least one of c, d and e is 1 or more;
wherein c is 0: b is also 0;
wherein a is 1: c, d and e are not greater than 9; and
wherein a is 2: c, d and e are not greater than 12;
including any pharmaceutically acceptable salts thereof and any stereoisomeric forms and mixtures of stereoisomeric forms thereof.

2. A neutral, anionic or cationic metal complex having at least one site for coordination with NO of Formula I:
[Mₐ(XbL)_{c}Y_{d}Zₑ]^{nt÷} Formula I
useful in the manufacture of a medicament for the attenuation of NO levels and other reactive oxygen species when implicated in disease, where:
M is a metal ion or a mixture of metal ions:
X is a cation or a mixture of cations:
L is a ligand, or mixture of ligands each containing at least two different donor atoms selected from the elements of Group IV, Group V or Group VI of the Periodic Table;
Y is a ligand or a mixture of the same or different ligands each containing at least one donor atom or more than one donor atom which donor atom is selected from the elements of Group IV, Group V or Group VI of the Periodic Table:
And
Z is a halide or pseudohalide ion or a mixture of halide ions and pseudohalide ions:
a=1-3; b=0-12; c=0-18; d=0-18; e=0-18; and n=0-10; provided that at least one of c, d and e is 1 or more.
And where c is 0: b is also 0;
And where a is 1: c, d and e are not greater than 9;
And where a is 2: c, d and e are not greater than 12.

3. The compound or composition f any one of claims 1, 6-10 or the complex of claim 2, wherein M is a first, second or third row transition metal ion or is in oxidation state III or is selected from the group consisting of: Rh, Ru, Os, Mn, Co, Cr and Re.

4. The compound of claim 1 or the complex of claim 2, wherein X is a mono-, di- or tri-valent cation or is selected from the group consisting of: H⁺, K⁺, Na⁺, NH₄⁺ and Ca^{2+.}

5. The compound of claim 1 or the complex of claim 2, wherein L is selected from the group consisting of: tropolone; ethylenediamine-N,N′-diacetic acid (edda), ethylenediaminetetraacetic acid (edta), nitrilotriacetic acid (nta), dipicolinic acid (dipic), picolinic acid (pic), diethylenetri-aminepentaacetic acid (dtpa), thiobis(ethylenenitrilo)tetraacetic acid (tedta), dithioethanebis(ethylenenitrilo)tetraacetic acid (dtedta), N-(2-hydroxethyl) ethylenediamine-triacetic acid (hedtra), diamide of edta, diamide of dtpa, an amide or ester derivative thereof or a mixture of any one of these or L^{II} is a polydentate aminocarboxylate ligand.

6. A composition comprising an optionally hydrated ruthenium complex of Formula II:
[Ru(H₀₋₆ L^{II})₁₋₃Y₀₋₂Cl₀₋₄]^{(0-4)÷} Formula II
where L^{II} a ligand or a mixture of the same or different ligands each selected from the group consisting of: tropolone; ethylenediamine-N,N′-diacetic acid (edda), ethylenediaminetetraacetic acid (edta), nitrilotriacetic acid (nta), dipicolinic acid (dipic), picolinic acid (pic), diethylenetri-aminepentaacetic acid (dtpa), thiobis(ethylenenitrilo)tetraacetic acid (tedta), dithioethanebis(ethylenenitrilo)tetraacetic acid (dtedta), N-(2-hydroxethyl) ethylenediamine-triacetic acid (hedtra), diamide of edta, diamide of dtpa, an amide or ester derivative thereof or a mixture of any one of these or L^{II} is a polydentate aminocarboxylate ligand;
Y is a ligand or a mixture of the same or different ligands each containing at least one donor atom or more than one donor atom which donor atom is selected from the elements of Group IV, Group V or Group VI of the Periodic Table;
including any pharmaceutically acceptable salts thereof and any stereoisomeric forms and mixtures of stereoisomeric forms thereof.

7. A composition comprising an optionally hydrated ruthenium complex of Formula II:
[Ru(H₀₋₆L^{II})₁₋₃Y₀₋₂Cl₀₋₄]^{(0-4)÷} Formula II
where L^{II} a ligand or a mixture of the same or different ligands each selected from the group consisting of: tropolone; ethylenediamine-N,N′-diacetic acid (edda), ethylenediaminetetraacetic acid (edta), nitrilotriacetic acid (nta), dipicolinic acid (dipic), picolinic acid (pic), diethylenetri-aminepentaacetic acid (dtpa), thiobis(ethylenenitrilo)tetraacetic acid (tedta), dithioethanebis(ethylenenitrilo)tetraacetic acid (dtedta), N-(2-hydroxethyl) ethylenediamine-triacetic acid (hedtra), diamide of edta, diamide of dtpa, an amide or ester derivative thereof or a mixture of any one of these or L^{II} is a polydentate aminocarboxylate ligand;
Y is a ligand or a mixture of the same or different ligands each containing at least one donor atom or more than one donor atom which donor atom is selected from the group consisting of: acetylacetone (acac) a β-diketonate; water; dimethylsulphoxide (dmso); carboxylate; bidentate carboxylate; catechol; kojiic acid; maltol; hydroxide; tropolone; malonic acid; oxalic acid; 2.3-dihydroxynaphthalene; squaric acid; acetate; a sulphate and a glycolate; and
including any pharmaceutically acceptable salts thereof and any stereoisomeric forms and mixtures of stereoisomeric forms thereof.

8. The composition of claim 6 or 7, selected from the group consisting of:
K[Ru(Hedta)Cl]2H₂O; [Ru(H₂edta)(acac)]; K[Ru(hedtra)Cl]H₂O; K[Ru(dipic)₂]H₂O; (H₂pic)[RuCl₂Pic)₂](Hpic)H₂O; K[Ru(H₂edta)Cl₂]H₂O; K[Ru(Hnta)₂]½H₂O; K[Ru(H₂dtpa)Cl]H₂O; [Ru(Hhedtra)acac]H₂O; [Ru(Hhedtra)trop]; and [Ru(H₃dtpa)Cl].

9. An optionally hydrated complex of the formula:
[M₁₋₃Y₁₋₁₈Cl₀₋₁₈]^{(0-6)÷} Formula III
wherein:
M is a metal ion or a mixture of metal ions;
Y is a ligand or a mixture of the same or different ligands each containing at least one donor atom or more than one donor atom selected from the elements of Group IV, Group V or Group VI of the Periodic Table.

10. The complex of claim 9, wherein Y is a sulphur donor ligand.

11. The complex of claim 9 or 10, wherein said complex is [Ru(mtc)₃] or Ru(S₂CNCH₂CH₂NMeCH₂CH₂)₃½H₂O, wherein mtc is 4-morpolinecarbodithoic acid.

12. An optionally hydrated complex of the formula:
[M^{III}₁₋₃Y^{III}₁₋₁₈Cl₀₋₁₈]^{(0-6)÷} Formula III
where M^{III} is ruthenium and y^{III} is an oxygen-donor ligand, selected from the group consisting of: acetate, lactate, water, oxide, propionate (COEt), oxalate (ox), and maltolate (maltol), and a combination thereof.

13. The complex of any one of claims 9-12, wherein said complex is selected from the group consisting of: [Ru₃O(OAc)₆](OAc); [Ru₃O(lac)₆](lac); [Ru₂(OAc)₄]NO₃; [Ru₂(OCOEt)₄]NO₃; K₃[Ru(ox)₃];[Ru₂(OAc)₄]Cl; and [Ru(maltol)₃].

14. An optionally hydrated complex of the formula:
[RuY^{IV}₁₋₉Cl₁₋₉]^{(0-4)÷} Formula IV
where Y^{IV} is a nitrogen-donor ligand.

15. The complex of claim 14, wherein Y^{IV} is selected from the group consisting of: ammine; ethylenediamine (en); pyridine (py); 1,10-phenanthroline (phen): 2,2-bipyridine (bipy) or 1,4,8,11-tetraazacyclotetradecane (cyclam); 1,4,7-triazacyclononane; 1,4,7-triazacyclononane tris acetic acid; 2,3,7,8,12,13,17,18-octaethylporphyrin (oep); and a combination thereof.

16. The complex of claim 14 or claim 15, wherein said complex is selected from the group consisting of: [Ru(H₃N)₅Cl]Cl₂; [Ru(en)₃]II₃; trans-[RuCl₂(py)₄]; K[Ru(Phen)Cl₄]; [Ru(cyclam)Cl₂]Cl; K[Ru(bipy)Cl₄]; [Ru(NH₃)₆]Cl₃; [Ru(NH₃)₄Cl₂]Cl; Ru(oep)Ph; and any combination thereof.

17. An optionally hydrated complex of the formula:
[M₁₋₃Y^{V}₁₋₁₈Cl₀₋₁₈]^{(0-6)÷} Formula V
where Y^{V} is a combination of donor ligands.

18. The complex of claim 17, wherein Y^{V} is selected from the group consisting of: ammine; dmso; oxalate; bipy; acac; methyl cyanide; and any combination thereof.

19. The complex of claim 17 or claim 18, wherein said complex is selected from the group consisting of: [Ru(NH₃)(dmso)₂Cl₃]; cis-[Ru(dmso)₄Cl₂]; cis-[Ru(NH₃)(dmso)₃Cl₂]; [Ru(dmso)₃Cl₃]; [Os(ox)(bipy)₂]H₂O; [Ru(acac)₂(MeCN)₂]CF₃SO₃; and combinations thereof.

20. A pharmaceutical composition comprising an optionally hydrated complex of formula [Os(ox)(bipy)₂].

21. A pharmaceutical composition comprising an optionally hydrated complex of formula [Ru(acac)₂(MeCN)₂]⁺.

22. An optionally hydrated complex selected from the group consisting of:
(a) AMD 7040, Dihydrogen chloro[[2,6-(pyridinyl-κN)methyl]bis[N-(carboxymethyl)glycinato-κN,κO]] ruthenium (III);
(b) AMD 7043, Dihydrogen dichloro[[N,N′-1,2-ethanediyl]bis[(2-pyridinyl-κN)methylglycinato-κN] ruthenium (III) chloride;
(c) AMD 7056, Aquachloro[[N-2-[(2-pyridinyl-κN)oxo-methyl)aminoethyl][((2-carboxy-κO)methyl)glycinato-κN,κO]] ruthenium (III);
(d) AMD 7046, Hydrogen chloro[N-[bis((2-(carboxy-κO)methyl)imino-κN)ethyl]-(2-pyridinyl-κN)methylglycinato-κN] ruthenium (III);
(e) AMD 7087, Hydrogen aqua[N-bis((2-carboxy-κO)methyl)imino-κN]-1,2-phendiyl(2-(carboxy-κO)methyl)glycinato-κN] ruthenium (III);
(f) AMD 7459, Dihydrogen chloro[[N,N′-[[(phenylmethyl)imino-κN]-2,1-ethanediyl]bis[N-(carboxymethyl)glycinato-κN,κO]] ruthenium (III);
(g) AMD 7460, Dihydrogen chloro[[N,N′-[[(2-pyridinylmethyl)imino-κN]di-2,1-ethanediyl]bis[N-(carboxymethyl)glycinato-κN,κO]]] ruthenium (III);
(h) AMD 8676, Dihydrogen [[N,N′-[(butylimino-κN)di-2,1-ethanediyl]bis[N-(carboxymethyl)glycinato-κN,κO]]]chloro ruthenium (III);
(i) AMD 8679, Dihydrogen chloro[[N,N′-[(ethylimino-κN)di-2,1-ethanediyl]bis[N-(carboxymethyl)glycinato-κN,κO]]] ruthenium (III);
(j) AMD 8684, Dihydrogen chloro[[N,N′-[(phenylimino-κ)di-2,1-ethanediyl]bis[N-(carboxymethyl)glycinato-κN,κO]]] ruthenium (III);
(k) AMD 7436, [N-[2-[[(carboxy-κO)methyl][(2-pyridinyl-κN)methyl]amino-κN]ethyl-N-[2-[(carboxymethyl)[(2-pyridinyl-κN]methyl]amino-κN]ethyl]glycinato-κN] ruthenium (III) bis(trifluoroacetate);
(l) AMD 8701, Potassium dihydrogen dichloro[[N,N′-1,3-propanediylbis[N-(carboxymethyl)glycinato-κN,κO]]] ruthenium (III);
(m) AMD 7494, Hydrogen aqua[6-[[[(carboxy-κO)methyl](carboxymethyl)amino-κN]methyl]-2-pyridinecarboxylato-κN¹, κO²]chloro ruthenium (III);
(n) AMD 7493, Hydrogen aqua[N-(carboxymethyl)-N-[[6-(hydroxymethyl)-2-pyridinyl-κN]methyl]glycinato-κN,κO] dichloro ruthenium (III);
(o) AMD 8699, Aqua[N-[(carboxy-κO)methyl]-N-[[6-[(phenylmethoxy)methyl]-2-pyridinyl-κN]methyl]glycinato-κN,κO]chloro ruthenium (III);
(p) AMD 8677, Potassium chloro[methyl 3-[[[2-[bis[(carboxy-κO)methyl]amino-κN]ethyl][(carboxy-κO)methyl]amino-κN]methyl]benzoato ruthenium (III);
(q) AMD 8893, Aqua[N-[2-[bis[(carboxy-κO)methyl]amino-κN]ethyl]-N-[2-oxo-2-(1-pyrrolidinyl)ethyl]glycinato-κN,κO] ruthenium (III);
(r) AMD 8894, Potassium aqua[N-[2-[bis[(carboxy-κO)methyl]amino-κN]ethyl]-N-[(carboxy-κO)methyl]glycyl-κN-L-isoleucinato ruthenium (III);
(s) AMD 8711, Hydrogen aqua[N-[2-[[(carboxy-κO)methyl](carboxymethyl)amino-κN]ethyl]-N-(phenylmethyl)glycinato-κN,κO]chloro ruthenium (III)
(t) AMD 8702, Dihydrogen aqua[3-[[[(carboxy-κO)methyl][2-[[(carboxy-κO)methyl](carboxymethyl)amino-κN]ethyl]amino-κN]methyl]benzoato]chloro ruthenium (III)
(u) AMD 8849, Aquachloro[[N,N′-1,2-ethanediylbis[N-[2-oxo-2-(1-pyrrolidinyl)ethyl]glycinato-κN,κO]]] ruthenium (III);
(v) AMD 7461, Dihydrogen aqua[[N,N′-(2-hydroxy-1,3-propanediyl)bis[N-(carboxymethyl)glycinato-κN,κO]]](trifluoromethanesulfonato-κO) ruthenium (III)
(w) AMD 7462, Potassium dichloro[[N,N′-1,2-ethanediylbis[glycinato-κN,κO]] ruthenium (III)
(x) AMD 8672, Chloro[octahydro-1H-1,4,7-triazoninato-κN¹,κN⁴,κN⁷]bis[(sulfinyl-κS)bis[methane] ruthenium (II) chloride
(y) AMD 8641, Trichloro[octahydro-1H-1,4,7-triazonine-κN¹,κN⁴,κN⁷] ruthenium (III)
(z) AMD 8671, Trichloro[hexahydro-1,4,7-trimethyl-1,4,7-triazonine-κN¹,κN⁴,κN⁷] ruthenium (III)
(aa) AMD 8670, (Dimethylcarbamodithioato-κS)(dimethylcarbamodithioato-κS,κS′) [octahydro-1H-1,4,7-triazonine-κN¹,κN⁴,κN⁷] ruthenium (III) hexafluorophosphate
(bb) AMD 8803, (Diethylcarbamodithioato-κS)(diethylcarbamodithioato-κS,κS′) [octahydro-1H-1,4,7-triazonine-κN¹,κN⁴,κN⁷] ruthenium (III) hexafluorophosphate;
(cc) AMD 8842, (1,4-butanediylcarbamodithioato-κS)(1,4-butanediylcarbamodithioato-κS,κS′) [octahydro-1H-1,4,7-triazonine-κN¹,κN⁴,κN⁷] ruthenium (III) hexafluorophosphate;
(dd) AMD 8731, Dihydrogen ((1-carboxy)-1,4-butanediylcarbamodithioato-κS)((1-carboxy)-1,4-butanediylcarbamodithioato-κS,κS′) [octahydro-1H-1,4,7-triazonine-κN¹,κN⁴,κN⁷] ruthenium (III) hexafluorophosphate;
(ee) AMD 8802, ((1-carboxymethyl)-1,4-butanediylcarbamodithioato-κS)((1-carboxymethyl)-1,4-butanediylcarbamodithioato-κS,κS′) [octahydro-1H-1,4,7-triazonine-κN¹,κN⁴,κN⁷] ruthenium (III) hexafluorophosphate;
(ff) AMD 8801, Dihydrogen (N-methyl-N-sec-butylcarboxycarbamodithioato-κS)(N-methyl-N-sec-butylcarboxycarbamodithioato-κS,κS′) [octahydro-1H-1,4,7-triazonine-κN¹,κN⁴,κN⁷] ruthenium (III) hexafluorophosphate;
(gg) AMD 8682, (Dimethylcarbamodithioato-κS)(dimethylcarbamodithioato-κS,κS′) [hexahydro-1,4,7-trimethyl-1,4,7-triazonine-κN¹,κN⁴,κN⁷] ruthenium (III) hexafluorophosphate;
(hh) AMD 8800, [(N-(carboxy-κO)-methyl)-N-methylglycinato-κN,κO][octahydro-1H-1,4,7-triazonine-κN¹,κN⁴,κN⁷] ruthenium (III) hexafluorophosphate;
(ii) AMD 8811, Hydrogen chloro[hexahydro-1,4,7-(tricarboxy-κO,κO′-methyl)-1,4,7-triazonine-κN¹,κN⁴,κN⁷] ruthenium (III);
(jj) AMD 7044, Chloro(2,2′-bipyridine-κN¹,κN¹′)(2,2′:6′.2″-terpyridine-κN¹,κN²′,κN¹″) ruthenium (II) hexafluorophosphate;
(kk) AMD 7054, Chlorobis(2(1B)-pyridinethione-κS²)(2,2′:6′.2″-terpyridine-κN¹,κN²′,κN¹″) ruthenium (II) hexafluorophosphate;
(ll) AMD 7055, Chlorobis(2(1H)-pyrimidinethione-κS²)(2,2′:6′.2″-terpyridine-κN¹,κN²′,κN¹″) ruthenium (II) hexafluorophosphate;
(mm) AMD 7086, Chloro(dimethylcarbamodithioato-κS,κS′)(2,2′:6′.2″-terpyridine-κN¹,κN²′,κN¹″) ruthenium (III) hexafluorophosphate;
(nn) AMD 7036, Dichlorobis(2,2′-bipyridine-κN¹,κN¹′) ruthenium (II) dihydrate;
(oo) AMD 7037, Dichlorobis(1,10-phenanthroline-κN¹,κN¹⁰) ruthenium (II) dihydrate;
(pp) AMD 7039, Bis(2,2′-bipyridine-κ¹,ƒ¹′)(2(1H)-pyridinethionato-κN¹,κS²) ruthenium (II) perchlorate;
(qq) AMD 7045, Bis(2,2′-bipyridine-κN¹,κN¹′)(2(1H)-pyridinethionato-κN¹,κS²) ruthenium (II) hexafluorophosphate;
(rr) AMD 8657, Bis(acetonitrile)bis(2,4-pentanedionato-κO,κO′) ruthenium (III) trifluoromethanesulfonate;
(ss) AMD 8660, Bis(acetonitrile)bis(2,4-pentanedionato-κO,κO′) ruthenium (II);
(tt) AMD 8892, Bis(acetonitrile)bis(3-methyl-2,4-pentanedionato-κO,κO′) ruthenium (III) trifluoromethanesulfonate;
(uu) AMD 8901, Bis(acetonitrile)bis(3-methyl-2,4-pentanedionato-κO,κO′) ruthenium (II);
(vv) AMD 8883, Bis(acetonitrile)bis(3-chloro-2,4-pentanedionato-κO,κO′) ruthenium (II);
(ww) AMD 8884, Bis(acetonitrile)bis(3-chloro-2,4-pentanedionato-κO,κO′) ruthenium (III) trifluoromethanesulfonate;
(xx) AMD 8881, Bis(acetonitrile)bis(3-bromo-2,4-pentanedionato-κO,κO′) ruthenium (III) trifluoromethanesulfonate;
(yy) AMD 8900, Bis(acetonitrile)bis(3-bromo-2,4-pentanedionato-κO,κO′) ruthenium (II); and
(zz) AMD 8910, Bis(acetonitrile)(2,4-pentanedionato-κO,κO′)(3-iodo-2,4-pentanedionato-κO,κO′) ruthenium (III) trifluoromethanesulfonate.

23. An optionally hydrated complex selected from the group consisting of:
(a) AMD 8896, Tetrakis(acetonitrile)(3-iodo-2,4-pentanedionato-κO,κO′) ruthenium (II) trifluoromethanesulfonate;
(b) AMD 8691, Bis(acetonitrile)bis(1,3-diphenyl-1,3-propanedionato-κO,κO′) ruthenium (III) trifluoromethanesulfonate;
(c) AMD 8692, Bis(acetonitrile)bis(1,3-diphenyl-1,3-propanedionato-κO,κO′) ruthenium (II);
(d) AMD 8707, Bis(acetonitrile)bis(2,2,6,6-tetramethyl-3,5-heptanedionato-κO,κO′) ruthenium (III) trifluoromethanesulfonate;
(e) AMD 8658, Bis(acetonitrile)bis(1,1,1,5,5,5-hexafluoro-2,4-pentanedionato-κO,κO′) ruthenium (II);
(f) AMD 8693, sym-Bis(acetonitrile)bis(1,1,1 -trifluoro-2,4-pentanedionato-κO,κO′) ruthenium (II);
(g) AMD 8694, asym-Bis(acetonitrile)bis(1,1,1-trifluoro-2,4-pentanedionato-κO,κO′) ruthenium (II);
(h) AMD 8730, sym-Bis(acetonitrile)bis(1,1,1-trifluoro-5,5-dimethyl-2,4-hexanedionato-κO,κO′) ruthenium (II);
(i) AMD 8710, asym-Bis(acetonitrile)bis(1,1,1-trifluoro-5,5-dimethyl-2,4-hexanedionato-κO,κO′) ruthenium (II);
(j) AMD 8757, Bis(acetonitrile)bis[(3-hydroxy-κO)-2-methyl-4-pyronato-κO′] rutheniun (III) trifluoromethanesulfonate;
(k) AMD 8695, Bis(acetonitrile)bis[4-(hydroxy-κO)-3-penten-2-onato](N,N,N′,N′-tetramethyl-1,3-propanediamine-κN,κN′) ruthenium (III) trifluoromethanesulfonate;
(l) AMD 8696, Bis(acetonitrile)bis[4-(hydroxy-κO)-3-penten-2-onato]bis(N,N,N′,N′-tetramethyl-1,3-propanediamine-κN) ruthenium (III) trifluoromethanesulfonate;
(m) AMD 8704, Bis(acetonitrile)[N,N′-bis[2-(amino-κN)ethyl]amine]bis[4-(hydroxy-κO)-3-penten-2-onato] ruthenium (III) trifluoromethanesulfonate;
(n) AMD 8705, Bis(acetonitrile)[N-(2-aminoethyl)-1,2-ethanediamine-κN,κN′]bis[4-(hydroxy-κO)-3-penten-2-onato] ruthenium (III) trifluoromethanesulfonate;
(o) AMD 8874, Bis(acetonitrile)[2-(2-amino-κN-ethylamino-κN′)ethanol]bis[4-(hydroxy-κO)-3-penten-2-onato] ruthenium (III) trifluoromethanesulfonate;
(p) AMD 8878, Bis(acetonitrile)[N-(3-aminopropyl)-1,3-propanediamine-κN,κN′]bis[4-(hydroxy-κO)-3-penten-2-onato] ruthenium (III) trifluoromethanesulfonate;
(q) AMD 8879, Bis(acetonitrile)[N-(2-aminoethyl)-1,3-propanediamine-κN,κN′]bis[4-(hydroxy-κO)-3-penten-2-onato] ruthenium (III) trifluoromethanesulfonate;
(r) AMD 8813, Bis(acetonitrile)[N,N-bis[2-(amino-κN)ethyl]-L-isoleucyl-L-prolinato]bis[4-(hydroxy-κO)-3-penten-2-onato] ruthenium (III) trifluoromethanesulfonate;
(s) AMD 8656, (Dimethylcarbamodithioato-κS,κS′)bis(2,4-pentanedionato-κO,κO′) ruthenium (III);
(t) AMD 8792, (Dimethylcarbamodithioato-κS,κS′)bis(1,3-diphenyl-1,3-propanedionato-κO,κO′) ruthenium (III);
(u) AMD 8822, [(1-carboxymethyl)-1,4-butanediylcarbamodithioato-κS,κS′]bis(2,4-pentanedionato-κO,κO′) ruthenium (III);
(v) AMD 8823, [(1-carboxymethyl)-1,4-butanediylcarbamodithioato-κS,κS′]bis(1,3-diphenyl-1,3-propanedionato-κO,κO′) ruthenium (III);
(w) AMD 8826, [L-prolinato(1−)-κN,κO]bis(1,3-diphenyl-1,3-propanedionato-κO,κO′) ruthenium (III);
(x) AMD 8736, Potassium[(1-carboxy)-1,4-butanediylcarbamodithioato-κS,κS′]bis(2,4-pentanedionato-κO,κO′) ruthenium (III);
(y) AMD 8791, [N-methyl-L-isoleucinato(1)-κN,κO]bis(2,4-pentanedionato-κO,κO′) ruthenium (III);
(z) AMD 8795, Bis[μ-[N-methyl-L-isoleucinato(1−)-κN:κO]]tetrakis(2,4-pentanedionato-κO,κO′) diruthenium (III);
(aa) AMD 8845, Bis[μ-[L-prolinato(1−)-κN:κO]]tetrakis(1,3-diphenyl-1,3-propanedionato-κO,κO′) diruthenium (III);
(bb) AMD 8856, bis(2,4-pentanedionato-κO,κO′)[2(1H)-pyridinethionato-κS²][2(1H)-pyridinethione-κS²] ruthenium (III);
(cc) AMD 8857, bis(2,4-pentanedionato-κO,κO′)[2(1H)-pyridinethionato-κN,κS²] ruthenium (III);
(dd) AMD 8865, bis(2,4-pentanedionato-κO,κO′)bis[4-(1H-imidazol-1-yl-κN³)phenol] ruthenium (III) trifluoromethanesulfonate;
(ee) AMD 8873, (Acetonitrile)bis(1,3-diphenyl-1,3-propanedionato-κO,κO′)[4-(1H-imidazol-1-yl-κN³)phenol] ruthenium (III) trifluoromethanesulfonate;
(ff) AMD 8877, bis(1,3-diphenyl-1,3-propanedionato-κO,κO′)bis[4-(1H-imidazol-1-yl-κN³)phenol] ruthenium (III) trifluoromethanesulfonate;
(gg) AMD 8866, Bis[methyl-1-[(1H-imidazol-1-yl-κN³)acetyl]-L-prolinate]bis(2,4-pentanedionato-κO,κO′) ruthenium (III) trifluoromethanesulfonate; and
(hh) AMD 8891, (Acetonitrile)(4-ethylamino-1H-imidazol-κN³)bis(2,4-pentanedionato-κO,κO′) ruthenium (III).

24. A method of treating disease in a human or animal subject, wherein said disease results from overproduction of nitric oxide, comprising administering a pharmaceutical composition comprising an optionally hydrated neutral, anionic or cationic metal complex of any of Formulae I-V.

25. A method of attenuation of reactive oxygen species when implicated in diseases of the human body, comprising administering a pharmaceutical composition comprising an optionally hydrated neutral, anionic or cationic metal complex of any of Formulae I-V.

26. A method of attenuation of nitric oxide when implicated in diseases of the human body, comprising administering a pharmaceutical composition comprising an optionally hydrated neutral, anionic or cationic metal complex of any of Formulae I-V.

27. A method of manufacturing a medicament for the treatment of diseases in which reactive oxygen species are overproduced, comprising formulating a pharmaceutical composition comprising an optionally hydrated neutral, anionic, or cationic metal complex of any of Formulae I-V.

28. A pharmaceutical composition comprising a therapeutically effective amount of an active component comprising an optionally hydrated complex of any of Formulae I-V, in admixture with a pharmaceutically acceptable carrier or diluent.

29. The pharmaceutical composition of claim 28, comprising s a dosage range in humans of 1 mg to 10 g per day.

30. The complex according to any one of claims 1-11 or 13, wherein said Ruthenium is complexed to a polyaminocarboxylate ligand of general formulae A and B: 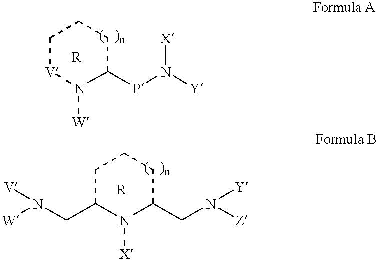
Where:
V′, W′, X′, Y′ and Z′ are independently selected from H, phenyl, heteroaryl, C₁₋₆alkyl, C₁₋₆alkylhydroxy, C₁₋₆alkylthiol, C₁₋₆alkylaryl, C₁₋₆alkylheteroaryl, C₁₋₆alkylheterocyclyl and derivatives thereof;
P′ is: CH₂, (CH₂)₂, CHOHCH₂, or CH(OC₁₋₆alkyl)CH₂; and said ligands may be optionally fused with a heterocyclic ring R (n=0 or 1).

31. The complex of claim 30, wherein said alkylheterocyclic group is selected from the group consisting of: pyridinylmethylene, pyrazinylmethylene, pyrimidinylmethylene.

32. The complex of claim 30, wherein said aromatic and heteroaromatic groups may be suitably substituted in single or multiple positions with halide, C₁₋₆alkyl, C₁₋₆alkoxy, C₁₋₆alkoxyaryl or benzyloxy, hydroxy, C₁₋₆hydroxyalkyl, thiol, carboxylic acid, carboxyalkylC₁₋₆, carboxamide, carboxamidoalkylC₁₋₆, and anilide.

33. The complex of claim 30, wherein said V′, W′, X′, Y′ and Z′ may also be methylenecarboxylic acid, methylenecarboxyC₁₋₆alkyl, methylenecarboxamideC₁₋₆alkyl or heterocyclyl, methylenecarboxanilide, methylenecarboxamido derivatives of an aminoacid or peptide, methylenehydroxamic acid, methylene phosphonic acid, and C₁₋₆alkylthiol.

34. The complex of claim 30, wherein said heterocyclic group is selected from the group consisting of: pyridine, pyrimidine, pyrazine, imidazole, thiazole, and oxazole.

35. A method of inhibiting tumor growth in a mammalian subject, comprising administering to said subject an inhibitory concentration of an optionally hydrated complex of any of Formulae I-V.

36. The method of claim 35, wherein, said complex is AMD6221, K[Ru(H₂dtpa)Cl]H₂O; or AMD6245,[Ru(Hedta)]H₂O.
